(19) United States
(12) Patent Application Publication
(10) Pub. No.: US 2024/0254456 A1
(43) Pub. Date: Aug. 1, 2024

(54) NOVEL PRODUCTION METHOD OF FLAVOCYTOCHROME B2

(71) Applicant: DirectSens GmbH, Klosterneuburg (AT)

(72) Inventors: Peter HERZOG, Obersdorf (AT); Beate STEINER, Korneuburg (AT); Alfons FELICE, Wien (AT); Christopher SCHULZ, Wien (AT)

(73) Assignee: DirectSens GmbH, Klosterneuburg (AT)

(21) Appl. No.: 18/565,765

(22) PCT Filed: Jun. 9, 2022
(86) PCT No.: PCT/EP2022/065632

§ 371 Date: Nov. 30, 2023

(30) Foreign Application Priority Data
Jun. 9, 2021 (EP) 21178543.1

(51) Int. Cl. C12N 9/04; C12Q 1/00

(52) U.S. Cl. CPC C12N 9/0006; C12Q 1/005; C12Y 101/02003

(57) ABSTRACT

A recombinant flavocytochrome b2 (FCb2), comprising a mature FCb2 peptide sequence of a native FCb2, or a functionally active variant of said mature FCb2 peptide sequence, wherein the N-terminus of said mature FCb2 peptide sequence consists of a truncated signal peptide sequence replacing the native signal peptide sequence, and wherein said truncated signal peptide sequence is of the following structure from N- to C-terminus: i. a methionine; ii. optionally a tag sequence; iii. an amino acid sequence of a length of 0-9 amino acids of the native signal peptide sequence, corresponding to the amino acid sequence up to 9 amino acids directly upstream of the I/L/V-x-N/A/L motif of the native signal peptide sequence of FCb2; and iv. the I/L/V-x-N/A/L motif, which can be of number 0, 1, or can be partially truncated, wherein if the number is 0 or the I/L/V-x-N/A/L is partially truncated, the length of the amino acid sequence of iii. is 0, wherein the amino acid sequence of said recombinant FCb2 is different from SEQ ID NO: 145.

### FIELD OF THE INVENTION

The present invention relates to the field of recombinant enzymes, in particular flavocytochrome b2, and means of producing such enzymes in yeast cells. The invention specifically relates to recombinant flavocytochrome b2 proteins comprising a truncated N-terminus.

### BACKGROUND OF THE INVENTION

The detection and measurement of L-lactate is important in various fields. In the food and beverage industries, as well as clinical diagnostics, there is a need for highly-selective, sensitive, rapid, and reliable methods of determining the presence of key ingredients or metabolites which determine the quality of the product or serve as markers for diseases or the physiological state of humans. L-lactate is one of these metabolites. Also, analysis of lactate levels in blood is important in clinical diagnosis of hypoxia, lactic acidosis, and drug toxicity tests, hyperlactemia in diabetic and liver diseases, sepsis, and thiamine deficit. It is also measured when monitoring the performance of athletes and developing optimum training regimens.

The determination of lactate content is typically based on the enzymatic oxidation of L-lactate to pyruvate. Traditionally, the enzymatic methods are based on NAD+-dependent lactate dehydrogenases (LDH) isolated from animal muscles or heart, or on bacterial lactate oxidase (LOx) used in a chromogenic system. Many other methods have been proposed as well; for example, spectrophotometry, fluorimetry, pH potentiometric measurements, and amperometric biosensors based on O₂ consumption and H₂O₂ formation detected on electrodes. The actual lactate content is determined by the spectrophotometric detection of NADH or a colorimetric assay of H₂O₂.

In Kavita R. et al. (2016), biosensors based on electrochemical lactate detection are reviewed.

Flavocytochrome b2 (FCb2) are enzymes that are promising candidates for lactate DET (direct electron transfer) recognition elements. This enzyme is known for participating in the lactic acid metabolism in yeasts, and generally referred to as L-lactate-cytochrome c oxidoreductase (EC 1.1.2.3; flavocytochrome b2, FCb2, L-lactate cytochrome c oxidoreductase). FCb2 catalyzes the electron transfer from L-lactate to cytochrome c in yeast mitochondria.

In nature, FCb2 is located in the mitochondrial intermembrane space. The import of proteins into the mitochondrial intermembrane space is reviewed by Edwards R. et al. (2021). Thereby, it is described that FCb2 is imported into the mitochondrial intermembrane space by the stop-transfer pathway. FCb2 contains a bipartite signal which is composed of a N-terminal positively charged mitochondrial targeting signal (MTS) followed by a hydrophobic segment. The MTS is targeted through the translocon of the outer membrane (TOM) and also through the translocon of the inner membrane (TIM23) into the matrix via the presequence translocase-associated motor (PAM). Further translocation to the matrix is blocked when the stop-transfer hydrophobic signal enters TIM23 and causes translocational arrest followed by lateral diffusion of this segment into the inner membrane. The MTS is cleaved by the mitochondrial processing peptidase (MPP) and a mature IMS protein is released via a second cleavage event mediated by IMS proteases.

Beasley et al. (1993) studied the sorting signal of yeast FCb2 to the mitochondrial intermembrane space by fusing the first 167 residues of FCb2 to Cytochrome oxidase subunit IV (CoxIV) as a passenger protein. Thereby, mutations causing a mislocalization of CoxIV have been analyzed.

Hartl et al. (1987) investigated the targeting of cytochrome b2 and c1 to the intermembrane space. Both proteins are synthesized on cytoplasmic ribosomes and processed in mitochondria in two steps. First, the precursors are translocated across both mitochondrial membranes to the matrix where processing of matrix peptidase leads to intermediate-sized forms. The second proteolytic processing occurs in the intermembrane space. The authors conclude that the hydrophobic stretches in the bipartite signal peptide of the intermediate-sized forms of these proteins act as a transport signal to direct export from the matrix into the intermembrane space.

Koll et al. (1992) disclose the effect of hsp60 on the transport of FCb2 into the mitochondrial intermembrane space.

Campo et al. (2016) discuss mitochondrial mega-channels for the import of proteins and nucleic acids into mitochondria. Thereby, signal sequences and sorting pathways of mitochondrial proteins are described.

Black et al. (1989) disclose that the mature sequences of the two FCb2 enzymes from Hansenula anomala and Saccharomyces cerevisiae are strikingly similar. The N-terminal leader sequences of these enzymes share a significant similarity. It is presumed that the different regions of these leader sequences are functionally similar and that a number of relevant features are found. The N-terminus contains a high degree of basic amino acids which is a feature that almost all imported mitochondrial proteins have in common. As found in other polypeptides transported to the mitochondrial intermembrane space, there is an uncharged segment towards the C-terminal end of the leader sequence. A conserved feature indicating a common cleavage mechanism is the acidic nature of the first amino acid of the mature N-terminus.

EP3080283A2 discloses a truncated version of the Saccharomyces cerevisiae ILV2 gene encoding an acetolactate synthase, wherein the mitochondrial targeting signal is absent and a cytosolic form of acetolactate synthase is encoded by the gene. Naturally, the ILV2 protein is located in the mitochondrial matrix. Further described is a recombinant yeast strain comprising said gene encoding the cytosolic acetolactate synthase for enhanced glycerol production.

Presently, FCb2 is won by isolating the enzyme from yeast, e.g. S. cerevisiae or H. anomala. However, FCb2 from S. cerevisiae is unstable and the enzyme is difficult to isolate and purify, which makes it problematic to produce enough enzyme for its application e.g. in bioanalytical devices. FCb2 isolated from thermo-tolerant methylotrophic yeast Hansenula polymorpha was used previously as biological recognition element in amperometric biosensors (Smutok et al. 2013). There it was shown that the enzyme was capable of direct electron transfer. However, the stability of FCb2 isolated from H. polymorpha was also limited.

WO 2021/167011 A1 discloses a device for measuring lactic acid using lactate dehydrogenase.

A chimeric fusion protein comprising the heme domain of FCb2 from Pichia pastoris and an engineered L-lactate oxidase from Aerococcus viridans has been generated to produce a multiplexed DET-type lactate and glucose sensor. The fusion protein was expressed in E. coli (Hiraka et al. 2020).

Thus, there is an urgent need in the field for improved means of producing recombinant FCb2 in yeast and in particular FCb2 variants, that can be tailored to their use, e.g. as lactate DET recognition elements in enzymatic biosensors.

### SUMMARY OF THE INVENTION

It is the objective of the present invention to provide improved means for the production of recombinant flavocytochrome b2 enzymes.

The objective is solved by the subject matter of the present invention.

The present invention relates to recombinant flavocytochrome b2 (FCb2) comprising a truncated N-terminal signal peptide sequence. The inventors of the present invention have surprisingly discovered that yeast FCb2 comprising a truncated signal peptide sequence can be produced with significantly improved yield and displays significantly improved activity.

According to the invention there is provided a recombinant flavocytochrome b2 (FCb2), comprising a mature FCb2 peptide sequence of a native FCb2, or a functionally active variant of said mature FCb2 peptide sequence, wherein the N-terminus of said mature FCb2 peptide sequence consists of a truncated signal peptide sequence replacing the native signal peptide sequence, and wherein said truncated signal peptide sequence is of the following structure from N- to C-terminus:
i. a methionine;
ii. optionally a tag sequence;
iii. an amino acid sequence of a length of 0, 1, 2, 3, 4, 5, 6, 7, 8, 9 amino acids of the native signal peptide sequence, corresponding to the amino acid sequence up to 9 amino acids directly upstream of the I/L/V-x-N/A/L motif of the native signal peptide sequence of FCb2; and
iv. the I/L/V-x-N/A/L motif, which can be of number 0, 1, or can be partially truncated, wherein if the number is 0 or the I/L/V-x-N/A/L is partially truncated, the length of the amino acid sequence of iii. is 0,
wherein the amino acid sequence of said recombinant FCb2 is different from SEQ ID NO: 145.

The physiological role of FCb2 in yeast relies on its presence in the mitochondrial transmembrane space. After or during expression in the endoplasmic reticulum (and cellular trafficking), the FCb2 is thought to undergo maturation during the incorporation into the mitochondrial membrane, similar to cellular secretion assisted by a signal peptide. In the process of incorporating the enzyme into the mitochondria, the FCb2 is truncated at the N-terminus, and the mature, shorter enzyme is then found in the mitochondria. This process is referred to as co-translational translocation. Analysis of peptide sequences of characterized and uncharacterized putative FCb2 genes by the inventors identified several sequence patterns in the N-terminal pre-sequence of FCb2, indicating that there could be N-terminal pre-sequences located upstream of the mature FCb2 sequence that have not been previously identified and are putatively responsible for translocation of FCb2 into the mitochondrial intermembrane space.

A multiple sequence alignment revealed that yeast FCb2 sequences share an I/L/V-x-N/A/L motif in the N-terminal pre-sequence, which sequence is also herein referred to as “signal peptide sequence”, “translocation peptide sequence” or “translocation sequence”. Surprisingly, deletion of the signal peptide sequence, except for the N-terminal methionine, up to or even including the I/L/V-x-N/A/L motif provides for significantly improved production of recombinant FCb2 in yeast. Specifically, recombinant FCb2 comprising such a truncated signal peptide sequence can be produced with very high activity and at high yields, making use of the protein processing and cofactor synthesis capabilities of the original FCb2 host, yeasts. Thereby, the expression of a functionally active FCb2 in the cytosol is enabled due to the specific signal peptide sequence-truncation pattern of the invention. Surprisingly, the inventors found that in order to successfully express a functionally active FCb2 in the cytosol, the deletion of the N-terminal region of the signal peptide sequence known to be responsible for the translocation of the FCb2 into the mitochondria is not enough. Instead, the inventors surprisingly found that the signal peptide has to be even more truncated according to a specific pattern for successful cytosolic expression of active FCb2. This truncation pattern is based on the highly conserved I/L/V-x-N/A/L motif among yeast FCb2 sequences. Specifically, the recombinant FCb2 of the present invention comprises or contains the mature FCb2 sequence derived from a native yeast FCb2 sequence and a truncated signal peptide sequence, wherein the sequence of the truncated signal peptide corresponds to the sequence of the native signal peptide of the yeast FCb2 but comprises a truncation as described herein. Specifically said truncation is a deletion of the native signal peptide sequence up to or including the I/L/V-x-N/A/L motif.

Specifically, the truncated signal peptide sequence described herein comprises an N-terminal methionine, the I/L/V-x-N/A/L motif and either none of the amino acids N-terminal of said motif, or up to 9 of the amino acids N-terminal of said motif in the respective native signal peptide sequence and none of the amino acids upstream of said up to 9 amino acids. Specifically, the truncated signal peptide sequence comprises up to 1, 2, 3, 4, 5, 6, 7, 8, or 9 of the amino acids N-terminal of the I/L/V-x-N/A/L motif and an N-terminal methionine.

Specifically, the mature FCb2 peptide sequence is the sequence of an FCb2 peptide as it is naturally found in the yeast mitochondrion, specifically in the mitochondrial intermembrane space. Specifically, the recombinant FCb2 described herein comprises a sequence based on the mature form of FCb2 naturally found in the yeast mitochondrial intermembrane space, which comprises a cytochrome b2 domain, a flavin domain, a hinge region connecting the cytochrome b2 domain and the flavin domain and a tail region at its C-terminus.

Specifically, the recombinant FCb2 described herein comprises a mature FCb2 peptide sequence comprising at least a yeast heme domain and a yeast flavin domain.

According to a specific example, the mature FCb2 peptide sequence of the recombinant FCb2 described herein comprises or consists of SEQ ID NO:1 or SEQ ID NO:2.

In a specific embodiment, the mature FCb2 peptide sequence of the recombinant FCb2 described herein is a functionally active variant of a native FCb2 mature peptide sequence found in the yeast mitochondrial intermembrane space and comprises one or more point mutations in the nucleotide sequence encoding the mature FCb2 sequence, compared to the respective native mature FCb2 sequence. Specifically, it comprises 1, 2, 3, 4, 5, 6, 7, 8, 9, 10, 11, 12, 13, 14, 15, 16, 17, 18, 19 or 20, point mutations, specifically resulting in one or more amino acid substitutions, additions or deletions, or the like. Specifically, the functional variant of the native mature FCb2 peptide sequence is a full-length mature FCb2 peptide sequence comprising point mutations, or it is a fragment of the full-length mature FCb2 peptide sequence with retained enzymatic activity. Specifically, a variant of a mature FCb2 sequence is functionally active if it is capable of converting L-lactate to pyruvate. Specifically, a functionally active variant of a mature FCb2 sequence has at least 10, 10, 20, 30, 40, 50, 60, 70, or even more % of the enzymatic activity of the corresponding wild type mature FCb2 sequence. Specifically, a functionally active variant of a mature FCb2 sequence has at least 10, 20, 30, 40, 50, 60, 70, or even more % of the enzymatic activity of the corresponding wild type mature FCb2 sequence, wherein said enzymatic activity is determined with the CytC assay and L-lactate as substrate.

The enzymatic activity of a flavocytochrome b2 variant can be readily determined by assays known in the art, such as assays determining the colorimetric reduction of cytochrome c, ferricyanide or 2,6-dichloroindophenol (DCIP). Specifically, the recombinant FCb2 described herein comprises an enzymatic activity of at least 1 U/mg as determined by the CytC assay described by Diêp Lê et al. (Diêp Lê et al. 2009).

Specifically, the mature FCb2 peptide sequence described herein comprises SEQ ID NO:1 or SEQ ID NO:2 or at least 60%, preferably at least 70%, at least 80%, more preferably at least 90% sequence identity to SEQ ID NO:1 or SEQ ID NO:2. Specifically, the mature FCb2 peptide sequence comprises at least about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% sequence identity to SEQ ID NO:1 or SEQ ID NO:2.

According to a specific embodiment, the mature FCb2 peptide sequence is derived from Saccharomyces cerevisiae FCb2 (SEQ ID NO: 3) and the truncated signal peptide sequence including the N-terminal methionine is selected from the group consisting of SEQ ID NOs:37, 38, and 39. Specifically, the recombinant FCb2 described herein comprises or consists of a sequence selected from the group consisting of SEQ ID NOs:4, 5, and 6. Specifically, the recombinant FCb2 of the present invention comprising a sequence selected from the group consisting of SEQ ID NOs:4, 5 and 6 further comprises an N-terminal His tag, preferably a 6-His or 8-His tag.

According to a further specific embodiment, the mature FCb2 peptide sequence is derived from Kluyveromyces marxianus FCb2 (SEQ ID NO: 7) and the truncated signal peptide sequence including the N-terminal methionine is selected from the group consisting of SEQ ID NOs:40, 41 and 42. Specifically, the recombinant FCb2 described herein comprises or consists of a sequence selected from the group consisting of SEQ ID NOs:8, 9 and 10. Specifically, the recombinant FCb2 of the present invention comprising a sequence selected from the group consisting of SEQ ID NOs:8, 9 and 10 further comprises an N-terminal His tag, preferably a 6-His or 8-His tag.

According to yet a further specific embodiment, the mature FCb2 peptide sequence is derived from Wickerhamomyces anomalus FCb2 (SEQ ID NO:11) and the truncated signal peptide sequence including the N-terminal methionine is SEQ ID NO:43 or 44 or it is M S A (Met-Ser-Ala). Specifically, the recombinant FCb2 described herein comprises or consists of a sequence selected from the group consisting of SEQ ID NOs:12, 13, and 14. Specifically, the recombinant FCb2 of the present invention comprising a sequence selected from the group consisting of SEQ ID NOs:8-10 further comprises an N-terminal His tag, preferably a 6-His or 8-His tag.

Specifically, the recombinant FCb2 described herein comprises a truncated signal peptide sequence as described herein and a corresponding mature FCb2 peptide sequence, which are derived from S. cerevisiae, W. anomalus, K. marxianus, O. parapolymorpha, Candida glabrata, Kluyveromyces lactis, Lachancea thermotolerans, Saccharomycodes ludwigii, Naumovozyma castelli, Zygosaccharomyces baidii, Zygosaccharomyces parabalii, Lachancea mirantina, Tetrapisispora phaffii, Saccharomyces eubayanus, Saccharomyces kudriavzevii, Saccharomyces paradoxus, Vanderwaltozyma polyspora, Lachancea dasiensis, Wickerhamomyces ciferri, Kluyveromyces dobzhanskii, Kazachstania naganishii, Zygosaccharomyces mellis, Kazachstania saulgeensis, Candida boidinii, Lachancea fermentati, Zygosaccharomyces rouxii, Cyberlindnera fabianii, Cyberlindnera jadinii, Kazachstania africana, Lachancea quebecensis, Kuraishia capsulata, Torulaspora delbrueckii, Komogatella pastoris, Komagatella phaffii, Lachancea nothofagi, or Naumovomyces dairenensis.

Specifically, the recombinant FCb2 described herein comprises a mature FCb2 peptide sequence derived from the FCb2 of Saccharomyces cerevisiae, Kluyveromyces marxianus, Wickerhamomyces anomalus, Naumovozyma castelli or Cyberlindera fabianii.

In a specific embodiment, the recombinant FCb2 described herein comprises a tag sequence. Tag sequences, such as e.g. poly-histidine tags, are helpful tools in recombinant protein production. Full-length FCb2 is difficult to express with a tag sequence such as an N-terminal poly-his tag. Surprisingly, recombinant FCb2 comprising a truncated signal peptide sequence as described herein can be expressed with an N-terminal tag, which significantly improves and simplifies its production process.

Specifically, the tag sequence is selected from the group consisting of affinity tags, solubility enhancement tags and monitoring tags. Specifically, the tag sequence is an N-terminal tag.

Specifically, the affinity tag is selected from the group consisting of poly-histidine (poly H) tag, poly-arginine (poly A) tag, FLAG tag, Strep tag, streptavidin-binding peptide (SBP) tag, calmodulin-binding peptide (CBP) tag, S-tag, HA tag, c-Myc tag, and SUMO tag, specifically the tag is a His tag comprising one or more His, more specifically it is a hexahistidine- or 8-His tag.

Specifically, the solubility enhancement tag is selected from the group consisting of T7A, T7A1, T7A2, T7A3, T7A4, T7A5, T7B, T7B1, T7B2, T7B3, T7B3, T7B4, T7B5, T7B6, T7B6, T7B7, T7B8, T7B9, T7B10, T7B11, T7B12, T7B13, T7C, calmodulin-binding peptide (CBP) tag, poly A tag, poly Lys tag, protein D tag (dTAG), Z domain of Staphylococcal protein A, and thioredoxin.

Specifically, the monitoring tag is selected from the group consisting of m-Cherry, GFP, and f-Actin.

According to a specific example, the recombinant FCb2 described herein has an amino acid sequence selected from SEQ ID NO:4, 5, 6, 8, 9, 10, 12, 13, 14, 24, 25, 26, 27, 28, 29, 30, 31, 32, 33, 34, 35, and 36.

Further provided herein is an electrode comprising a recombinant flavocytochrome b2 (FCb2) comprising a mature FCb2 peptide sequence of a native FCb2, or a functionally active variant of said mature FCb2 peptide sequence, with an N-terminus consisting of a truncated signal peptide sequence replacing the native signal peptide sequence, wherein said truncated signal peptide sequence is of the following structure from N- to C-terminus:
i. a methionine;
ii. optionally a tag sequence;
iii. an amino acid sequence of a length of 0, 1, 2, 3, 4, 5, 6, 7, 8, 9 amino acids of the native signal peptide sequence, corresponding to the amino acid sequence up to 9 amino acids directly upstream of the I/L/V-x-N/AIL motif of the native signal peptide sequence of FCb2; and
iv. the I/L/V-x-N/A/L motif, which can be of number 0, 1, or can be partially truncated, wherein if the number is 0 or the I/L/V-x-N/A/L is partially truncated, the length of the amino acid sequence of iii. is 0.

Specifically, the electrode comprising a recombinant FCb2 can be used for determining L-lactate in a sample. Specifically, the electrode comprising a recombinant FCb2 can be used as lactate DET recognition elements in enzymatic biosensors.

Further provided herein is a method of producing a recombinant flavocytochrome b2 (FCb2), comprising the steps of:
i. providing a yeast cell comprising a promoter functional in yeast operably linked to a recombinant nucleic acid sequence encoding an FCb2, wherein the native signal peptide sequence of said FCb2 has been replaced with a truncated signal peptide sequence consisting of the following structure from N- to C-terminus:
   a) a methionine;
   b) optionally a tag sequence;
   c) an amino acid sequence of a length of 0, 1, 2, 3, 4, 5, 6, 7, 8, 9 amino acids of the native signal peptide sequence, corresponding to the amino acid sequence up to 9 amino acids directly upstream of the I/L/V-x-N/A/L motif of the native signal peptide sequence of FCb2; and
   d) the I/L/V-x-N/A/L motif, which can be of number 0, 1, or can be partially truncated, wherein if the number is 0 or the I/L/V-x-N/A/L is partially truncated, the length of the amino acid sequence of c) is 0; and
ii. cultivating said yeast cell in a medium conducive for the expression of said FCb2.

In a specific embodiment of the method provided herein, the recombinant nucleic acid sequence encoding said recombinant FCb2 is stably integrated into the yeast cell's genome.

Specifically, the yeast cells are methylotrophic yeast cells, preferably Pichia pastoris (Komagataella phaffii), Kluyveromyces lactis, or non-methylotrophic yeast cells, preferably Saccharomyces cerevisiae cells.

Specifically, the nucleic acid sequence encoding the FCb2 is derived from S. cerevisiae, W. anomalus, K. marxianus, O. parapolymorpha, Lachancea thermotolerans, Saccharomycodes ludwigii, Naumovozyma castelli, Zygosaccharomyces bailii, Zygosaccharomyces parabalii, Lachancea mirantina, Tetrapisispora phaffii, Saccharomyces eubayanus, Saccharomyces kudriavzevii, Saccharomyces paradoxus, Vanderwaltozyma polyspora, Candida glabrata, Kluyveromyces lactis, Lachancea dasiensis, Wickerhamomyces ciferri, Kluyveromyces dobzhanskii, Kazachstania naganishii, Zygosaccharomyces mellis, Kazachstania saulgeensis, Candida boidinii, Lachancea fermentati, Zygosaccharomyces rouxii, Cyberlindnera fabianii, Cyberlindnera jadinii, Kazachstania africana, Lachancea quebecensis, Kuraishia capsulata, Torulaspora delbrueckii, Komogatella pastoris, Komagatella phaffii, Lachancea nothofagi, or Naumovomyces dairenensis.

Specifically, the mature FCb2 peptide sequence is derived from S. cerevisiae FCb2 and the truncated signal peptide sequence is selected from the group consisting of SEQ ID NOs:37-39.

Specifically, the mature FCb2 peptide sequence is derived from K. marxianus FCb2 and the truncated signal peptide sequence is selected from the group consisting of SEQ ID NOs:40-42.

Specifically, the mature FCb2 peptide sequence is derived from W. anomalus FCb2 and the truncated signal peptide sequence is SEQ ID NO:43 or 44 or it is of the amino acid sequence M S A (Met-Ser-Ala).

Specifically, the recombinant FCb2 produced according to the method provided herein comprises an N-terminal tag sequence, preferably selected from the group consisting of affinity tags, solubility enhancement tags and monitoring tags, and most preferably a poly-histidine tag.

Specifically, the recombinant FCb2 produced according to the method provided herein has a sequence selected from the group consisting of SEQ ID NOs: 4, 5, 6, 8, 9, 10, 12, 13, 14, 33, 34, 35, and 36.

Specifically, the promoter is a methanol-inducible promoter and the medium conducive for the expression of the recombinant FCb2 comprises methanol.

Specifically, the method for the production of recombinant FCb2 as described herein further comprises the step of isolating the FCb2 from the yeast cell, preferably by disrupting the yeast cells to release the intracellular FCb2 into the medium followed by purifying the FCb2 from the medium.

Specifically, the yeast cells used in the method provided herein are methylotrophic yeast cells selected from the group consisting of Pichia pastoris, Hansenula polymorpha, Pichia minuta, Candida boidinii, or yeast cells selected from the group of non-methylotrophic yeasts consisting of Saccharomyces cerevisiae, Kluyveromyces lactis, Yarrowia lipolytica, Arxula adeninivorans, Zygosaccharomyces bailii, Pichia stipites, Kluyveromyces marxianus, Saccharomyces occidentalis, Zygosaccharomyces rouxii, preferably the yeast cells are Saccharomyces cerevisiae, Kluyveromyces lactis, or Pichia pastoris cells, most preferably the yeast cells are Pichia pastoris cells.

Further provided herein is a nucleic acid molecule encoding the recombinant FCb2 described herein. Specifically, the nucleic acid molecule encoding the recombinant FCb2 as described herein is different from a nucleic acid molecule encoding the amino acid sequence given in SEQ ID NO: 145. Further provided herein is an expression cassette comprising the recombinant FCb2 described herein operably linked to regulatory elements. Specifically provided is an expression cassette comprising a promoter, a sequence encoding the recombinant FCb2 described herein and a terminator sequence.

Further provided herein is a yeast host cell or a yeast host cell line expressing the recombinant FCb2 described herein.

Further provided herein is a yeast host cell comprising a nucleic acid molecule encoding a mature FCb2 peptide sequence of a native FCb2, or a functionally active variant of said mature FCb2 peptide sequence, with an N-terminus consisting of a truncated signal peptide sequence replacing the native signal peptide sequence, wherein said truncated signal peptide sequence is of the following structure from N- to C-terminus:
i. a methionine;
ii. optionally a tag sequence;
iii. an amino acid sequence of a length of 0-9 amino acids of the native signal peptide sequence, corresponding to the amino acid sequence up to 9 amino acids directly upstream of the I/L/V-x-N/A/L motif of the native signal peptide sequence of FCb2; and
iv. the I/L/V-x-N/A/L motif, which can be of number 0, 1, or can be partially truncated, wherein if the number is 0 or the I/L/V-x-N/A/L is partially truncated, the length of the amino acid sequence of iii. is 0.

Specifically, the host cells are selected from the group of methylotrophic yeasts consisting of Pichia pastoris, Hansenula polymorpha, Pichia minuta, Candida boidinii, or yeast cells selected from the group of non-methylotrophic yeasts consisting of Saccharomyces cerevisiae, Kluyveromyces lactis, Yarrowia lipolytica, Arxula adeninivorans, Zygosaccharomyces bailii, Pichia stipites, Kluyveromyces marxianus, Saccharomyces occidentalis, Zygosaccharomyces rouxii, preferably the host cells are Saccharomyces cerevisiae or Pichia pastoris cells.

### FIGURES

FIG. 1. Mitochondrial cleavage site prediction for ScFCb2 (TargetP)

FIG. 2. Mitochondrial cleavage site prediction for KmFCb2 (TargetP)

FIG. 3. Mitochondrial cleavage site prediction for WaFCb2 (TargetP)

FIG. 4. Mitochondrial cleavage site prediction for CangFCb2 (TargetP)

FIG. 5. Multiple Sequence Alignment of FCb2 N′-terminal sequences from phylogenetic data, curated to the family of budding yeasts Saccharomycetaceae.

FIG. 6. Multiple sequence alignment with WebLogo (“1 FCB” is the reference sequence from the cleaved and matured ScFCb2 obtained from the resolved crystal structure, PDB: 1FCB)

FIG. 7. ClustalOmega multiple sequence alignment. Underlined: Sequence beginning of experimentally confirmed cleaved and maturated N-terminus of S. cerevisiae and W. anomalus FCb2. Grey: Discovered TransP recognition motif. “*”, “:” and “.” in the subtext indicate different degrees of conservation of structurally relevant sequence parts from good to worse.

FIG. 8. (A) Volumetric activities from screening of ScFCb2 TransP constructs. (B) Data summary from activity screening (CytC assay) of ScFCb2 TransP constructs. (C) N-terminal TransP sequence with TransP recognition motif (grey) and relative activities.

FIG. 9. (A) Volumetric activities from screening of KmFCb2 TransP constructs. (B) Data summary from activity screening (CytC assay) of KmFCb2 TransP constructs. (C) N-terminal TransP sequence with TransP recognition motif (grey) and relative activities.

FIG. 10. (A) Volumetric activities from screening of KmFCb2 TransP constructs. (B) Data summary from activity screening (CytC assay) of KmFCb2 TransP constructs. (C) N-terminal TransP sequence with TransP recognition motif (grey) and relative activities.

FIG. 11. Obtained activities after purification in terms of (A) enzymatic units from CytC assay [U/g cell pellet] and (B) purified enzyme in terms of amount from optical absorbance [mg enzyme/g cell pellet] (C) SDS PAGE of purified his-tagged TransP-His FCb2 constructs.

FIG. 12. Amino acid sequences referred to herein.

FIG. 13. Calibration of FCb2 modified carbon paste electrodes at 37° C. and an applied potential of +0.2 V by adding increasing lactate concentrations over time. WaFCb2 (A), KmFCb2 (D), and CangFCb2 (G): Stepwise sensor signal increase after stepwise increase of lactate. WaFCb2 (B), KmFCb2 (E), and CangFCb2 (H): Decrease of sensor signals over time in presence of 30 mM lactate. WaFCb2 (C), KmFCb2 (F), and CangFCb2 (I): Dependence of sensor current densities on lactate concentration. Enzyme load: 0.8 μg/mm² FIG. 14. Comparison of current densities of 0.8 μg/mm² KmFCb2 per carbon paste electrodes depending on the pre-treatment of electrodes. Calibration of FCb2 modified carbon paste electrodes at 37° C. and an applied potential of +0.2 V by adding increasing lactate concentrations over time. A: CTAB; B: EGDGE; C: no pre-treatment.

FIG. 15. Response of a KmFCb2 modified gold or carbon paste electrode at 37° C. and an applied potential of +0.2 V after adding 30 mM lactate. A: Gold—no pre-treatment; B: Carbon paste—no pre-treatment.

FIG. 16: Temperature stability of KmFCb2 (A); Heat capacity curve of KmFCb2 (B) FIG. 17: Temperature stability of CangFCb2 (A); Heat capacity curve of CangFCb2 (B) FIG. 18: SDS PAGE of FCb2 expressed in E. coli compared to FCb2 expressed in Pichia pastoris

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (4th Ed.), Vols. 1-3, Cold Spring Harbor Laboratory Press (2012); Krebs et al., “Lewin's Genes XI”, Jones & Bartlett Learning, (2017); Berg et al, “Stryer Biochemie” Springer Verlag, 2018; and Murphy & Weaver, “Janeway's Immunobiology” (9th Ed., or more recent editions), Taylor & Francis Inc, 2017.

The subject matter of the claims specifically refers to artificial products or methods employing or producing such artificial products, which may be variants of native (wild-type) products. Though there can be a certain degree of sequence identity to the native structure, it is well understood that the materials, methods and uses of the invention, e.g., specifically referring to isolated nucleic acid sequences, amino acid sequences, expression constructs, transformed host cells and modified proteins and enzymes, are “man-made” or synthetic, and are therefore not considered as a result of “laws of nature”.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

As used herein and in the claims, the singular form, for example “a”, “an” and “the” includes the plural, unless the context clearly dictates otherwise.

As used herein, amino acids refer to twenty naturally occurring amino acids encoded by sixty-one triplet codons. These 20 amino acids can be split into those that have neutral charges, positive charges, and negative charges:

The “neutral” amino acids are shown below along with their respective three-letter and single-letter code and polarity: Alanine(Ala, A; nonpolar, neutral), Asparagine (Asn, N; polar, neutral), Cysteine (Cys, C; nonpolar, neutral), Glutamine (Gln, Q; polar, neutral), Glycine (Gly, G; nonpolar, neutral), Isoleucine (lie, I; nonpolar, neutral), Leucine (Leu, L; nonpolar, neutral), Methionine (Met, M; nonpolar, neutral), Phenylalanine (Phe, F; nonpolar, neutral), Proline (Pro, P; nonpolar, neutral), Serine (Ser, S; polar, neutral), Threonine (Thr, T; polar, neutral), Tryptophan (Trp, W; nonpolar, neutral), Tyrosine (Tyr, Y; polar, neutral), Valine (Val, V; nonpolar, neutral), and Histidine (His, H; polar, positive (10%) neutral (90%)).

The “positively” charged amino acids are: Arginine (Arg, R; polar, positive), and Lysine (Lys, K; polar, positive).

The “negatively” charged amino acids are: Aspartic acid (Asp, D; polar, negative), and Glutamic acid (Glu, E; polar, negative). The term “enzyme” in accordance with the invention means any substance composed wholly or largely of protein or polypeptides that catalyzes or promotes, more or less specifically, one or more chemical or biochemical reactions. Specifically, the term “enzyme” is used herein to refer to a protein or polypeptide capable of converting L-lactate to pyruvate.

In humans, pyruvate is converted to lactate by lactate dehydrogenase (“LDH”).

In yeast, L-lactate is converted to pyruvate by L-lactate cytochrome c-oxidoreductase (EC 1.1.2.3), which is herein referred to as “Flavocytochrome b2” or “FCb2”.

The term “lactate” used herein refers to a lactic acid or a salt thereof.

The physiological role of FCb2 in yeast relies on its presence in the mitochondrial transmembrane space. After or during expression in the endoplasmic reticulum (and cellular trafficking), the FCb2 is thought to undergo maturation during the incorporation into the mitochondrial membrane, which is assisted by signal peptide recognition.

Multiple sequence alignment of characterized and putative FCb2 sequences revealed that yeast FCb2 sequences share an I/L/V-x-N/A/L motif in the N-terminal pre-sequence, which N-terminal pre-sequence is also herein referred to as “signal peptide sequence”, “signal sequence” or “translocation peptide sequence” or “translocation sequence”. Surprisingly, deletion of the signal peptide sequence of FCb2 up to or even including the I/L/V-x-N/A/L motif allows for significantly improved production of recombinant FCb2. Specifically, recombinant FCb2 comprising such a truncated signal peptide sequence can be produced at high yields and with significantly improved activity.

Native yeast flavocytochrome b2 (FCb2) has two functional domains that are connected via a “hinge” linker (57 kDa monomer). The FCb2 from S. cerevisiae is the best studied representative and has been crystallized (PDB 1 FCB). The following sections have been identified in the FCb2 of S. cerevisiae:
N-terminal pre-sequence, up to amino acid residue 88 of SEQ ID NO:3. This so-called “translocation peptide” is cleaved upon translocation;
Cytochrome-b2 domain: <10 kDa domain with non-covalent heme b: (spanning amino acid residues 88-165 of SEQ ID NO:3);
Hinge region with domain-connecting linker (spanning residues 177-190 of SEQ ID NO:3), connecting the cytochrome b2 domain and the flavin domain;
Flavin domain of approx. 45 kDa, containing a flavin mononucleotide (FMN) (spanning residues 197-563 of SEQ ID NO:3);
Tail region (comprising residues 567-591 of SEQ ID NO:3), extends at the C-terminus and wraps around dimers.

The recombinant FCb2 of the present invention comprises at least a yeast cytochrome b2 domain and a yeast flavin domain, which may be directly connected or linked via a hinge region. Specifically, the recombinant FCb2 described herein also comprises a C-terminal tail region. Specifically, the recombinant FCb2 described herein comprises a sequence based on the mature form of FCb2 naturally found in the yeast mitochondrial intermembrane space, which comprises a cytochrome b2 domain, a flavin domain, a hinge region connecting the cytochrome b2 domain and the flavin domain and a tail region at its C-terminus.

The recombinant FCb2 of the present invention further comprises a truncated signal peptide sequence, wherein the sequence of the truncated signal peptide corresponds to the signal of the native signal peptide but comprises a truncation as described herein.

In a specific embodiment, the recombinant FCb2 of the present invention comprises all of the above-identified FCb2 S. cerevisiae sections, or its homologues from a different yeast, except for the N-terminal pre-sequence which is replaced with a truncated signal peptide sequence as described herein.

As used herein, the term “signal peptide sequence” refers to the N-terminal pre-sequence of FCb2. The signal peptide sequence is the sequence of the FCb2 protein that is cleaved off following translocation of the enzyme into the mitochondrion. Upon cleavage of the native signal peptide sequence, the mature FCb2 sequence remains and is functional in its physiological role in the mitochondria. The mature sequence of native FCb2 can be readily identified by a person skilled in the art, e.g. by isolating FCb2 from a yeast cell and sequencing the isolated protein. The signal peptide sequence and the mature sequence of a yeast FCb2 may also be determined using other routine methods known in the art, such as crystallography.

Thereby, the term “native” as used herein refers to the genetically encoded and unprocessed amino acid sequence as it occurs in nature before transport into the mitochondrial intermembrane space. Thus, the native amino acid sequence refers to the amino acid sequence before the native signal peptide sequence is cleaved off the native amino acid sequence which results in the mature amino acid sequence. For example, the native amino acid sequence of the full length FCb2 from Saccharomyces cerevisiae is given in SEQ ID NO: 3. Thereby, the amino acids 1 to 80 in SEQ ID NO: 3 are the native signal peptide sequence. The sequence of the mature FCb2 is given in SEQ ID NO: 1, wherein this sequence does not comprise amino acids 1 to 80 in SEQ ID NO: 3. As can be seen from the example of FCb2, the sequence of the mature FCb2 given in SEQ ID NO: 1 does not comprise a methionine at the N-terminus since the native N-terminus is cleaved off from the amino acid sequence during transport into the mitochondrial intermembrane space.

As used herein, the term “truncated signal peptide sequence” refers to the signal peptide sequence of FCb2 which comprises a deletion at its N-terminus. As described herein, the recombinant FCb2 of the present invention comprises a truncated signal peptide sequence, wherein the sequence of the truncated signal peptide corresponds to the sequence of the native signal peptide but comprises a deletion of the native signal peptide sequence starting from the native N-terminus and stretching to or including the I/L/V-x-N/A/L motif. Thus, in one specific example, the recombinant FCb2 described herein comprises a deletion of the entire signal peptide sequence including the I/L/V-x-N/A/L motif.

The recombinant FCb2 described herein further comprises a methionine at its N-terminus and thus N-terminal of the truncated signal peptide sequence. Since a mature FCb2 found in nature does not have an N-terminal methionine, the recombinant FCb2 of the invention can be readily differentiated from a mature FCb2 of a native FCb2 as the recombinant FCb2 of the invention has a N-terminal methionine.

Protein synthesis is initiated by methionine in eukaryotes. The ribosome starts translation, the assembly of a protein out of amino acids, when it encounters the start codon in the mRNA, which is the sequence AUG. AUG codes for the amino acid methionine, and so protein translation of the FCb2 described herein begins with methionine.

According to another specific example, the truncated signal peptide sequence of the recombinant FCb2 described herein still comprises the I/L/V-x-N/A/L motif but comprises none of the amino acids N-terminal of said motif, i.e. all amino acids N-terminal of the I/L/V-x-N/A/L motif are deleted in the truncated signal peptide sequence, except for the N-terminal methionine.

According to yet another specific example, the truncated signal peptide sequence of the recombinant FCb2 described herein comprises up to 9 of the amino acids N-terminal of said I/L/V-x-N/A/L motif but none of the amino acids upstream of said up to 9 amino acids, except for the N-terminal methionine. Specifically, the truncated signal peptide sequence comprises up to 1, 2, 3, 4, 5, 6, 7, 8, or 9 of the amino acids N-terminal of the I/L/V-x-N/A/L motif and an N-terminal methionine.

As used herein, the term “I/L/V-x-N/A/L motif” refers to a conserved sequence of 3 amino acids in the amino acid sequence of the signal peptide sequence of FCb2. The amino acid at position 1 of said motif is any one of Isoleucine (I), Leucine (L) and Valine (V), followed by any amino acid at position 2 (X), and followed by Asparagine (N), Alanine (A) or Leucine (L) at position 3.

For example, the signal peptide sequence of the FCb2 of S. cerevisiae ranges from amino acids 1 to 80 of SEQ ID NO:3 and comprises SEQ ID NO:15. The I/L/V-x-N/A/L motif in the signal peptide sequence of the FCb2 of S. cerevisiae has the amino acid sequence I D N and is located at position 78-80 of SEQ ID NO:3.

As used herein, the term “mature FCb2 peptide sequence” refers to the sequence of the yeast FCb2 peptide as it is naturally found in the yeast mitochondrion, specifically in the mitochondrial intermembrane space. As described herein, the recombinant FCb2 provided herein comprises a mature FCb2 peptide sequence. The mature FCb2 peptide sequence of the recombinant FCb2 described herein may be the full-length wild type mature sequence, which is generated by specific cleavage steps of the translocation peptide upon translocation of FCb2 into the yeast mitochondrion intermembrane space. Since FCb2 is naturally present in the mitochondrial intermembrane space, the signal peptide of FCb2 is cleaved twice until final localization in the mitochondrial intermembrane space. Therefore, cleavage of said translocation peptide finally results in a defined N-terminus of the mature FCb2. Therefore, the term “mature FCb2 peptide sequence” specifically refers to a FCb2 sequence with the defined N-terminus generated by cleavage of specific peptidases during translocation of FCb2 into the mitochondrial intermembrane space. The person skilled in the art is able to determine the mature FCb2 by using specific tools for the determination of cleavage sites. For example, the skilled person can predict the N-terminus of the mature protein by comparing the FCb2 sequence of choice with the FCb2 sequences provided herein through sequence alignment and determination of the amino acid of said FCb2 corresponding to the amino acid of the N-terminus of a FCb2 disclosed herein. Specifically, the skilled person can perform a multiple sequence alignment of the sequences of full length FCb2 sequences provided herein in SEQ ID NO: 3 (native, full length FCb2 from Saccharomyces cerevisiae), SEQ ID NO:7 (native, full-length FCb2 from Kluyveromyces marxianus), SEQ ID NO:11 (native, full-length FCb2 from Wickerhamomyces anomalus), SEQ ID NO:16 (native, full-length FCb2 from Candida glabrata), and the full length sequence of a FCb2 of choice, search in the alignment for the TransP recognition motif I/L/V-x-N/A/L and the mature N-terminal sequence of said FCb2 sequences provided herein, and determine the corresponding N-terminus of the mature FCb2 of choice. For example, a method for performing a multiple sequence alignment is ClustalOmega provided by EMBL's European Bioinformatics Institute.

In a specific embodiment of the invention, the mature FCb2 sequence is the sequence of a native FCb2 sequence having one of the following N-termini (the first five amino acids of the sequence from N- to C-terminus are given, it is to be understood that the following sequences reflect only the N-terminus of the complete mature FCb2 sequence and that the mature FCb2 amino acid sequence merges into the rest of the respective sequence):

(SEQ ID NO: 157)
DAAKH,
(SEQ ID NO: 158)
TSSVL,
(SEQ ID NO: 159)
DVKLE,
(SEQ ID NO: 160)
GTTKD,
(SEQ ID NO: 161)
NELRD,
(SEQ ID NO: 162)
DSMLL,
(SEQ ID NO: 163)
DPKLD,
(SEQ ID NO: 164)
EPKLD,
(SEQ ID NO: 165)
DVSIE,
(SEQ ID NO: 166)
DAKFD,
(SEQ ID NO: 167)
GAKVD,
(SEQ ID NO: 168)
DLVRD,
(SEQ ID NO: 169)
DVPKW,
(SEQ ID NO: 170)
GVKVD,
(SEQ ID NO: 171)
ATKEE,
(SEQ ID NO: 172)
ESAQQ,
(SEQ ID NO: 173)
GTKVD,
(SEQ ID NO: 174)
DNGQP,
(SEQ ID NO: 175)
ESKLA,
(SEQ ID NO: 176)
DSSSS,
(SEQ ID NO: 177)
GPVKD,
(SEQ ID NO: 178)
GPKLD,
(SEQ ID NO: 179)
DVPHW,
(SEQ ID NO: 180)
DAPTQ,
(SEQ ID NO: 181)
EAITT,
(SEQ ID NO: 182)
ESPSV,
(SEQ ID NO: 183)
DAAFD,
(SEQ ID NO: 184)
DIKYE,
(SEQ ID NO: 185)
GTVKD,
(SEQ ID NO: 186)
DDERK,
(SEQ ID NO: 187)
DVRQD,
(SEQ ID NO: 188)
DSKME.

According to another embodiment of the invention, the mature FCb2 peptide sequence of the recombinant FCb2 described herein may be a functionally active variant of the full-length mature sequence found in wild-type yeast comprising one or more genetic modifications as described herein, such as point mutations, but retaining enzymatic activity. Specifically, the functionally active variant of a native mature FCb2 peptide sequence as described herein comprises at least a cytochrome b2 domain and a flavin domain.

According to the invention, the recombinant FCb2 is different from amino acid sequence SEQ ID NO: 145 which is disclosed in WO 2021/167011 A1. According to a specific embodiment, the recombinant FCb2 is different from amino acid sequences which have been disclosed in WO 2021/167011 A1. According to a specific embodiment, the recombinant FCb2 is different from amino acid sequences given in SEQ ID NOs: 145 to 156 which have been disclosed in WO 2021/167011 A1. According to a specific embodiment, the recombinant FCb2 is different from amino acid sequence given in SEQ ID NOs: 145, 146, 147, 148, 149, 151, 152, 153, 154, 155 and/or 156 which have been disclosed in WO 2021/167011 A1. According to a specific embodiment, the recombinant FCb2 is different from a fragment or truncated form of said amino acid sequences given in SEQ ID NOs: 145 to 156 which have been disclosed in WO 2021/167011 A1. According to a specific embodiment, SEQ ID NO: 145 is the amino acid sequence of Saccharomyces cerevisiae FCb2 disclosed in WO 2021/167011 A1.

According to the invention, in the case an electrode comprises a recombinant FCb2, this FCb2 is different from amino acid sequences SEQ ID NOs: 146 to 156 which are disclosed in WO 2021/167011 A1. According to a specific embodiment, in the case the electrode comprises a recombinant FCb2, this FCb2 is different from the amino acid sequences given in SEQ ID NOs: 146, 147, 148, 149, 150, 151, 152, 153, 154, 155 and/or 156. According to a specific embodiment, in the case the electrode comprises a recombinant FCb2, this FCb2 is different from a fragment or truncated form of said amino acid sequences given in SEQ ID NOs: 146 to 156 which are disclosed in WO 2021/167011 A1.

According to one embodiment, the electrode comprising a recombinant FCb2 as described herein may be used in a biosensor for the determination of lactate or hydroxy acids in a sample.

According to one embodiment, a biosensor may be provided comprising the electrode, wherein said electrode comprises the recombinant FCb2 as described herein. Said biosensor may be used for analyte determination of lactate or hydroxy acids.

The flavin domain of FCb2, also termed dehydrogenase domain, is typically characterized by its enantiomer selectivity and specificity towards the natural lactate substrate which is commonly characterized by a KM in the low- or sub-millimolar range. Upon oxidation of lactate by a catalytic histidine base in the active site working in conjunction with the FMN cofactor, two electrons are subtracted, and pyruvate released as a reaction product. Subsequently, the flavin domain governs the transport of the so gained electrons towards a suitable electron acceptor like DCIP or the partnering cytochrome domain whilst commonly being irresponsive to accepting dioxygen as an electron acceptor.

The cytochrome domain of FCb2, also termed heme domain or haem domain, is typically characterized by its ability to shuttle electron from the neighboring flavin domain to external electron acceptors like cytochrome c, as is the physiological case, or artificial electrodes. Typically, the contained heme cofactor of the “b” type is coordinated by a brace of ligating histidines on opposing sides of the heme.

In a specific embodiment, the recombinant FCb2 described herein comprises the flavin domain of S. cerevisiae FCb2, comprising or consisting of residues 197-563 of SEQ ID NO:3, and the cytochrome domain of S. cerevisiae FCb2, comprising or consisting of residues 88-165 of SEQ ID NO:3, or it comprises the respective homologues of these domains of a different yeast.

For example, in S. cerevisiae the mature FCb2 sequence starts immediately after the I/L/V-x-N/A/L motif, and starts with the amino acids E P K L D (SEQ ID NO:45).

In a specific example, the recombinant FCb2 described herein comprises a sequence based on the FCb2 of S. cerevisiae and comprises a truncated signal peptide sequence as described herein, preferably comprising any one of SEQ ID NO:37, 38 or 39, and comprises a mature FCb2 peptide sequence which comprises:
a cytochrome-b2 domain spanning amino acid residues 88-165 of SEQ ID NO:3;
optionally a hinge region spanning residues 177-190 of SEQ ID NO:3, connecting the cytochrome b2 domain and the flavin domain,
a flavin domain spanning residues 197-563 of SEQ ID NO:3; and
optionally a tail region comprising residues 567-591 of SEQ ID NO3.

In a specific example, the mature FCb2 sequence of the recombinant FCb2 comprises or consists of SEQ ID NO:2, or a functionally active variant thereof comprising one or more genetic modifications as described herein, such as e.g. point mutations or deletions, and comprising at least the cytochrome b2 domain and the flavin domain of the FCb2 of S. cerevisiae.

The term “activity” as used herein e.g., in the context of an enzyme activity, shall refer to a functionally active molecule. A functional enzyme is specifically characterized by a catalytic center recognizing the enzyme substrate and catalysing the conversion of the substrate to a conversion product. For flavocytochrome b2 the preferred substrate is L-lactate and the conversion product is pyruvate. Enzyme variants are considered functional upon determining their enzymatic activity in a standard test system, e.g. wherein the enzymatic activity is at least 50% of the activity of the parent (not modified or wild-type enzyme), or at least any of 60%, 70%, 80%, 90%, 100%, or even more than 100%.

The enzymatic activity of a flavocytochrome b2 variant can be readily determined by assays known in the art, such as assays determining the colorimetric reduction of cytochrome c, ferricyanide or 2,6-dichloroindophenol (DCIP). Specifically, the recombinant FCb2 described herein comprises an enzymatic activity of at least 1 U/mg as determined by the cytochrome c assay described herein and as described by Disp Lê et al. (Diêp Lê et al. 2009).

In general, cytochrome c takes electrons only from heme b2, while ferricyanide is reduced both by the heme and by the flavin semiquinone in the enzyme. DCIP does not take electrons from the heme b but only from the flavin.

The enzymatic activity of a flavocytochrome b2 variant can be determined by a cytochrome c assay assessing the enzymatic activity from the colorimetric reduction of cytochrome c (CytC) at 30° C. and 550 nm, e.g. as previously described by Diêp Lê et al. (Diêp Lê et al. 2009); molar extinction coefficient=20 mM−1 cm−1. The assay mixture is buffered at pH 7.4 with 11 mM potassium phosphate, 137 mM NaCl, 3 mM KCl and contains 10 mM lactate 20 μM CytC, which acts as a terminal electron acceptor and specifically detects the activity of the whole enzyme as a product of all partial electron transfers (flavin and haem domain). The CytC assay thereby provides a measure of the efficiency of the intramolecular electron transfer (IET) between both domains and to external electron acceptors as an indication of the enzyme's response on electrodes. One unit of enzymatic activity is defined as the amount of enzyme that oxidizes 1 μmol of lactate per min under the assay conditions (U/mg). The reaction stoichiometry of lactate:CytC is 1:2, since two electrons are gained per lactate molecule and transferred individually to 2 molecules CytC. For the detection of activity with other substrates, lactate can be exchanged for other compounds.

The enzymatic activity of flavocytochrome b2 variants can also be determined by assessing the colorimetric reduction of ferricyanide (Fe(III)CNs₆³⁻) at 30° C. and 420 nm, e.g. as was described previously (Diêp Lê et al. 2009); molar extinction coefficient=1.0 mM−1 cm−1. The assay mixture is formulated as is described for the CytC assay but contains 13 mM ferricyanide instead of CytC. The reaction stoichiometry of lactate:ferricyanide is 1:2, since two electrons are gained per lactate molecule and transferred individually to 2 molecules ferricyanide. The reduction of ferricyanide is not specific for the terminal electron transfer by the heme as it can occur at the FMN domain similarly. For the detection of activity with other substrates, lactate can be exchanged for other compounds.

The enzymatic activity of flavocytochrome b2 variants can also be determined by assessing the colorimetric reduction of 2,6-dichloroindophenol (DCIP) at 30° C. and 600 nm, e.g. as described previously in (Gaume et al. 1995); molar extinction coefficient=22 mM−1 cm−1. The assay mixture is formulated as is described for the CytC assay but contains 0.1 mM DCIP instead of CytC. The reaction stoichiometry of lactate:DCIP is 1:1, since two electrons are gained per lactate molecule and transferred a single DCIP molecule. In contrast to CytC and ferricyanide, DCIP accept electrons from the primary electron transfer at the FMN domain, prior to shuttling to the partnering heme. For the detection of activity with other substrates, lactate can also be exchanged for other compounds.

The terms “increase in activity”, or “increased activity” or the like used herein may refer to a detectable increase in activity of an enzyme. The terms “increase in activity”, or “increased activity” used herein may mean that a modified enzyme, such as e.g. the FCb2 variants comprising truncated signal peptide sequences as described herein, shows higher activity than a comparable enzyme of the same type, like an enzyme that does not have the particular genetic modification. For example, activity of a modified or engineered enzyme may be higher than activity of a non-engineered enzyme of the same type, for example, a wild-type enzyme by about 5% or more, about 10% or more, about 15% or more, about 20% or more, about 30% or more, about 50% or more, about 60% or more, about 70% or more, or about 100% or more. The activity of a particular protein or enzyme in a recombinant or engineered cell may be higher than the activity of a protein or enzyme of the same type in a parent cell, for example, a non-engineered cell by about 5% or more, about 10% or more, about 15% or more, about 20% or more, about 30% or more, about 50% or more, about 60% or more, about 70% or more, or about 100% or more. Increased activity of an enzyme or protein in a cell may be verified by any methods known in the art.

The term “functional variant” or “functionally active variant” also includes naturally occurring allelic variants, as well as mutants or any other non-naturally occurring variants. As is known in the art, an allelic variant, or also referred to as homologue, is an alternate form of a nucleic acid or peptide that is characterized as having a substitution, deletion, or addition of one or more nucleotides or amino acids that does essentially not alter the biological function of the nucleic acid or polypeptide. Specifically, a functional variant may comprise a substitution, deletion and/or addition of 1, 2, 3, 4, 5, 6, 7, 8, 9, 10, 11, 12, 13, 14, 15, 16, 17, 18, 19 or 20 amino acid residues, or a combination thereof, specifically which substitutions, deletions and/or additions are conservative modifications and do not decrease the enzyme's specific activity. Specifically, a functionally active variant of FCb2 as described herein comprises specific enzymatic activity towards lactate of at least 1 U/mg, as determined by the CytC assay as described herein.

Specifically, a functional variant as described herein comprises no more than or up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 amino acid substitutions, deletions and/or additions, specifically which are conservative modifications and do not decrease the enzyme's specific activity. Specifically, a functionally active variant as described herein comprises up to 15, preferably up to 10 or 5, amino acid substitutions, deletions and/or additions, specifically which are conservative modifications and do not decrease the enzyme's specific activity.

Specifically, a functionally active variant described herein comprises at least 40, 50, 60, 70, 80 or 90% or even more of the enzymatic activity of the respective wild type enzyme. According to a specific example, if the recombinant FCb2 described herein is derived from the S. cerevisiae FCb2, said recombinant FCb2 is a functionally active variant if it comprises at least 40, 50, 60, 70, 80 or 90% or even more of the enzymatic activity of the FCb2 comprising SEQ ID NO:3.

Functional variants may be obtained by sequence alterations in the polypeptide or the nucleotide sequence, e.g. by one or more point mutations, wherein the sequence alterations retain or improve a feature of the enzyme, such as its stability for example. Such sequence alterations can include, but are not limited to, (conservative) substitutions, additions, deletions, mutations and insertions. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc.

A point mutation is particularly understood as the engineering of a polynucleotide that results in the expression of an amino acid sequence that differs from the non-engineered amino acid sequence in the substitution or exchange, deletion or insertion of one or more single (non-consecutive) or doublets of amino acids for different amino acids.

According to a specific embodiment, the FCb2 described herein comprises one or more tag sequences, specifically N-terminal tag sequences. Specifically, such tag sequence is C-terminal of the N-terminal methionine of the recombinant FCb2 described herein. Such tag sequence may comprise any number of amino acids of more than 2, 4, 5, 6 or 10 amino acids and up to 20 or 50 or more amino acids. Specifically, tag sequences used herein may be any tag sequence known to the person skilled in the art. Specifically, tag sequences used herein are selected from affinity tags, solubility enhancement tags or monitoring tags.

Affinity tags are amino acid sequences that can be used for example for the purification of proteins where they are attached to. These affinity tags have high affinity to appropriate ligands of a solid support, like chromatography resins or directly to the resins. By selectively binding of the protein having the affinity tag to the particular resin the protein can be purified highly effective by one chromatography step only. According to a specific embodiment, affinity tag sequences used herein are selected from histidine (His) tag, specifically a poly-histidine tag, poly-arginine tag, FLAG tag, Strep tag, streptavidin-binding peptide (SBP) tag, calmodulin-binding peptide (CBP) tag, S-tag, HA tag, c-Myc tag, and SUMO tag, or any other tag known to be useful for the efficient purification of a protein it is fused to. Preferably, the tag is a His tag comprising one or more H, specifically a hexahistidine tag. Specifically, proteins comprising a poly-, or hexa-histidine tag (His-tag) can be captured and purified using chromatography, e.g. by Immobilized Metal Affinity Chromatography (IMAC).

Solubility enhancement tags can be fused N-terminal to the FCb2 described herein. Solubility enhancement tags can increase the titer of the soluble protein when expressed in a host cell, e.g. in the cytosol of P. pastoris, compared to expression of the proteins without the tag. According to a further specific embodiment, solubility enhancement tag sequences used herein are selected from calmodulin-binding peptide (CBP), poly Arg, poly Lys, protein D tag (dTAG), Z domain of Staphylococcal protein A, and thioredoxin or any other tag known to improve the solubility of the protein it is fused to e.g. during expression in a host cell. Specifically, the solubility enhancement tag is a T7 tag, preferably selected from the group consisting of T7A, T7A1, T7A2, T7A3, T7A4, T7A5, T7B, T7B1, T7B2, T7B3, T7B3, T7B4, T7B5, T7B6, T7B6, T7B7, T7B8, T7B9, T7B10, T7B11, T7B12, T7B13, and T7C.

According to a further specific embodiment, the monitoring tag sequence used herein is m-Cherry, GFP or f-Actin or any other tag useful for detection or quantification of the recombinant enzyme during production steps including fermentation, isolation and purification by simple in-situ, inline, online or atline detectors, like UV, IR, Raman, fluorescence and the like.

Further provided herein are methods of producing the recombinant FCb2 described herein in a host cell.

As an FCb2 molecule requires the cofactors FMN and heme b incorporated in their 3-D structure for the establishment of direct electron transfer from the substrate lactate, over FMN, heme b, and finally to an electrode, the expression of the recombinant FCb2 of the invention has to be performed using a host capable of producing sufficient amounts of these cofactors. Especially for the sufficient production of the heme b cofactor, a yeast organism is used according to the invention. Yeast cells are able to successfully produce recombinant proteins having heme b as cofactor. In contrast, bacterial cells generally do not allow to produce sufficient amounts of the iron-cofactor heme b. Also, the expression system has an impact on the specific activity and the stability of an enzyme. The term “host cell” as referred to herein is understood as any yeast cell type that is susceptible to transformation, transfection, transduction, or the like with nucleic acid constructs or expression vectors comprising polynucleotides encoding expression products described herein, or susceptible to otherwise introduce the recombinant enzyme described herein. Specifically, the host yeast cells are maintained under conditions allowing expression of the enzyme. Host yeast cells can be haploid, diploid or polyploid cells.

Also described herein is also a “host cell line” or “production cell line”, which is commonly understood to be a yeast cell line ready-to-use for cultivation/culturing in a bioreactor to obtain the product of a production process, such as the recombinant enzymes described herein. The yeast host or yeast cell line as described herein is particularly understood as a recombinant yeast organism, which may be cultivated/cultured to produce the desired recombinant protein.

Genetic modifications of such host cells include modifications that introduce a polynucleotide encoding a polypeptide into a cell; a modification that substitutes, adds (i.e., inserts), or deletes one or more nucleotides of the genetic material of a parent cell, including a chemical modification (exposure to a chemical) resulting in a change to the genetic material of a parent cell. Genetic modification includes a heterologous or homologous modification of referenced species. Genetic modification further includes a modification of a coding region for polypeptides. Genetic modification also includes a modification of non-coding regulatory regions that changes expression of a gene or function of an operon. Non-coding regions include 5-non-coding sequence (5′ of a coding sequence) and 3-non-coding sequence (3′ of a coding sequence).

The “gene” used herein refers to a nucleic acid fragment that encodes a particular protein such as the recombinant FCb2 described herein, which may optionally include at least one regulatory sequence, such as a 5-non-coding sequence and a 3-non-coding sequence (3′ and 5′ in reference to the position relative to the coding sequence).

In some embodiments, the gene encoding the recombinant FCb2 described herein may be introduced into a yeast cell and inserted into an endogenous genetic material (for example, chromosome) of a yeast cell. This gene may be inserted into one or more locations of a particular endogenous gene of the yeast cell to disrupt the endogenous gene. The particular endogenous genes to be disrupted may include the native FCb2 gene.

The gene that encodes the recombinant FCb2 may be introduced in the yeast cell, but not inserted into the endogenous genetic material of the yeast cell. For example, the gene may be included in an expression vector, such as a plasmid, and remain separated from the endogenous genetic material of the yeast cell.

The gene may be introduced in an expressionable form into a yeast cell, and expressed to generate a gene product thereof in the yeast cell. The expressionable form may be a structure in which the gene is operably linked to an expression regulatory sequence. For example, the gene may be operably linked to at least one sequence selected from an exogenous enhancer, an operator, a promoter, and a transcription terminator to be expressionable in a yeast cell, or may be linked to an endogenous regulatory sequence of the yeast cell to be expressionable in the yeast cell.

The gene may be introduced into the yeast host cell using any known method of introducing a genetic material into a yeast cell (see e.g. R. Gietz et al., Biotechniques 30:816-831, April 2001). The introducing may include a spheroplast method, intact yeast cell transformation, electroporation, or a combination thereof. For example, intact yeast cell transformation may use a particular monovalent alkali cation (Na+, K+, Rb+, Cs+, and Li+) in combination with PEG to promote uptake of DNA, such as a plasmid, by a yeast cell. For example, intact yeast cell transformation may include applying a heat shock to an aqueous solution of PEG, LiAc, carrier ssDNA, plasmid DNA, and a yeast cell. For example, electroporation may include applying an electric pulse to a mixed medium including a yeast cell and DNA, such as plasmid DNA.

In the introduction into the yeast, the gene may be included in a vector, with a homologous sequence with respect to an endogenous genetic material of a parent cell of a yeast cell. The homologous sequence is complementary to a target sequence present in an endogenous genetic material of a parent yeast cell, and accordingly may be substituted with the target sequence by homologous recombination. The vector may include two sequences which are respectively homologous to the 5′ end and 3′ end of the target sequence. In this regard, the introducing may include culturing the yeast cell under a selection pressure during or after the contacting. The selection pressure may indicate a material or state that enables to select only cells where homologous recombination has occurred. The selection pressure may include culturing in the presence of antibiotics. In this regard, the vector may include a gene that provides antibiotic resistance to the yeast cell, for example, a gene that encodes an enzyme that decomposes antibiotics.

The term “cell culture” or “cultivation” (“culturing” is herein synonymously used), also termed “fermentation”, with respect to a host cell line is meant to be the maintenance of cells in an artificial, e.g., an in vitro environment, under conditions favoring growth, differentiation or continued viability, in an active or quiescent state, of the cells, specifically in a controlled bioreactor according to methods known in the industry. When cultivating, a cell culture is brought into contact with the cell culture media in a culture vessel or with substrate under conditions suitable to support cultivation of the cell culture. In certain embodiments, a culture medium as described herein is used to culture cells according to standard cell culture techniques that are well-known in the art for cultivating or growing yeast cells.

Cultivation of the yeast host cells may be in one, two or multiple phases.

According to a specific embodiment, cell growth and production of the recombinant protein is in a single phase. In this case, the medium used in the cultivation process comprises the respective components required for the production of the protein from the beginning of the cultivation process.

Cell culture media provide the nutrients necessary to maintain and grow cells in a controlled, artificial and in vitro environment. Characteristics and compositions of the cell culture media vary depending on the particular cellular requirements. Important parameters include osmolality, pH, and nutrient formulations. Feeding of nutrients may be done in a continuous or discontinuous mode according to methods known in the art.

Cell culture may be a batch process or a fed-batch process. A batch process is a cultivation mode in which all the nutrients necessary for cultivation of the cells, and optionally including the substrates necessary for production of the FCb2 as described herein, are contained in the initial culture medium, without additional supply of further nutrients during fermentation. In a fed-batch process, a feeding phase takes place after the batch phase. In the feeding phase one or more nutrients, such as an inducer, e.g. methanol, are supplied to the culture by feeding. In certain embodiments, the method described herein is a fed-batch process. Specifically, a host cell transformed with a nucleic acid construct encoding the enzyme as described herein, is cultured in a growth phase medium and transitioned to an induction phase medium in order to produce the recombinant proteins described herein.

In another embodiment, host cells described herein are cultivated in continuous mode, e.g. a chemostat. A continuous fermentation process is characterized by a defined, constant and continuous rate of feeding of fresh culture medium into the bioreactor, whereby culture broth is at the same time removed from the bioreactor at the same defined, constant and continuous removal rate. By keeping culture medium, feeding rate and removal rate at the same constant level, the cultivation parameters and conditions in the bioreactor remain constant.

Expression products such as polypeptides, proteins or enzymes as described herein may be introduced into a host cell either by introducing the respective coding polynucleotide or nucleotide sequence for expressing the expression products within the host cell, or by introducing the respective expression products which are within an expression system or isolated. Any of the known procedures for introducing expression cassettes, vectors or otherwise introducing (e.g., coding) nucleotide sequences into host cells may be used (see, e.g., Sambrook et al.).

The invention specifically allows for the production process to be performed on a pilot or industrial scale.

A growth medium allowing the accumulation of biomass as described herein, specifically a basal growth medium, typically a carbon source, a nitrogen source, a source for sulphur and a source for phosphate. Typically, such a medium comprises furthermore trace elements and vitamins, and may further comprise amino acids, peptone or yeast extract.

The fermentation preferably is carried out at a pH ranging from 3 to 7.5.

Typical fermentation times are about 24 to 120 hours with temperatures in the range of 20° C. to 35° C., preferably 22-30° C.

Specifically, the cells are cultivated under conditions suitable to produce the recombinant enzymes described herein, which can be purified from the cells or culture medium.

The term “exogenous” as used herein refers to a referenced molecule (e.g., nucleic acid) that has been introduced into a host cell. A nucleic acid may be exogenously introduced into a host in any suitable manner. For example, a nucleic acid can be introduced into a host cell and inserted into a host chromosome, or the nucleic acid can be introduced into the host as non-chromosomal genetic material, such as an expression vector (e.g., a plasmid) that does not integrate into the host chromosome. A nucleic acid encoding a protein should be introduced in an expressionable form (i.e., so that the nucleic acid can be transcribed and translated).

The term “endogenous” refers to a referenced molecule (e.g., nucleic acid) already present in the host cell prior to a particular genetic modification (e.g., a genetic composition, trait, or biosynthetic activity of a “wild-type” cell or a parent cell).

The term “heterologous” refers to a referenced molecule (e.g., nucleic acid) derived from a source other than referenced species; and the term “homologous” refers to a molecule (e.g., nucleic acid) or activity derived from a host parent cell. Accordingly, an exogenous molecule (e.g., expression of an exogenous coding nucleic acid) may be heterologous (e.g., a coding nucleic acid from a different species) or homologous (e.g., an additional copy of a coding nucleic acid from the same species).

The term “expression” as used herein regarding expressing a polynucleotide or nucleotide sequence, is meant to encompass at least one step selected from the group consisting of DNA transcription into mRNA, mRNA processing, non-coding mRNA maturation, mRNA export, translation, protein folding and/or protein transport. Nucleic acid molecules containing a desired nucleotide sequence may be used for producing an expression product encoded by such nucleotide sequence e.g., proteins or transcription products such as RNA molecules. To express a desired nucleotide sequence, an expression system is conveniently used, which can be an in vitro or in vivo expression system, as necessary to express a certain nucleotide sequence by a host cell or host cell line. Typically, host cells are transfected or transformed with an expression system comprising an expression cassette that comprises the desired nucleotide sequence and a promoter operably linked thereto optionally together with further expression control sequences or other regulatory sequences. Specific expression systems employ expression constructs such as vectors comprising one or more expression cassettes.

The term “expression construct” as used herein, means the vehicle, e.g. vectors or plasmids, by which a DNA sequence is introduced into a host cell so as to transform the host and promote expression (e.g. transcription and translation) of the introduced sequence. “Expression construct” as used herein includes both, autonomously replicating nucleotide sequences as well as genome integrating nucleotide sequences.

The terms “vector”, “DNA vector” and “expression vector” mean the vehicle by which a DNA sequence (e.g. a foreign gene) can be introduced into a host cell, so as to transform the host and promote expression (e.g. transcription and translation) of the introduced sequence. Vector” as used herein includes both, autonomously replicating nucleotide sequences as well as genome integrating nucleotide sequences, such as artificial chromosomes. Plasmids are preferred vectors of the invention.

In specific embodiments, an expression vector may contain more than one expression cassettes, each comprising at least one coding sequence and a promoter in operable linkage.

A “cassette” refers to a DNA coding sequence or segment of DNA that codes for an expression product that can be inserted into a vector at defined restriction sites. The cassette restriction sites are designed to ensure insertion of the cassette in the proper reading frame. An “expression cassette” as used herein refers to nucleic acid molecules containing a desired coding sequence and control sequences in operable linkage, so that an expression system can use such expression cassette to produce the respective expression products, including e.g., encoded proteins or other expression products. Certain expression systems employ host cells or host cell lines which are transformed or transfected with an expression cassette, which host cells are then capable of producing expression products in vivo. In order to effect transformation of host cells, an expression cassette may be conveniently included in a vector, which is introduced into a host cell; however, the relevant DNA may also be integrated into a host chromosome. A coding sequence is typically a coding DNA or coding DNA sequence which encodes a particular amino acid sequence of a particular polypeptide or protein, or which encodes any other expression product.

The term “expression vector” means a linear or circular DNA molecule that comprises a polynucleotide encoding a polypeptide and is operably linked to control sequences that provide for its expression. Vectors typically comprise DNA sequences that are required for the transcription of cloned recombinant nucleotide sequences, i.e. of recombinant genes and the translation of their mRNA in a suitable host organism. A coding DNA sequence or segment of DNA molecule coding for an expression product can be conveniently inserted into a vector at defined restriction sites. To produce a vector, heterologous foreign DNA can be inserted at one or more restriction sites of a vector DNA, and then is carried by the vector into a host cell along with the transmissible vector DNA. It is preferred that a vector comprises an expression system, e.g. one or more expression cassettes. Expression cassette restriction sites are designed to ensure insertion of the cassette in the proper reading frame.

To obtain expression, a sequence encoding a desired expression product, such as e.g. any of the polypeptides or proteins described herein, is typically cloned into an expression vector that contains a promoter to direct transcription. Appropriate expression vectors typically comprise regulatory sequences suitable for expressing coding DNA. Examples of regulatory sequences include promoter, operators, enhancers, ribosomal binding sites, and sequences that control transcription and translation initiation and termination. The regulatory sequences are typically operably linked to the DNA sequence to be expressed.

A promoter is herein understood as a DNA sequence which initiates, regulates, or otherwise mediates or controls the expression of the coding DNA. Promoter DNA and coding DNA may be from the same gene or from different genes, and may be from the same or different organisms. Recombinant cloning vectors often include one or more replication systems for cloning or expression, one or more markers for selection in the host, e.g., antibiotic resistance, one or more nuclear localization signals (NLS) and one or more expression cassettes.

The term “sequence identity” as used herein is understood as the relatedness between two amino acid sequences or between two nucleotide sequences and described by the degree of sequence identity or sequence complementarity. The sequence identity of a variant, homologue or orthologue as compared to a parent nucleotide or amino acid sequence indicates the degree of identity of two or more sequences. Two or more amino acid sequences may have the same or conserved amino acid residues at a corresponding position, to a certain degree, up to 100%. Two or more nucleotide sequences may have the same or conserved base pairs at a corresponding position, to a certain degree, up to 100%.

Sequence similarity searching is an effective and reliable strategy for identifying homologs with excess (e.g., at least 50%) sequence identity. Sequence similarity search tools frequently used are e.g., BLAST, FASTA, and HMMER.

Sequence similarity searches can identify such homologous proteins or polynucleotides by detecting excess similarity, and statistically significant similarity that reflects common ancestry. Homologues may encompass orthologues, which are herein understood as the same protein in different organisms, e.g., variants of such protein in different organisms or species.

To determine the % complementarity of two complementary sequences, one of the two sequences needs to be converted to its complementary sequence before the % complementarity can then be calculated as the % identity between the first sequence and the second converted sequences using the above-mentioned algorithm.

“Percent (%) identity” with respect to an amino acid sequence, homologs and orthologues described herein is defined as the percentage of amino acid residues in a candidate sequence that are identical with the amino acid residues in the specific polypeptide sequence, after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared. In case of percentages determined for sequence identities, it is possible that arithmetical decimal places may result which are not possible with regard to full nucleotides or amino acids. In this case, the percentages shall be rounded up to whole nucleotides or amino acids.

For purposes described herein, the sequence identity between two amino acid sequences is determined using standard methods, e.g. using the NCBI BLAST program version 2.2.29 (Jan. 6, 2014).

“Percent (%) identity” with respect to a nucleotide sequence e.g., of a nucleic acid molecule or a part thereof, in particular a coding DNA sequence, is defined as the percentage of nucleotides in a candidate DNA sequence that is identical with the nucleotides in the DNA sequence, after aligning the sequence and introducing gaps, if necessary, to achieve the maximum percent sequence identity, and not considering any conservative substitutions as part of the sequence identity. Alignment for purposes of determining percent nucleotide sequence identity can be achieved in various ways that are within the skill in the art, for instance, using publicly available computer software. Those skilled in the art can determine appropriate parameters for measuring alignment, including any algorithms needed to achieve maximal alignment over the full length of the sequences being compared.

Optimal alignment may be determined with the use of any suitable algorithm for aligning sequences, non-limiting examples of which include the Smith-Waterman algorithm, the Needleman-Wunsch algorithm, MAFFT based algorithms: multiple alignment using fast fourier transform, algorithms based on the Burrows-Wheeler Transform (e.g., the Burrows Wheeler Aligner), ClustalW, Clustal X, BLAT, Novoalign (Novocraft Technologies; available at novocraft.com), ELAND (Illumina, San Diego, CA), SOAP (available at soap.genomies.org.cn), and Maq (available at maq.sourceforge.net).

The examples described herein are illustrative of the present invention and are not intended to be limitations thereon. Many modifications and variations may be made to the techniques described and illustrated herein without departing from scope of the invention. Accordingly, it should be understood that the examples are illustrative only and are not limiting upon the scope of the invention.

### EXAMPLES

Flavocytochrome b2 (FCb2) has two functional domains that are connected via a “hinge” linker (57 kDa monomer). The FCb2 from S. cerevisiae is the best studied representative and has been crystallized (PDB: 1 FCB). The following sections have been identified:
the N-terminal amino acid residues, up to amino acid residue 88, are assumed to be responsible for cellular targeting into the mitochondria. This so-called “translocation peptide” is cleaved upon translocation;
Cytochrome-b2 domain: <10 kDa domain with non-covalent heme b: (spanning amino acid residues 88-165);
Hinge region with domain-connecting linker (spanning residues 177-190), connecting the cytochrome b2 domain and the flavin domain;
Flavin domain of approx. 45 kDa, containing an FMN (spanning residues 197-563);
Tail region (comprising residues 567-591), extends at the C-terminus and wraps around dimers.

The physiological role of FCb2 in yeast relies on its presence in the mitochondrial transmembrane space (IMS) and its occurrence in this mitochondrial sub-compartment has been confirmed by two publications on the FCb2 from S. cerevisiae (Grandier-Vazeille et al. 2001; Vögtle et al. 2012). As the terminal destination of the FCb2 within the yeast cell could be experimentally confirmed, its synthesis as a precursor in the cytosol is very likely and relies on the presence of a targeting signal within the precursor to facilitate this directed transport from one compartment to the other.

Analysis of FCb2 sequences revealed that FCb2 genes indeed contain N-terminal sequence parts that are not contained in the mature FCb2 enzyme and putatively removed as targeting signals or signal peptides during incorporation, as biochemical data underlined for the FCb2 from S. cerevisiae (Ghrir, Becam, and Lederer 1984; Lederer et al. 1985) the only FCb2 for which a crystal structure was resolved so far PDB: 1 FCB (Xia and Mathews 1990). Additional to ScFCb2, literature provides data for the FCb2 from Wickerhamomyces anomalus (WaFCb2, formerly Hansenula anomala), where a mitochondrial translocation peptide cleavage is indicated, as literature reports processing of the N-terminus leading to the mature WaFCb2 (Haumont et al. 1987; Black et al. 1989).

It is believed that rather than sharing a consensus sequence, the import into the mitochondria relies on the occurrence of sequence stretches with similar physicochemical properties (Abe et al. 2000). Analysis of peptidase cleavage sites of the S. cerevisiae mitochondrial proteome identified several sequence patterns in the pre-sequence, dependent on the route through the mitochondrial membranes. Still, conservation of these patterns was below 65% highlighting that sequence conservation was inherently inhomogeneous (Gakh, Cavadini, and Isaya 2002).

The transport of FCb2 into the IMS needs to at least involve transport to the mitochondria and two subsequent steps: (1) transfer through the outer mitochondrial membrane prior to (2) incorporation into the intermembrane space. Although the fundamental processes that are involved in step (2), the ultimate targeting of FCb2 enzymes to the IMS are still enigmatic, a more comprehensive understanding of the preceding step (1) of trafficking through the outer mitochondrial membrane (and subsequently inner membrane) is available. This initial step is governed by signal recognition by receptors and membrane integrated translocases and ensuing release via cleavage by specialized mitochondrial processing peptidases (MPP). Publications describing sequence analyses of mitochondrial precursor proteins revealed three main features found in the majority of pre-sequences cleaved by MPP (Gakh, Cavadini, and Isaya 2002), namely,
(i) an overall positive charge,
(ii) a predicted ability to form an amphiphilic α-helix, and
(iii) the presence of an arginine residue at the −2 position (the “R-2 rule”).

Four cleavage site motifs are reported:

| | |
|---|---|
| xRx<>x(S/x) | (R-2 motif); |
| xRx(Y/x)<>(S/A/x)x | (R-3 motif) |
| xRx<>(F/L/I)xx(S/T/G)xxxx↓ | (R-10 motif, sequentially by MPP, MIP) |
| xx<>x(S/x) | (R-none motif) |

In a survey of 71 mitochondrial matrix-targeting pre-sequences from S. cerevisiae, R-2, R-3 or R-10 motifs were found in only ˜65% of the pre-sequences. Thus, there is a relatively high degree of amino acid sequence degeneracy at MPP cleavage sites, and this limits the level of confidence with which such sites can be predicted by statistical or other methods. These predictions are often complicated by the fact that following an initial cleavage by MPP, many pre-sequences are cleaved again by other peptidases at different locations, with different requirements for substrate recognition. This is likely to be the case for a secondary processing of the FCb2 N-terminus.

### Example 1: Characterization of the N-Terminal Leader Sequence of FCb2

Only few online-tools are currently available that allow predicting the cellular targeting to mitochondria with N-terminal signal peptides
Mitofates (http://mitf.cbrc.jp/MitoFates/cgi-bin/top.cgi)
Predotar (http://urgi.versailles.inra.fr/predotar/predotar.html)
TargetP (http://www.cbs.dtu.dk/services/TargetP/)

Most established, TargetP-2.0 (http://www.cbs.dtu.dk/services/TargetP/) can predict cellular fates for various organisms and compartments. Tests were conducted with established FCb2 sequences of Saccharomyces cerevisiae (ScFCb2), Kluyveromyces marxianus (KmFCb2), Wickerhamomyces anomalus (WaFCb2), and Candida glabrata (CangFCb2) (SEQ ID NOs: 3, 7, 11, 16) to gain insight into prediction.

### Analysis of FCb2 Leader Sequences

FCb2 sequences obtained from BLAST search on Uniprot and NCBI were assembled and restricted to entries from yeasts (Saccharomycetacae). A total of 37 sequences were gathered, the 1 FCB crystal structure (SEQ ID NO: 1) served as a positive control since it contains the mature and processed N′-terminus. The Fasta files were downloaded from the BLAST search and loaded into Jalview. Sequence duplicates were removed from the list. The most probable sequence alignment was calculated using the MAFFT algorithm with G-INS-I presets and the alignment was inspected for obvious errors. The alignment was then truncated to the initial 164 positions until the conserved “WVVI” of the structurally relevant part and colorized for conservation.

In a second step, these relevant regions were analysed for conservation of certain residues using the WebLogo online tool (https://weblogo.berkeley.edu/logo.cgi).

### Results

Saccharomyces cerevisiae (ScFCb2)

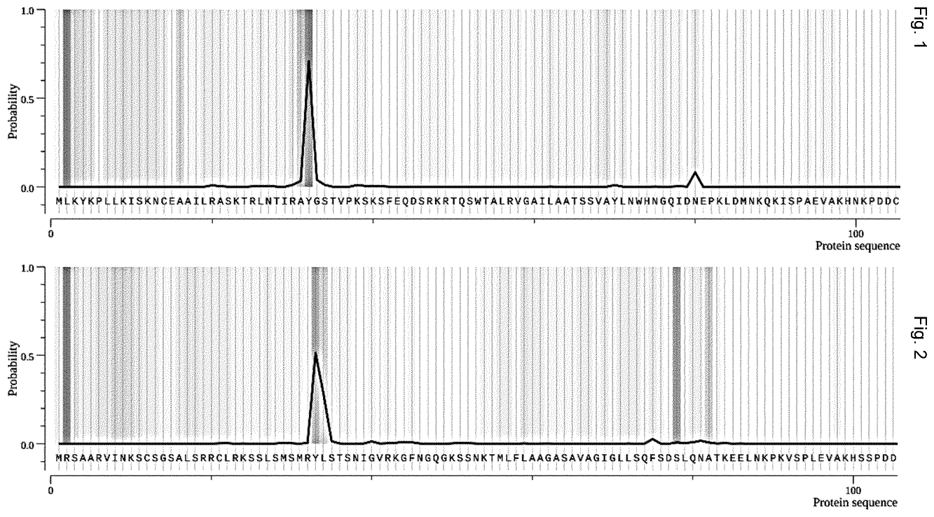

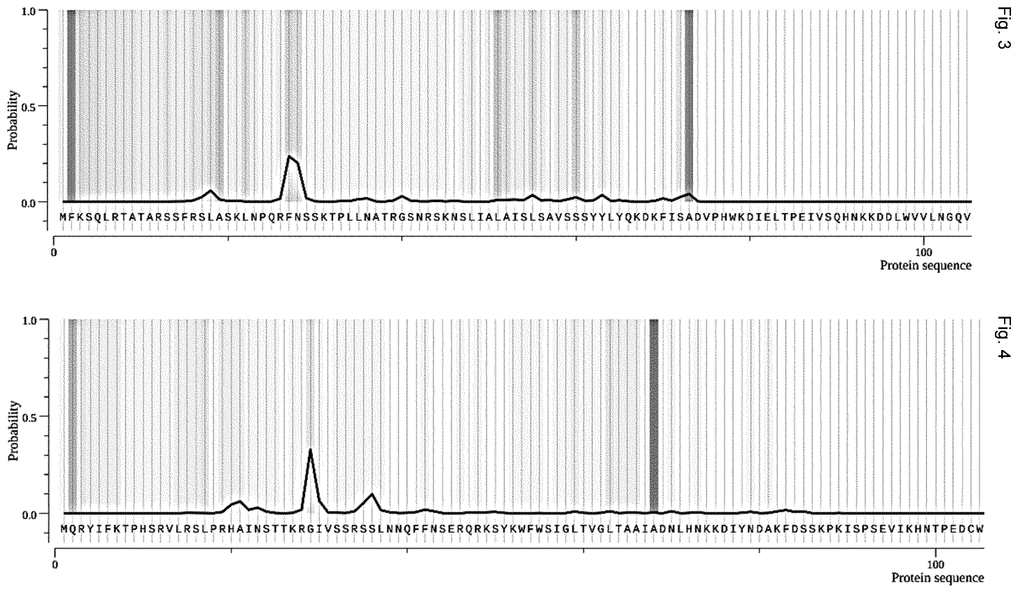

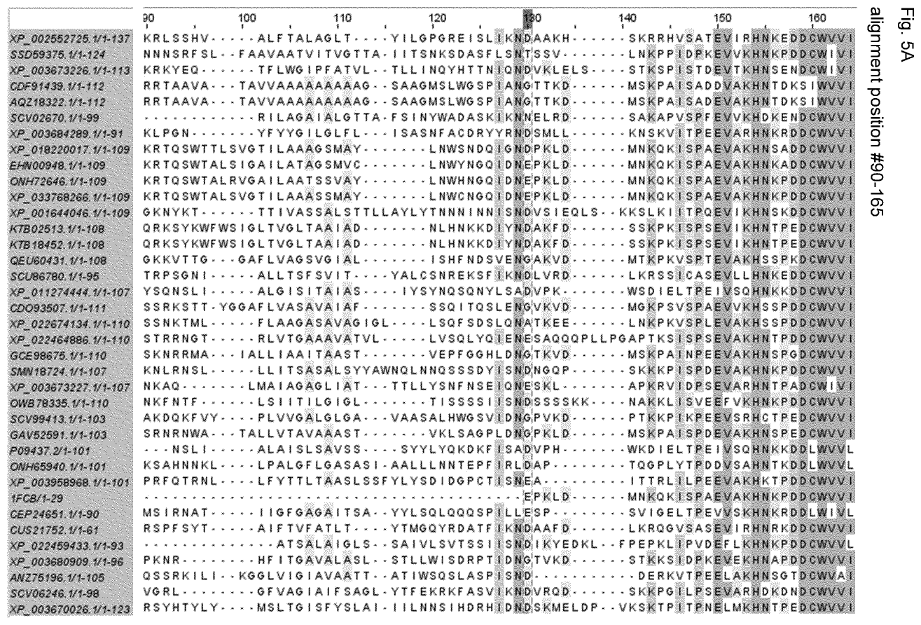

Target P (FIG. 1) recognized a mitochondrial import signal cleavage site with 93% confidence for position R-A-Y32< >G33, and reflects a TOM recognition and MPP cleavage R3 motif (R-x-Y/x< >S/A/x-) (< > represents a cut made at that position). Another peptidase cleavage site is projected at N80/81E. Cleavage at this position would result in the mature FCb2 protein sequence cleaving at: I-D-N< >E-P-K-L-D. . . .

Mitofates recognized the same MPP (mitochondrial processing peptidase) cleavage site with 99.6% confidence at the position RA-Y32< >G33. No additional peptidase cleavage sites were predicted by Mitofates.

Kluyveromyces marxianus (KmFCb2)

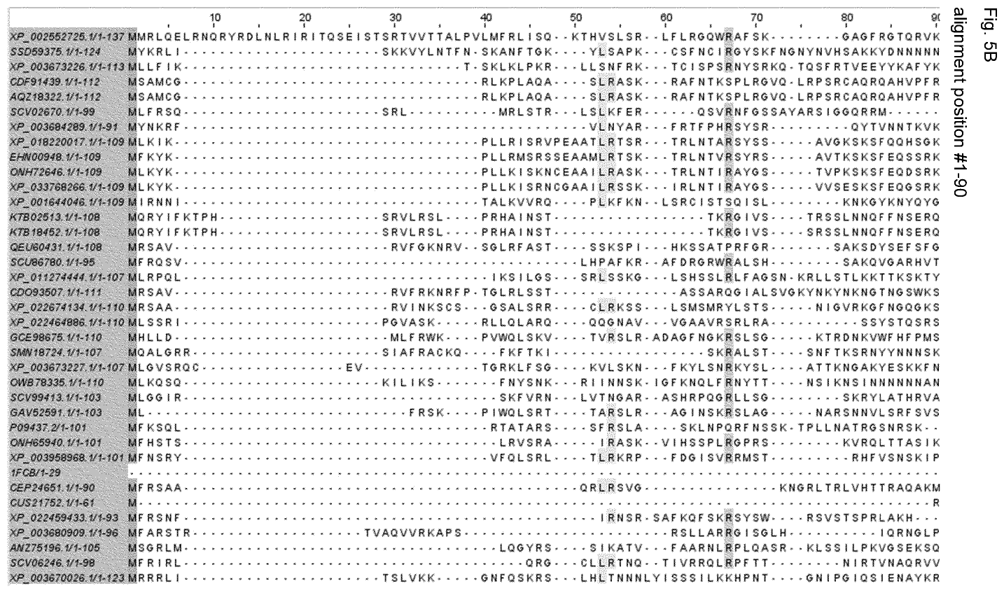

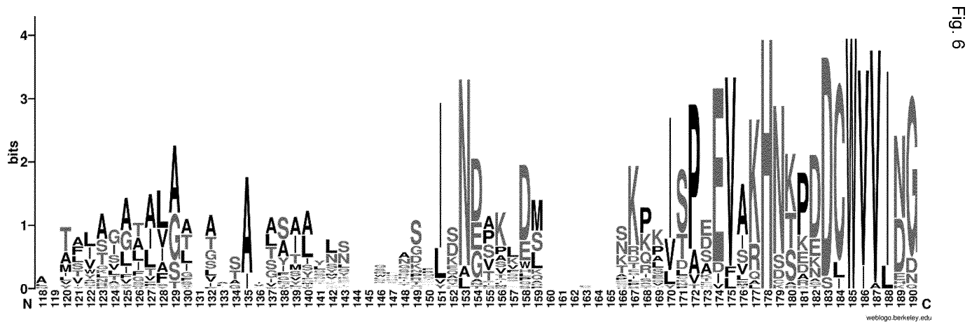

Target P (FIG. 2) recognized a mitochondrial import signal cleavage site with 92% confidence, this is for position R-Y33< >L34-S and reflects a TOM recognition and MPP cleavage R2 motif (R-x< >x-S). A second peptidase cleavage site is indicated but was not quantifiable.

Wickerhamomyces anomalus (WaFCb2)

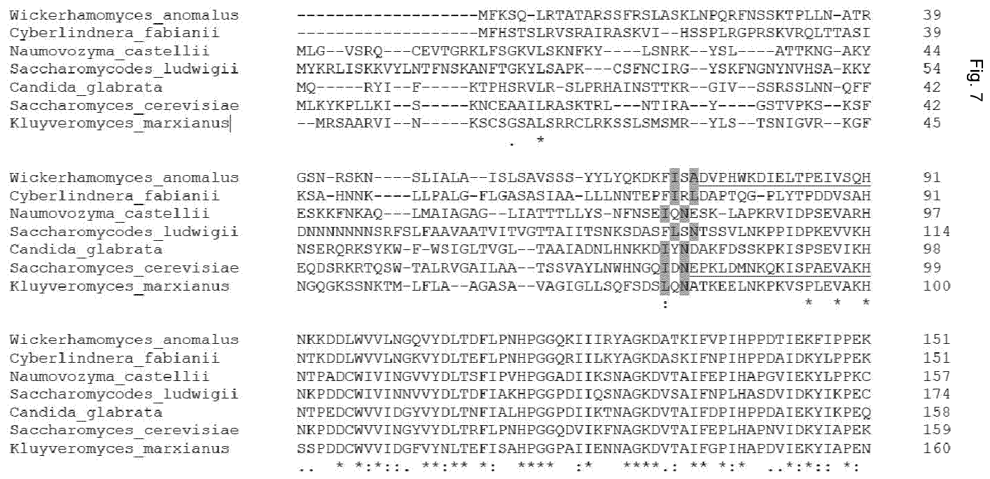

Target P (FIG. 3) recognized a mitochondrial import signal cleavage site with 92% confidence, this is for position Q-R-F27< >N28-S and reflects a TOM recognition and MPP cleavage R2 motif (R-x< >x-S). Other peptidase cleavage sites are indicated but were not quantifiable.

Candida glabrata (CancFCb2)

Target P (FIG. 4) recognized a mitochondrial import signal cleavage site with 78% confidence for position RG29< >130 and reflects a TOM recognition and MPP cleavage likely as R10 motif. Other peptidase cleavage sites are not found.

Generally, it was apparent that the available tools could detect the classical canonical TOM-dependent peptidase cleavage sites, which are necessary for step 1 of the mitochondrial import. In addition, there seemed to be indications for peptidase cleavage at the FCb2 relevant position as can be seen from the ScFCb2 analysis (FIG. 1). To confirm, a detailed sequence alignment of known FCb2 sequences was performed (FIG. 5A, 5B).

The conserved TOM-dependent MPP cleavage site was identified with good conservation at position 67 of the alignment which is highlighted in FIG. 5B (in FIG. 5, the position number indicates the alignment position rather than the respective residue positions), which confirms the results of the TargetP predictions: MPP R2 Motif (R-x< >x-S) and R3 Motif (R-x-Y/x< >S/A/x-)

Good agreement of sequences was also found further downstream (FIG. 5A), right before the conserved structural region of the FCb2 enzyme (FIG. 5A, position #143 and onwards). A potential (I/L/V-x-N/A/L) recognition site is reflected in ScFCb2 as IDN< >EPKLD. The I/L/V-x-N/A/L recognition site is located at #127-129, 25 positions upstream from the conserved His (alignment position #154) in ScFCb2.

There is a high degree of conservation within the #127-129 sequence stretch: the I/L/V-x-N/A/L motif is highlighted (FIG. 6). This stretch is expected to represent the so far unknown recognition sequence for secondary processing.

In the crystal structure of the ScFCb2, the matured N-terminus “EPLKD . . . ” fits well to this prediction. The structurally relevant parts of the mature enzyme are expected to be located further downstream. Still, activity and stability of the enzyme could be impacted by parts of the leader sequence, which was thus further tested herein.

As is observable from the alignment in FIG. 7, the N-terminus of the FCb2 sequences presents itself as quite inhomogeneous for the selected set of FCb2 enzymes. Still, it seems there is a pattern of around 5-10 residues upstream of the structurally important regions that could be responsible for recognition.

“V-x-x-H” is the first conserved part of the structural part and interacts with the heme upstream of that region, likely a short stretch of amino acids is responsible for recognition of the translocation peptide peptidase. This part is not present in ascomycotal sequences which are more distantly related to yeasts.

The maturated N-terminus of ScFCb2 starts with EPKLD (in PDB:1FCB) and similarities to closely related sequences are observable. There seems to be a recognition motif of hydrophobic amino acids (I, L, V), spacer and Asparagine, which was sometimes substituted in the alignment for A,L.

Subsequent to the putative recognition motif “I/L/V-x-N/A/L” charged amino acids (D, K, E) seem to be prominent but no clear homology could be identified (FIG. 6). Potentially, the maturated N-terminus is formed from the residues right thereafter and already has structurally relevant impact on the mature enzyme.

### Conclusion

The structural N-terminus of the mature FCb2 protein is generally well-conserved. The sequence of the N-terminus, however, is less well conserved, but several motifs were identified:
MPP motifs R2/R3 (TOM import), which were predicted from the literature, could be confirmed for the various FCb2 variants in this study; and
a conserved sequence prior to the structural start of the mature protein was identified and was termed I/L/V-x-N/A/L motif (also further referred to herein as “TransP recognition motif” or simply “TransP motif”).

Based on the identified I/L/V-x-N/A/L motif, several expression plasmids were constructed for the FCb2 variants of S. cerevisiae (ScFCb2), Wickerhamomyces anomalus (WaFCb2), Kluyveromyces marxianus (MaxFCb2), Candida glabrata (CangFCb2), Naumovozyma castelli (NacaFCb2) and Cyberlindera fabianii (CyberFCb2). The genetic sequences encoding the FCb2 variants were truncated to the putative I/L/V-x-N/A/L recognition motif at different lengths, and some variants were also equipped with an N-terminal His-tag.

### Example 2: Screening of FCb2 Variants Comprising Truncated Translocation Peptide Sequences

Activity screening of FCb2 variants was successful using the protocol published by Kaushik et al, 2020 that involves minimal media, 3 cultivation stages and media exchange via centrifugation. The producer screenings were performed in 96 deep-well plates with 500 μL volume.

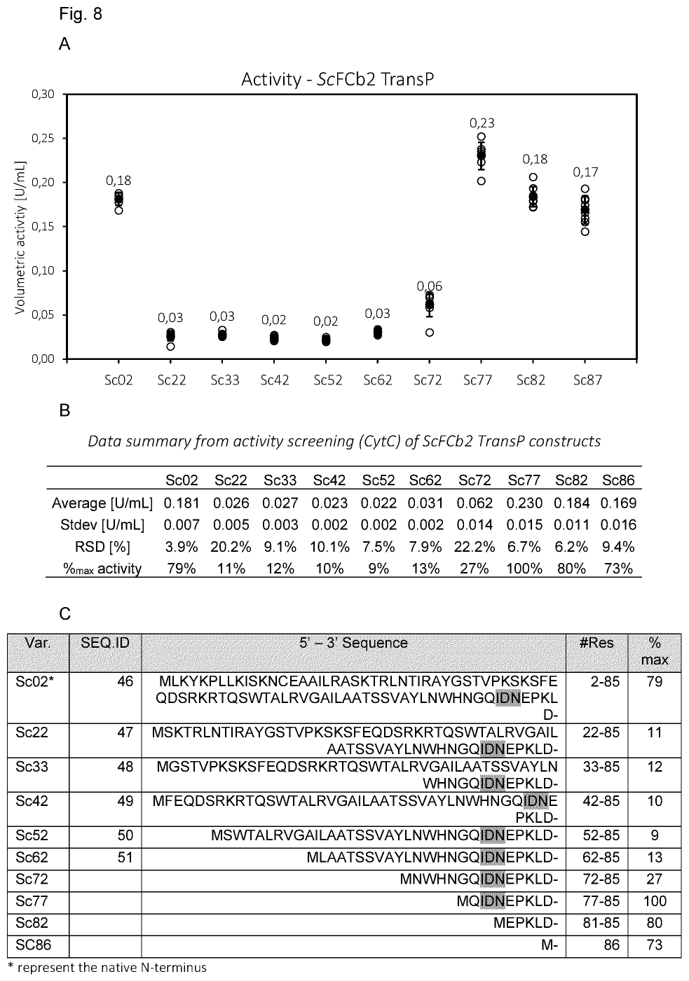

The following translocation peptide (TransP) variants, characterized by a different degree of truncation, were prepared by commercial DNA synthesis, and subsequently transformed into competent P. pastoris cells, relying on a methanol-inducible expression and an antibiotic resistance gene for selection (FIG. 8-10):
10×ScFCb2 TransP: Sc02, Sc22, Sc33, Sc42, Sc52, Sc62, Sc72, Sc77, Sc82, Sc86
10×WaFCb2 TransP: Wa02, Wa22, Wa28, Wa37, Wa47, Wa57, Wa62, Wa67, Wa72, Wa74
10×KmFCb2 TransP: Km02, Km22, Km34, Km42, Km52, Km62, Km72, Km77, Km82, Km87

### Cultivation and Expression

Was carried out following the detailed protocols provided in the publication by Kaushik et al., 2020 (Kaushik et al. 2020).

### Cell Disruption

Was carried out using the chemical protein extraction reagent “Y-Per”; the disruption protocol is based on the standard operating procedure and suggested adaptations by the supplier Fisher scientific (78991).
1. Centrifuge deep-well plates at 4000 rcf, 20° C. for 10 minutes.
2. Remove the supernatant carefully
3. Add 150 μL Y-per yeast solution per well and agitate at 340 rpm, 33° C. for 30 minutes. Close with a suitable lid to prevent evaporation.
4. Add 450 μL PBS buffer to help pelleting and to dilute for activity measurements
5. Pellet the debris at 3000 rcf, 20° C. for 10 minutes.
6. Transfer 20 μL of the supernatant to a fresh 96 well plate for activity analysis using the CytC assay.

### Enzyme Activity Measurement Using the CytC Assay

The determination of volumetric (per volume supernatant, mL) and specific (per protein mass, mg) activities of the FCb2 variants was carried out in a plate reader format using the established CytC assay with 5 mM L-lactate as has similarly been described for cellobiose dehydrogenase and glucose (Geiss A. F. et al., 2021).

In detail: enzyme activity was determined in a plate reader setup by following the reduction of 80 μM cytochrome c from equine heart at 550 nm (CytC, ε550 nm=19.6 mM−1 cm−1, enzyme factor=0.98) Enzyme factors (EF) were used to recalculate the raw reading in absorbance change per time (Abs min−1) in a 200 μL scale setup to volumetric activities expressed as U mL−1. Assay mixes were buffered with standard PBS (phosphate buffered saline) buffer and monitored for 300 s on an Infinite plate reader (Tecan) at the respective wavelength at 30° C. Where enzyme samples occurred in non-purified state, i.e. crude extracts or supernatants, activities were related to maximal activities in %. The protein concentrations of purified FCb2 variants were determined via the absorbance at 280 nm. The theoretical molar absorption coefficient ε280 nm was calculated with Expasy Prot-Param (Swiss Institute of Bioinformatics).

### Results

### Transp Scfcb2 (No His)

FIG. 8 shows that the ScFCb2 TransP constructs increase in volumetric activities (yields) until the TransP recognition motif, which is of the sequence “I-D-N” in ScFCb2, is reached. Measurements are of good quality and standard deviations reasonable.

Maximal activity (0.23 U/mL) can be found for Sc77, where only the “I/L/V-x-N/A/L” motif remains of the translocation peptide. This is in contrast with the crystal structure from the native ScFCb2 (1 FCB) where EPKLD forms the N-terminus.

### TransP KmFCb2 (No His)

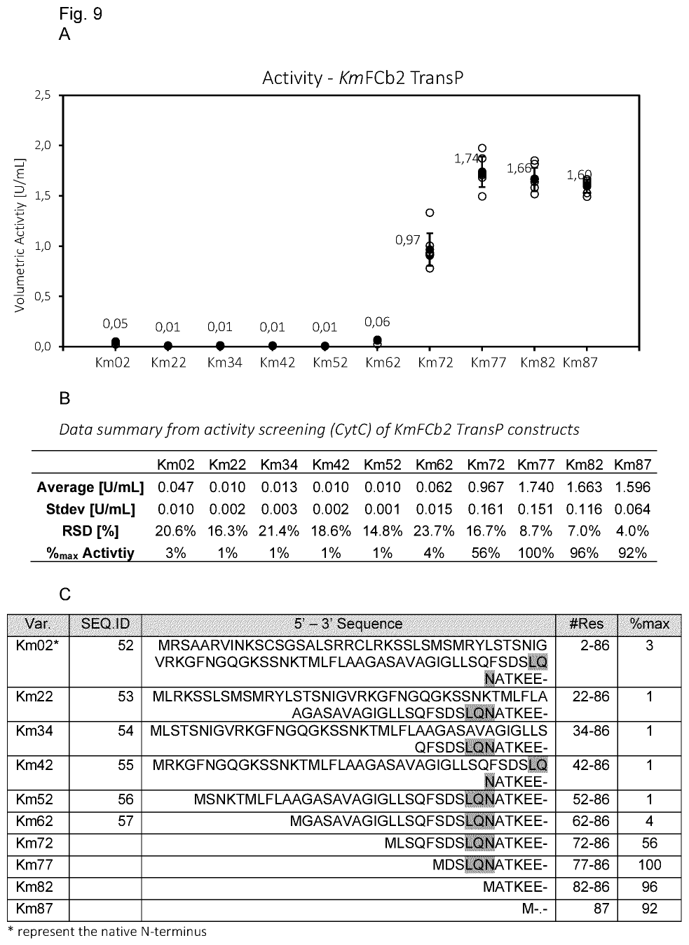

The KmFCb2 TransP constructs also increase in volumetric activities (yields) until the TransP recognition motif, which is of the sequence “I-Q-N” in KmFCb2, is reached (FIG. 9). Similar to ScFCb2, maximal activity (1.74 U/mL) can be found for Km77 where only the TransP recognition motif's “I-Q-N” forms the N-terminus.

Overall, activities are approx. 8-fold higher compared to ScFCb2, underlining the exceptional yields with KmFCb2. Contrasting to ScFCb2, no significant activity could be found for the native TransP construct Km02 (3% maximal).

### TransP WaFCb2 (No His)

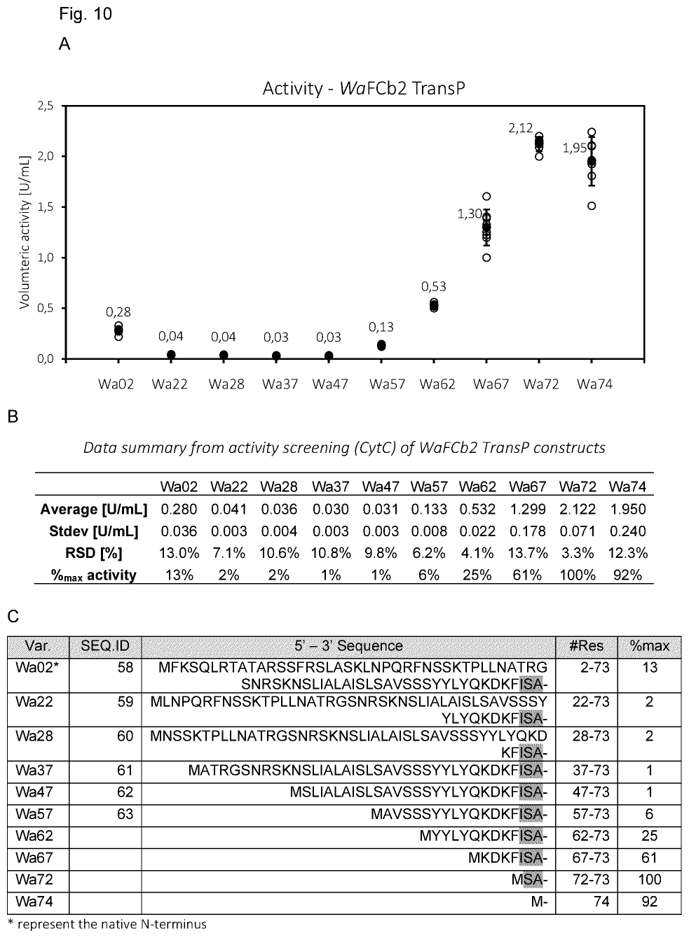

The WaFCb2 TransP constructs also increase in volumetric activities (yields) until the TransP recognition motif, which is of the sequence “I-S-A” in WaFCb2, is reached (FIG. 10). Maximal activity (2.12 U/mL) can be found for Wa72 where only parts of the “I-x-N” forms the N-terminus.

Overall activities are approx. 10-fold higher compared to the ScFCb2. Similar to KmFCb2 and contrasting to ScFCb2, only minor activities could be found with the native TransP (13% only).

### Conclusion

The previously tested and adapted screening protocol (Kaushik et al., 2020) utilizing 96 deep well plates and minimal media was employed to screen FCb2 TRansP variants. Generally, a mixed spectrum of results was obtained for the native TransP constructs:
For the native KmFCb2 comprising the TransP sequence, no activities could be yielded (3%)
For the native WaFCb2 comprising the TRansP sequence, minor activities were obtained (13%)
For the native ScFCb2 comprising the TransP sequence, significant activities are reported (79%)

Sequences with partial truncations did not yield good results and it seems likely that certain sequence features disturb proper processing or expression.

The prediction of the TransP motif “I/L/V-x-N/A/L” was accurate and its impact on activities and yields of FCb2 in the P. pastoris expression platform is immanent.

The data suggest that it is not necessary to remove the entire translocation peptide to facilitate high yields and successful expression, but sequences prior to that hinder good performance. Sequences where the translocation peptide was removed up to the “I/L/V-x-N/A/L” motif performed best for all three variants, KmFCb2, WaFCb2 and ScFCb2.

For ScFCb2, the highest volumetric activity in the given setup (FIG. 8) was shown for Sc77 carrying the “I/L/V-x-N/A/L”, one amino acid directly upstream of said motif, and a N-terminal Met. In total the signal peptide of Sc77 has 5 amino acids (the sequence “EPKLD” is the mature N-terminus as described elsewhere herein).

For KmFCb2, the highest activity in the given setup (FIG. 9) was shown for Km77 carrying the “I/L/V-x-N/A/L”, two amino acids directly upstream of said motif, and a N-terminal Met. In total the signal peptide of Km77 has 6 amino acids (the sequence “ATKEE” is the mature N-terminus as described elsewhere herein).

For WaFCb2, the highest activity in the given setup (FIG. 10) was shown for Wa72 carrying a truncated form of the “I/L/V-x-N/A/L” plus a N-terminal Met. In total the signal peptide of Wa72 has 3 amino acids (the sequence “DVPH” is the mature N-terminus as shown herein in FIG. 7).

For ScFCb2 and KmFCb2, the truncation of up to amino acid 77 of the native signal peptide resulted in the highest volumetric activity. In WaFCb2, the truncation of up to amino acid 72 of the native signal peptide resulted in the highest volumetric activity. Sequences where the entire translocation peptide was removed, including the “I/L/V-x-N/A/L” motif also worked well and displayed high yields and successful expression.

Volumetric activity drops considerably if the sequence of the native signal peptide upstream of the “I/L/V-x-N/A/L” motif comprises more than 9 amino acids of the native signal peptide. As shown in FIG. 8, the volumetric activity of Sc22, Sc33, Sc42, Sc52, and Sc62 is very low. Accordingly, as shown in FIG. 9, also the volumetric activity for Km02, Km22, Km34, Km42, Km52, and Km62 is also very low. And finally, also sequences Wa02, Wa22, Wa28, Wa37, Wa47, and Wa57 show very low volumetric activities (FIG. 10).

The results show that only a specific truncation pattern of the signal sequence leads to a functionally expressed FCb2 with a considerable yield/volumetric activity.

It was surprising that it is at all possible to express a functionally active FCb2 in the cytosol since it was specifically reported by Diekert K. et al. (2001) that the nascent Cytochrome C (CytC) of Saccharomyces cerevisiae is an apo-enzyme, lacking the heme cofactor and CytC is only activated after arriving in the mitochondrial intermembrane space (IMS) by interaction with a specialized lyase enzyme that attaches the heme. There are multiple parallels of CytC and FCb2 in yeast: they share a similar leader sequence architecture with a bipartite pre-sequence consisting of an N-terminal matrix targeting signal and C-terminal hydrophobic stretch, both have a non-covalent heme-cofactor, both occur in the mitochondrial intermembrane space, and both are part of the respiratory chain. Since both, CytC and FCb2, are dependent on a non-covalently bound heme cofactor, it would be assumed that FCb2 would also require the action of supporting enzymes located in the IMS to activate the catalytic activity. FCb2 activities outside the mitochondria have not been reported and the presence of FCb2 in the cytosol was also not reported so far. Therefore, the expression of a functionally active FCb2 which is not located into the mitochondrial intermembrane space was unpredictable and surprising. It was surprising that FCb2 does not need activation in the mitochondrial intermembrane space and that the FCb2 was active at all in the cytosol.

Due to the specific truncation of the signal peptide, the N-terminus of FCb2 is differently processed and the N-terminus of the herein expressed FCb2 is not the same as of the FCb2 naturally expressed in a yeast cell. As shown herein, the FCb2 has a Met at the N-terminus followed by the mature FCb2 sequence or alternatively has Met followed by a truncated native signal peptide. In general, the artificial modification of the N-terminus of proteins can have detrimental effects on the function of the proteins. For example, as reported by Petrović et al. (2018), the enzyme lytic polysaccharide oxygenase carries an N-terminal methylated methionine which is crucial for its activity. It was surprising that the FCb2 according to the invention is active despite modification of its mature N-terminus even only by addition of an artificial methionine. Also, in line with the prerequisite of a defined N-terminus, the effect of the herein provided truncation of the signal sequence of FCb2 is limited to the defined truncation of the invention—longer signal sequences lead to a rapid loss of function of the FCb2.

The artificial modification of a post-translational translocation process as in the case of FCb2 shown herein by altering the signal peptide of the protein could result in loss of activity since denaturation, degradation, or controlled disintegration could occur at many stages of the process. In general, the expression of a mature mitochondrial intermembrane space protein always relies on the presence of an intact and stable pre-version that passages the route of endoplasmic reticulum (ER), cytosol, mitochondrial matrix, mitochondrial intermembrane space and relies also on interactions of the later cleaved signal sequence with the rest of the later “mature” protein.

As described in Beasley et al. (1993) the signal sequence of FCb2 is bipartite, wherein the N-terminal region of the signal peptide is responsible for translocation into the mitochondrial matrix as a matrix-targeting signal. The C-terminal region of the signal peptide is necessary for sorting into the intermembrane space. In the specific example of S. cerevisiae FCb2, the N-terminal matrix-targeting signal comprises amino acids 1 to 32 of the native, full length signal sequence given in SEQ ID NO: 3, the signal peptide for sorting into the intermembrane space comprises amino acids 33 to 80 of the native signal sequence given in SEQ ID NO: 3. Although it was already surprising that the expression of functional FCb2 in the cytosol was generally possible at all, in light of the bipartite nature of the signal peptide, it was even more surprising that this functional expression was not possible by simply deleting the matrix-targeting signal. Instead, the data clearly show that a further truncation of the signal peptide is needed. Although due to deletion of the matrix-targeting signal of FCb2, these FCb2 must stay in the cytosol since the signal for translocation into the mitochondrial matrix is not present anymore, there was no or only marginal activity found for FCb2s comprising a truncation of this matrix-targeting signal only. Using the herein shown specific truncated signal peptide of FCb2, a functional FCb2 could be expressed in the cytosol.

The findings show that FCb2 sequences with longer signal peptide sequences as the truncated signal peptides as shown herein are not functionally active in the cytosol. The specific truncation pattern of the invention gives guidance on which sequence portion of the native signal sequence allows to discriminate future active from inactive FCb2.

In summary, N-terminal truncation of FCb2 sequences, up to or including the TransP motif “I/L/V-x-N/A/L”, significantly increases expression rate, activities, and yields.

This effect was shown for three different FCb2 sequences from different organisms: S. cerevisiae, K. marxianus, and W. anomalus. In the following Examples 3 and 4 it is shown that these findings from Example 2 and the herein provided truncation of the signal peptide can be used to predict functional expression in the cytosol of a FCb2 from other organisms.

### Example 3: Expression of His-Tagged FCb2 Variants

This example summarizes and evaluates the results of the production process (expression and purification) and characterization of the FCb2 TransP-His constructs from S. cerevisiae (ScFCb2), K. marxianus (KmFCb2), W. anomalus (WaFCb2) and C. glabrata (CangFCb2). All containing an N-terminal His₈-tag and transP sequences of three different lengths. The variants were prepared by commercial DNA synthesis, subsequently transformed into competent P. pastoris cells relying on a methanol-inducible expression and an antibiotic resistance gene for selection:

| | |
|---|---|
| TransP, full + His: | Sc02-His |
| | (SEQ ID NO: 17) |
| | Km02-His |
| | (SEQ ID NO: 18) |
| | Wa02-His |
| | (SEQ ID NO: 19) |
| | Cang02-His |
| | (SEQ ID NO: 20) |
| TransP. - MPP + His: | Sc33-His |
| | (SEQ ID NO: 21) |
| | Km34/His |
| | (SEQ ID NO: 22) |
| | Wa28/His |
| | (SEQ ID NO: 23) |
| TransP, negative + His: | Sc82/His |
| | (SEQ ID NO: 24) |
| | Km82/His |
| | (SEQ ID NO: 25) |
| | Wa74/His |
| | (SEQ ID NO: 26) |
| | Cang80/His |
| | (SEQ ID NO: 27) |

### Cultivation, Cell Disruption and Purification

Growth of cells and induction of enzyme expression was carried out as is described in the publication by Geiss et al. 2021 with minor deviations. After the cultivation stage, roughly 13 g cell pellet were obtained from 600 mL of culture and subjected to cell disruption in 4 cycles using a 30 mL french cell press homogenizer operating at 1500 bar external pressure. The so yielded cell debris was centrifuged at 10 000 rcf for 30 minutes and the obtained supernatant, the crude extract, was purified as is described in the publication, essentially using an immobilized metal affinity chromatography (IMAC) strategy.

### Enzyme Activity Measurement Using the CytC Assay

The determination of volumetric (per volume supernatant, mL) and specific (per protein mass, mg) activities was carried out in a plate reader format using the established CytC assay with 5 mM L-lactate as has similarly been described for cellobiose dehydrogenase and glucose (Geiss A. F. et al., 2021). In detail: enzyme activity was determined in a plate reader setup by following the reduction of 80 μM cytochrome c from equine heart at 550 nm (CytC, ε550 nm=19.6 mM−1 cm−1, enzyme factor=0.98) Enzyme factors (EF) were used to recalculate the raw reading in absorbance change per time (Abs min−1) in a 200 μL scale setup to volumetric activities expressed as U mL−1. Assay mixes were buffered with standard PBS (phosphate buffered saline) buffer and monitored for 300 s on an Infinite plate reader (Tecan) at the respective wavelength at 30° C. The protein concentrations of purified FCb2 variants were determined via the absorbance at 280 nm. The theoretical molar absorption coefficient ε280 nm was calculated with Expasy Prot-Param (Swiss Institute of Bioinformatics).

### Purity

SDS PAGE of the purified TransP-His constructs was carried out:
All samples were diluted to ˜0.4 mg/mL in sterile hq-H2O
10 μL diluted enzyme sample were mixed with 10 μL 2× Laemmli buffer heated at 99° C. for 5 min and then put on ice for 5 min
14 μL sample were loaded into the Mini-PROETAN TGX gel pockets
5 μL precision plus unstained protein marker (Bio-Rad) as reference
The gel is run at 90 V for 5 min, then at 150 V for ˜50 min
Visualization of protein band was realized with UV illumination

### Results

Results of the expression and purification process of all TransP-His constructs are summarized in Table 1.

TABLE 1
Summary table of TransP-His FCb2 constructs

| | Crude | activity | | total | Total | specific | mg | |
|---|---|---|---|---|---|---|---|---|
| | extract | lost | Yield | enzyme | Units | activity | enzyme/g | RZ- |
| Enzyme | [total U] | [%] | [%] | [mg] | [U] | [U/mg] | cell pellet | value |
| Sc02-His | 37 | 100 | — | — | — | — | — | — |
| Sc33-His | 74 | 80 | 0.34 | 0.78 | 0.25 | 0.32 ± 0.01 | 0.06 | 0.17 |
| Sc82-His | 107 | 56 | 108 | 4.53 | 116 | 26 ± 3.3 | 0.34 | 1.34 |
| Km02-His | 150 | 100 | 3.2 | 1.91 | 4.8 | 2.5 ± 0.33 | 0.15 | 0.73 |
| Km34-His | 675 | 86 | 15.2 | 11.9 | 102 | 8.6 ± 1.3 | 0.86 | 1.13 |
| Km82-His | 2052 | 6 | 153 | 66.3 | 3145 | 47 ± 11 | 5.06 | 1.56 |
| Wa02-His | 324 | 94 | — | — | — | — | — | — |
| Wa28-His | 100 | 97 | 0.4 | 0.32 | 0.40 | 1.2 ± 0.1 | 0.031 | 0.04 |
| Wa74-His | 121 | 62 | 37 | 2.55 | 45 | 18 ± 5.0 | 0.21 | 1.52 |
| Cang02-His | 55 | 91 | — | — | — | — | — | — |
| Cang80-His | 1236 | 20 | 67 | 15.7 | 827 | 53 ± 1.1 | 1.28 | 1.45 |

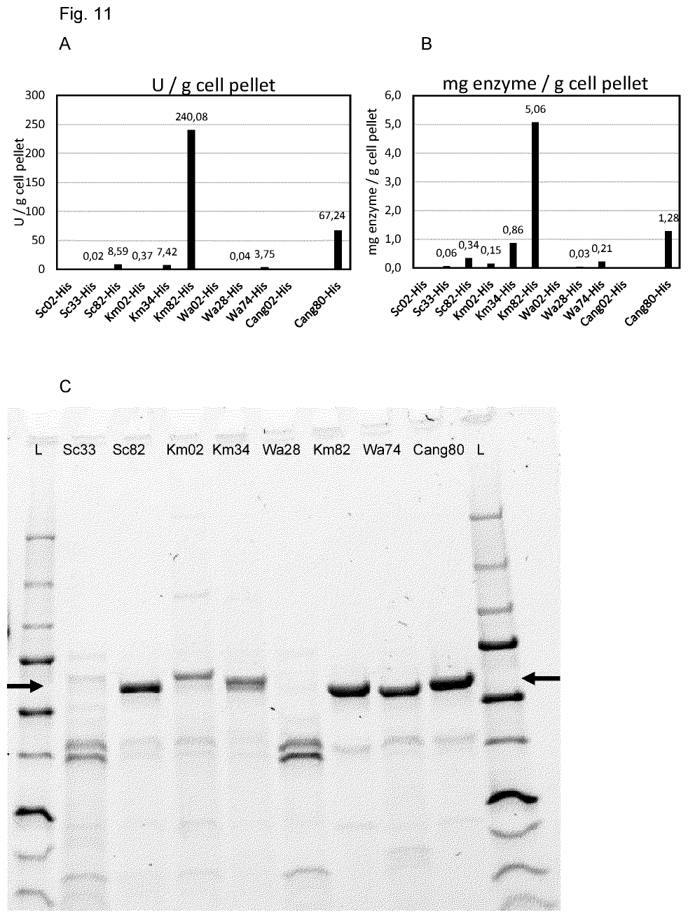

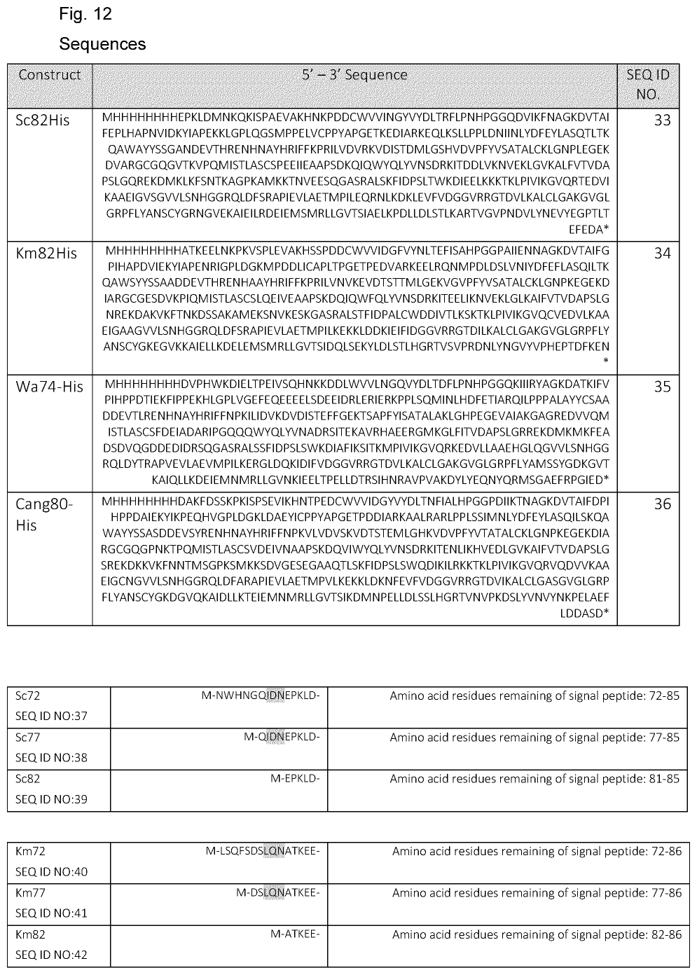

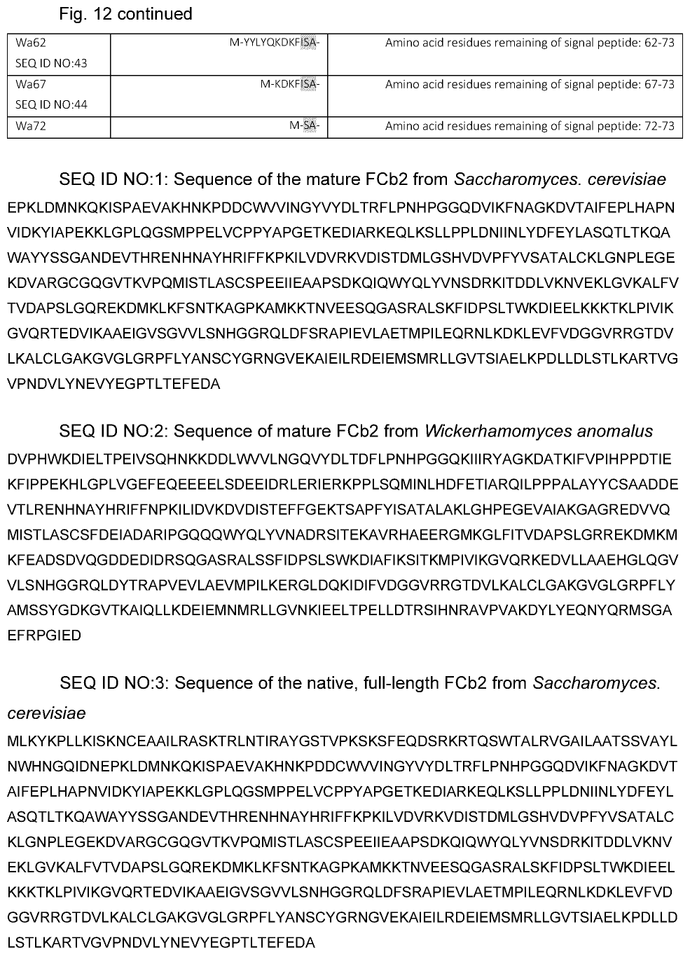

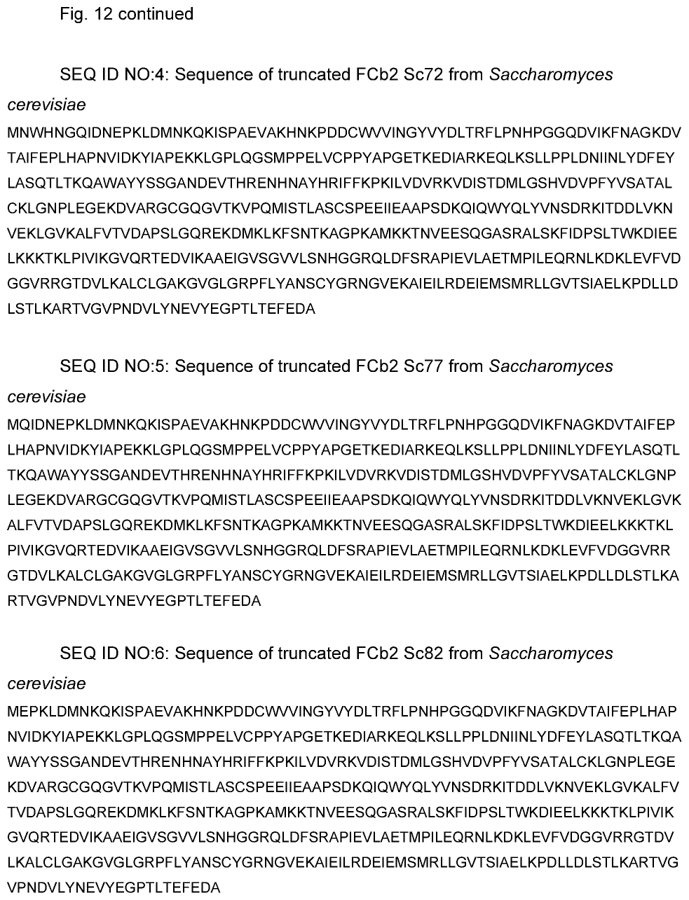

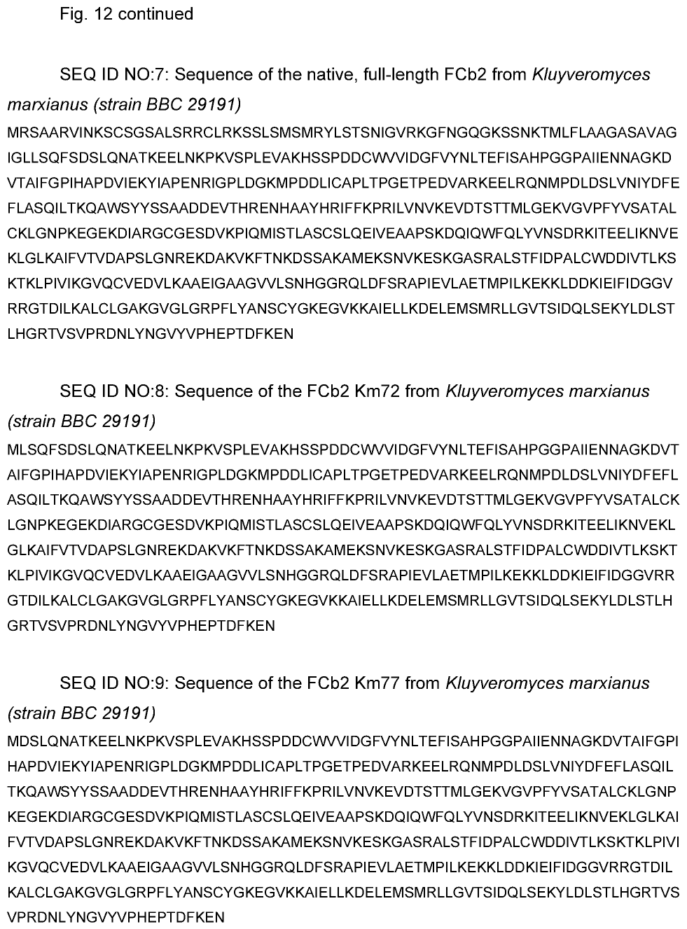

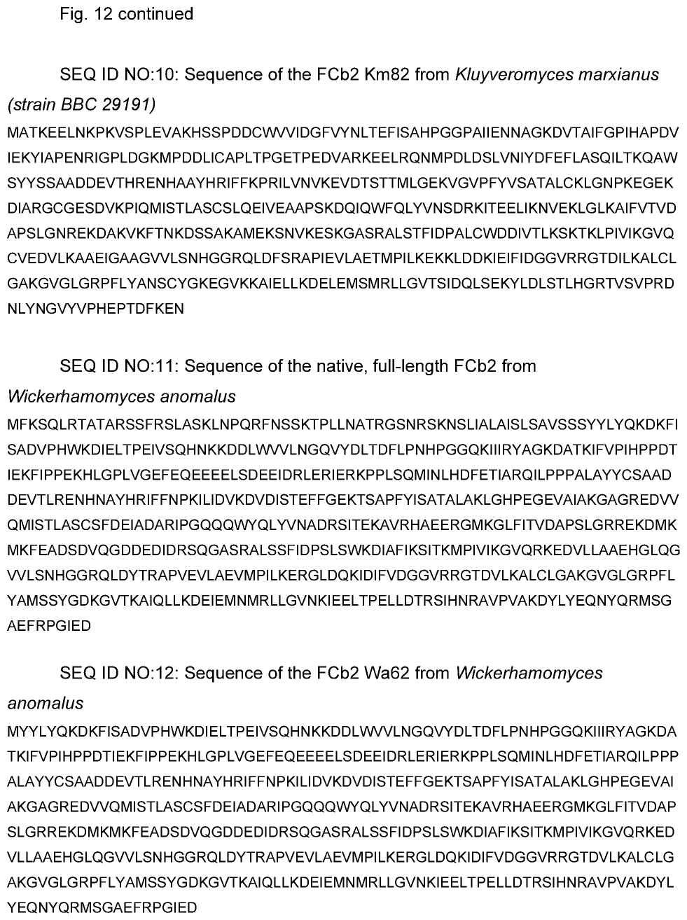

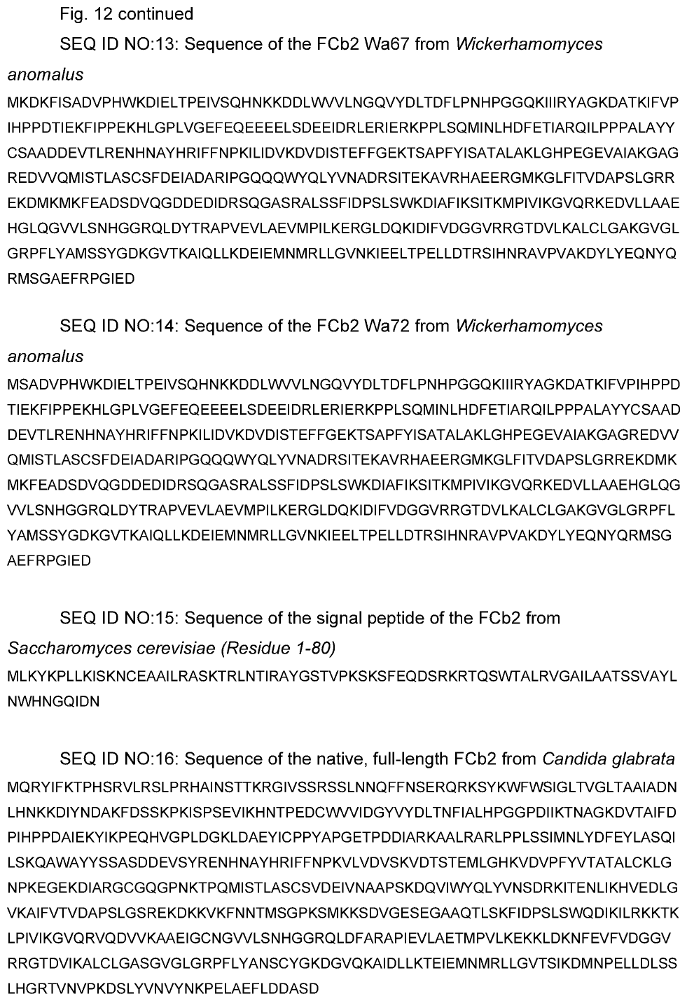

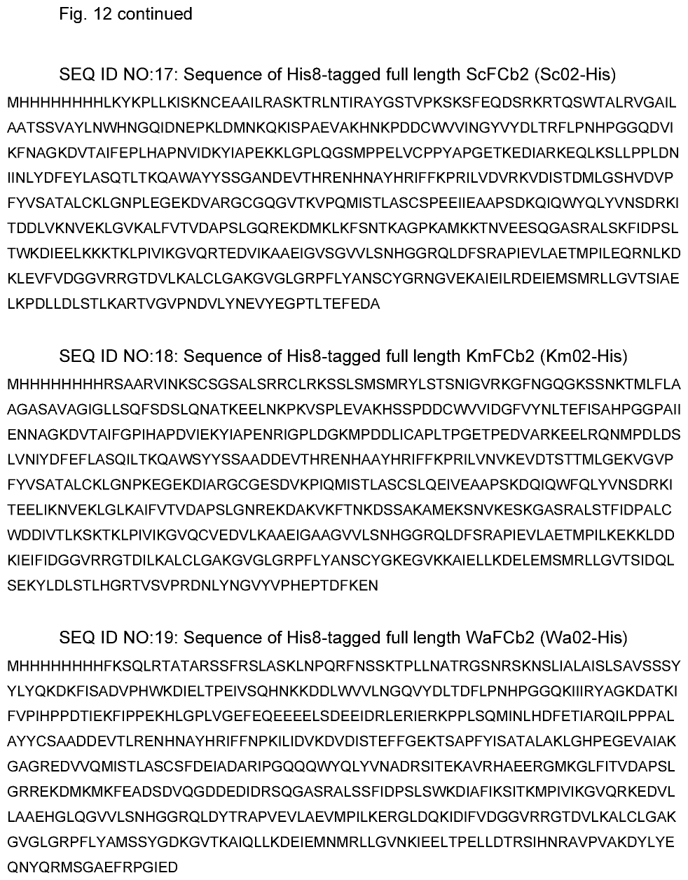

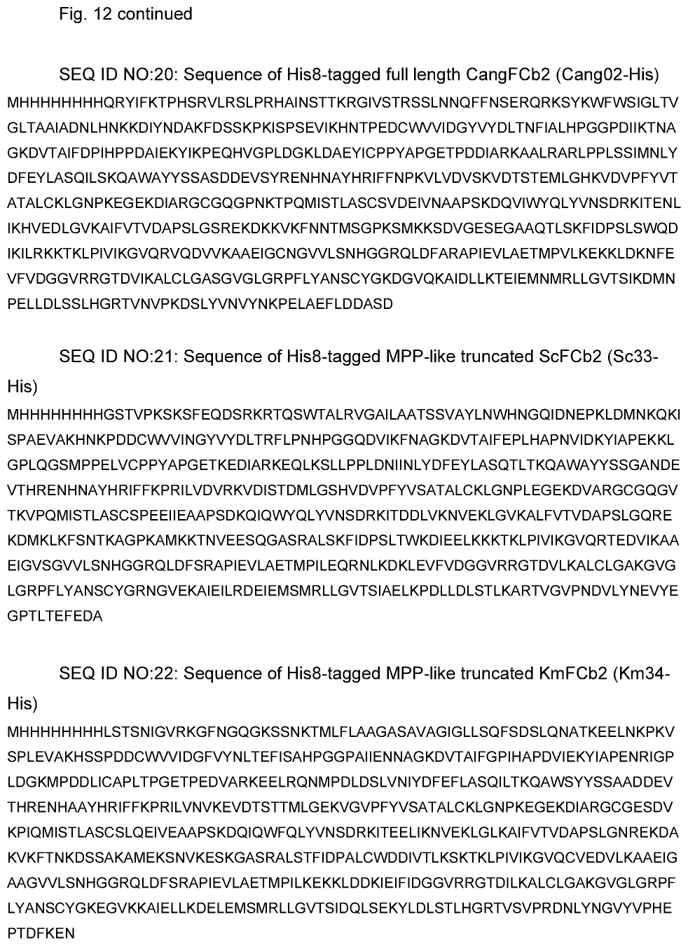

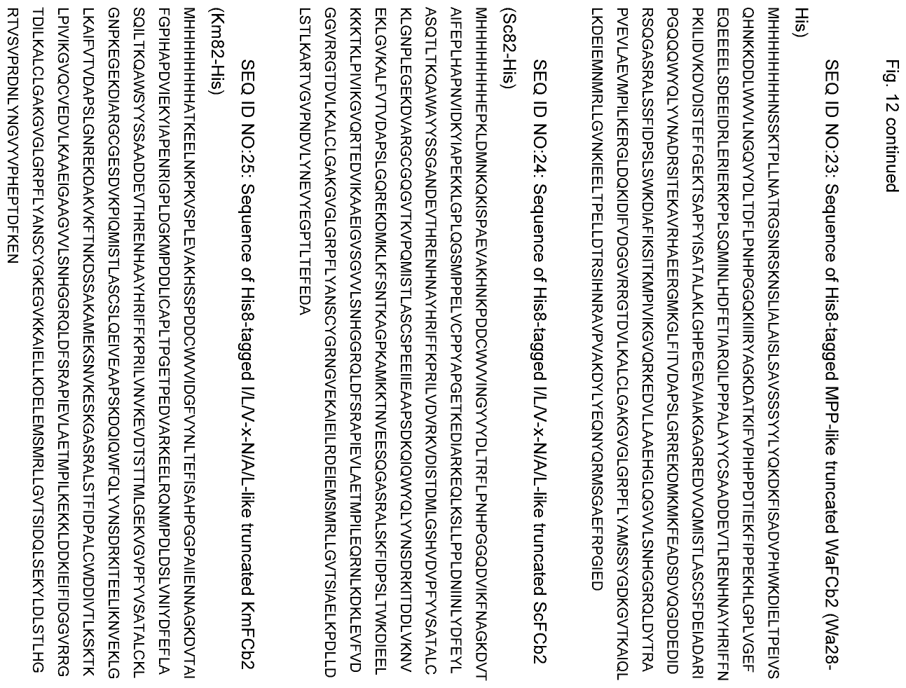

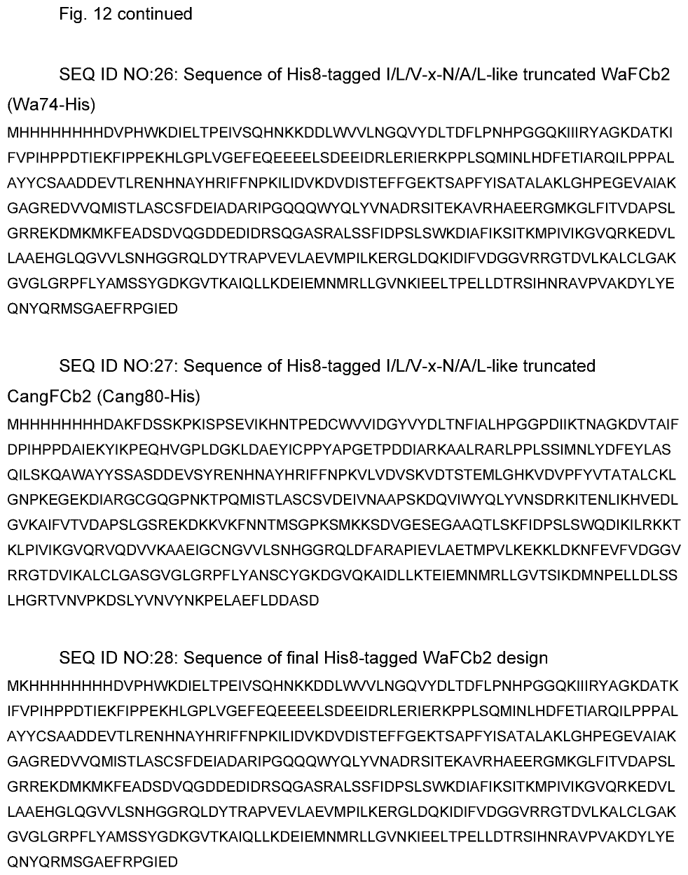

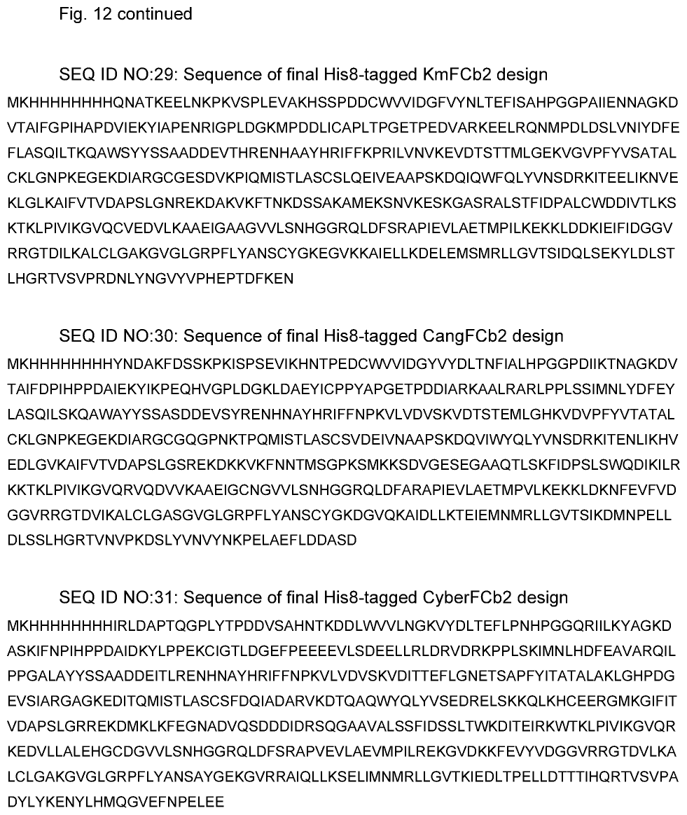

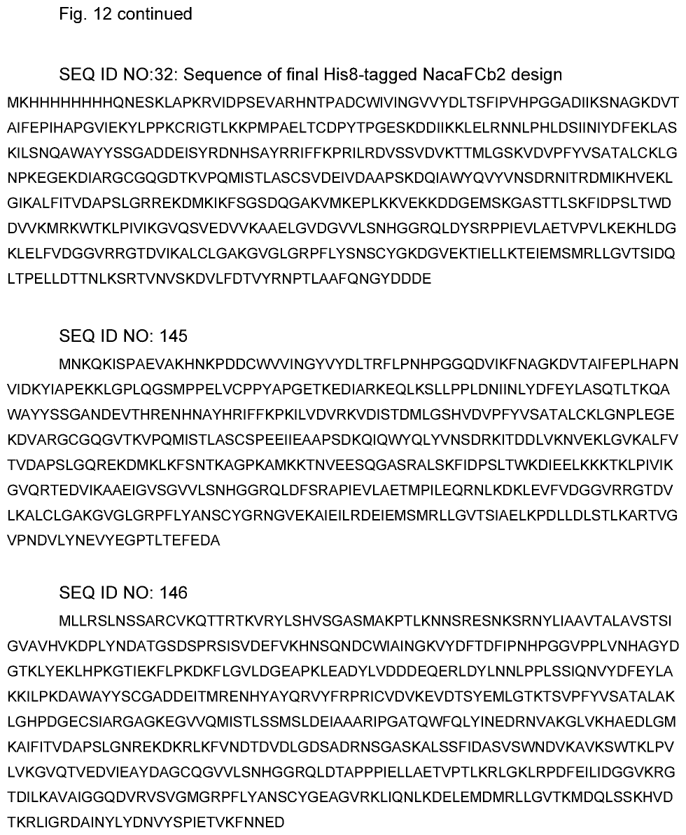

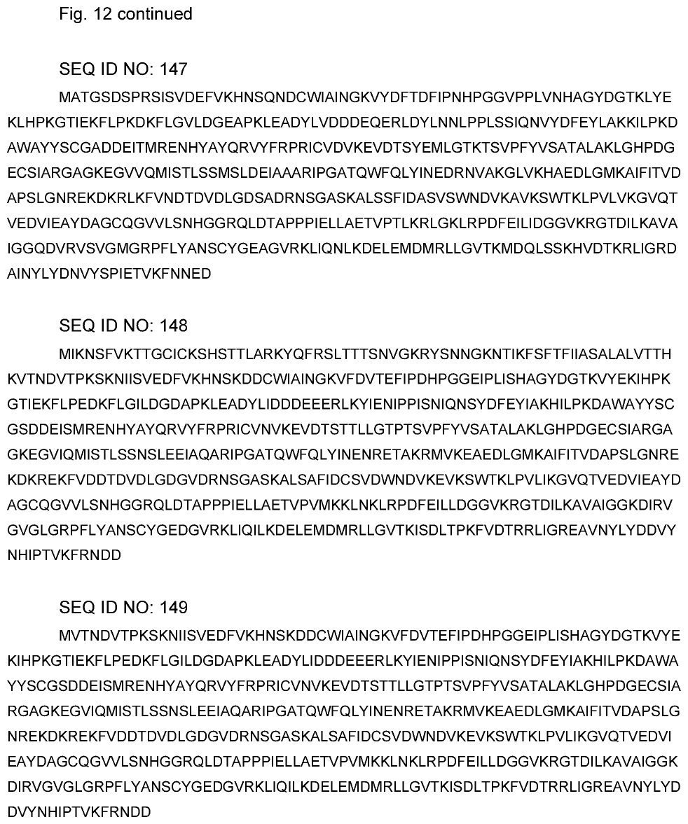

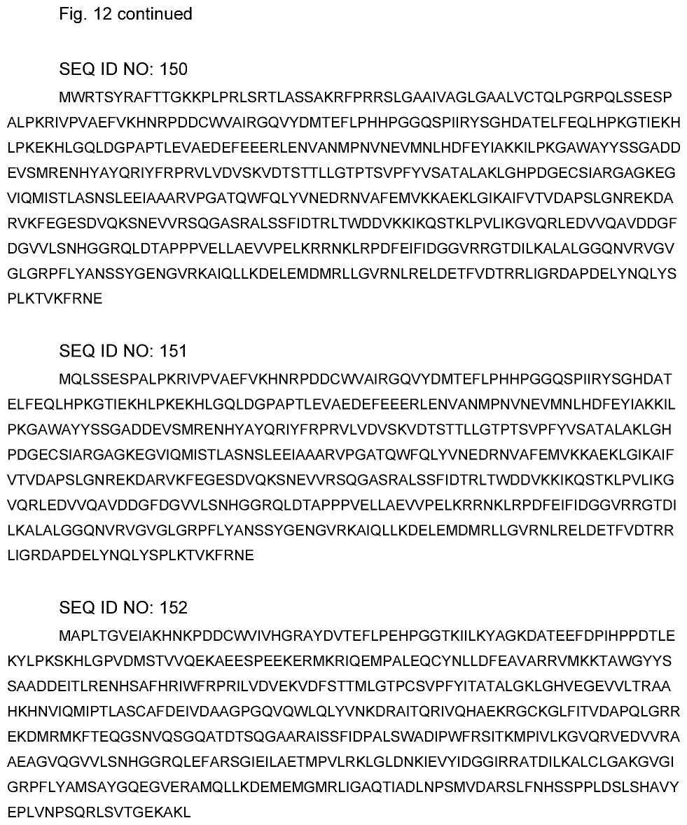

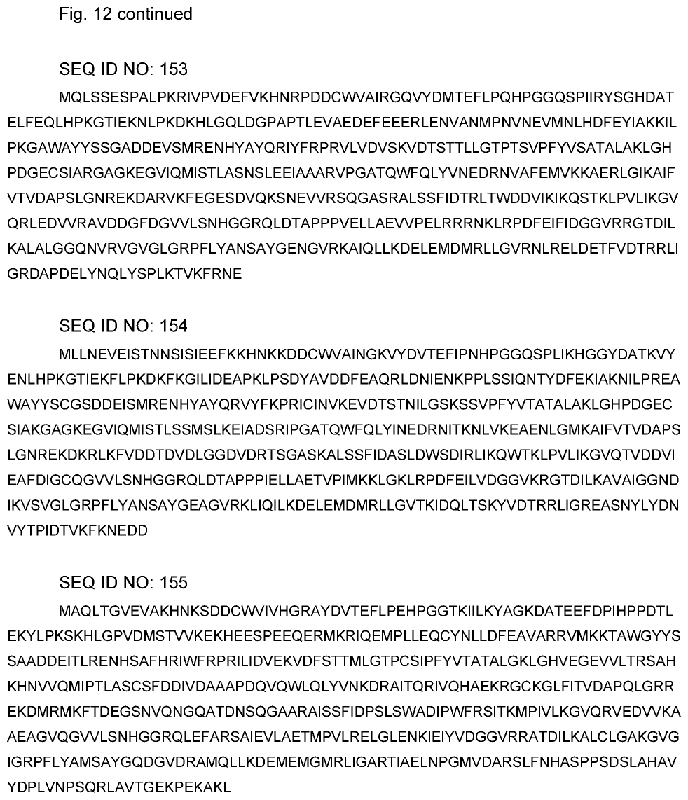

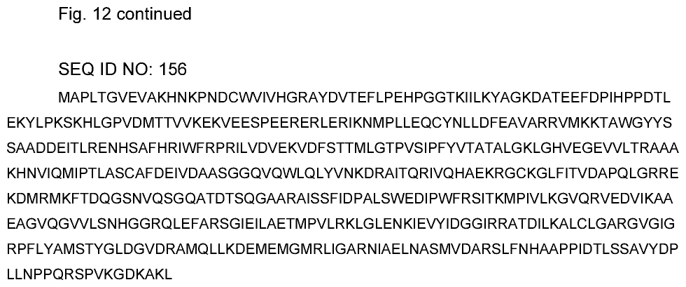

In summary, Table 1 and FIG. 11-A/B/C show for all four His-tagged variants (ScFCb2, KmFCb2, WaFCb2 and CangFCb2): The shorter the translocation peptide sequence, the higher the expression and purification yield and the higher the specific activity and RZ-value (A_{412 nm}/A_{276 nm}) an indicator for the spectral purity of the FCb2 enzyme by means of the absorbance of the heme-cofactor.

S. cerevisiae (ScFCb2)

TransP Sc02-His FCb2: His-tagged ScFCb2 could not be expressed when comprising the full-length translocation peptide. No enzyme could be obtained

TransP Sc33-His FCb2: containing the trans P-MPP and an additional N-terminal His8-tag, ScFCb2 could not be expressed and purified sufficiently via IMAC.
0.019 U and 0.06 mg enzyme/g cell pellet (Purification yield: 0.34%)
decreased specific activity with Cyt c; decreased RZ-value
SDS PAGE: shows impurities or degradation products of the enzyme
   no pure or fully expressed enzyme obtained

TransP Sc82-His FCb2: containing no translocation peptide but an additional N-terminal His8-tag, ScFCb2 was expressed and could be successfully purified via IMAC
8.6 U and 0.34 mg enzyme/g cell pellet (Purification yield: 109%)
Specific activity: 25.7 U/mg; RZ-value: 1.34
SDS PAGE: intense band at ˜58 kDa shown (FIG. 11-C)
   Fully expressed and active enzyme obtained

As shown in Example 2, full-length ScFCb2 (comprising the entire translocation peptide) could be expressed. However, when an N-terminal His-tag is added, full-length ScFCb2 is no longer expressed. As shown in this example, when the translocation peptide is deleted, fully active His-tagged ScFCb2 can successfully be expressed and purified.

K. marxianus (KmFCb2)

TransP Km02-His FCb2: with the complete translocation peptide and an additional N-terminal His8-tag, KmFCb2 could not be expressed and purified via IMAC sufficiently according to the used protocols
0.37 U and 0.15 mg enzyme/g cell pellet (Purification yield: 3%)
Decreased specific activity with Cyt c; decreased RZ-value
SDS PAGE: band at ˜60 kDa, but also some impurities (FIG. 11-C)
   No pure and not very active enzyme obtained

TransP Km34-His FCb2: containing the trans P-MPP and an additional N-terminal His8-tag, KmFCb2 could be expressed but not purified via IMAC sufficiently according to the used protocols
7.4 U and 0.86 mg enzyme/g cell pellet (Purification yield: 15%)
Decreased specific activity with Cyt c; decreased RZ-value
SDS PAGE: shows intense band at ˜60 kDa (FIG. 11-C)
   Pure enzyme obtained but shows decreased activity

TransP Km82-His FCb2: containing no translocation peptide but an additional N-terminal His8-tag, KmFCb2 was successfully expressed and purified via IMAC according to the used protocols
240 U and 5 mg enzyme/g cell pellet (Purification yield: 153%)
Specific activity: 47.4 U/mg; RZ: 1.56
SDS PAGE: intense band at ˜58 kDa shown (FIG. 11-C)
   Fully expressed and active enzyme obtained
      W. anomalus (WaFCb2)

TransP Wa02-His FCb2: with the complete translocation peptide and an additional N-terminal His8-tag, WaFCb2 could not be expressed and further not purified according to the used protocols
No enzyme obtained

TransP Wa28-His FCb2: containing the trans P-MPP and an additional N-terminal His8-tag, WaFCb2 could not be expressed and purified via IMAC sufficiently according to the used protocols
0.04 U and 0.03 mg enzyme/g cell pellet (Purification yield: 0.4%)
Decreased specific activity with Cyt C; decreased RZ-value
SDS PAGE: shows impurities or degradation products (FIG. 11-C)
   No pure or fully expressed enzyme obtained

TransP Wa74-His FCb2: containing no translocation peptide but an additional N-terminal His8-tag, WaFCb2 could be expressed and purified via IMAC according to the used protocols
8.3 U and 0.21 mg enzyme/g cell pellet (Purification yield: 37%)
Specific activity: 17.5 U/mg; RZ-value: 1.52
SDS PAGE: intense band at ˜58 kDa shown (FIG. 11-C)
   Fully expressed and active enzyme obtained
      C. glabrata (Can FCb2)

TransP Cang02-His FCb2: with the complete translocation peptide and an additional N-terminal His8-tag, CangFCb2 could not be expressed and further not purified according to the used protocols
no enzyme obtained

TransP Cang80-His FCb2: containing no translocation peptide but an additional N-terminal His8-tag, CangFCb2 could be expressed and purified via IMAC successfully according to the used protocols
67 U and 1.3 mg enzyme/g cell pellet (Purification yield: 67%)
Specific activity: 53 U/mg; RZ-value: 1.45
SDS PAGE: intense band at ˜58 kDa shown (FIG. 11-C)
   Fully expressed and active enzyme obtained

### Conclusion

Definite effect of the N-terminal sequence on FCb2 expression and purification yield (and specific activity) shown:

TransP, full+His (Sc02, Km02, Wa02, Cang02): FCb2s could not be fully expressed and further not purified via IMAC accordingly.

TransP, −MPP+His (Sc33, Km34, Wa28): FCb2s are only poorly expressed and purified via IMAC with decreased specific activity and different RZ-value.

TransP, negative+His (Sc82, Km82, Wa74, Cang80): FCb2s could be successfully expressed and purified via IMAC.

Furthermore, it is shown that the findings relating to the truncated signal peptide of Example 2 can be used to predict a functional expression of FCb2 in the cytosol since Cang80 with a truncation of the signal peptide according to the invention could be functionally expressed with high activity and yield in the cell pellet (FIG. 11).

### Example 4: Yields and Biochemical Properties of New FCb2 Constructs Comprising Truncated Translocation Peptide Sequences

The selection of new FCb2 sequences was based on the bioinformatic analysis of phylogenetic relations and predictions of thermal stability using available online tools: BlastP (sequence search, https://blast.ncbi.nlm.nih.gov/), NGPhylogeny (phylogeny, https://ngphylogeny.fr/), SWISS-MODEL (structure prediction, https://swissmodel.expasy.org/) and SCooP (stability prediction, http://babylone.ulb.ac.be/SCooP). Expression constructs were designed according to the previous findings in Example 3, where the native N-terminus was truncated till the conserved I/L/V-x-N/A/L motif and a His8-tag was added after the initial Methionine.

Intracellular expression of the variants was again carried out in the shaking flask format and the obtained cell pellets were stored frozen before cell disruption and purification via IMAC as is described in Example 3. Variants were analysed similarly to the processes described in Example 3.

This example summarizes the results (expression, purification and biochemical characterization) of new FCb2 variants comprising truncated signal sequences up to the I/L/V-x-N/A/L motif.
Wickerhamomyces anomalus FCb2 (WaFCb2, SEQ ID NO: 28)
Kluyveromyces marxianus FCb2 (KmFCb2, SEQ ID NO: 29)
Candia glabrata FCb2 (CangFCb2, SEQ ID NO: 30)
Cyberlindnera fabianii FCb2 (CyberFCb2, SEQ ID NO: 31)
Naumovozyma castelli FCb2 (NacaFCb2, SEQ ID NO: 32)

### Results

Results of yields and biochemical characterization of all new FCb2 variants are summarized in Table 2: (WaFCb2 (His): SEQ ID NO 28; KmFCb2 (His): SEQ ID NO 29; CangFCb2 (His): SEQ ID NO 30; CyberFCb2 (His): SEQ ID NO 31; NacaFCb2 (His): SEQ ID NO 32)

TABLE 2
Result table: Expression and purification

| | cell | Crude | activity | totol | Total | specific | mg | |
|---|---|---|---|---|---|---|---|---|
| | pellet | extract | lost | enzyme | Units | activity | enzyme/g | RZ- |
| Enzyme | [g] | [total U] | [%] | [mg] | [U] | [U/mg] | cell pellet | value |
| WaFCb2 | 12.0 | 312 | 43 | 20.9 | 252 | 12.1 | 1.74 | 1.64 |
| KmFCb2 | 11.4 | 2256 | 3 | 56.4 | 4982 | 88 | 4.95 | 1.57 |
| CangFCb2 | 11 | 1885 | 4 | 30.5 | 1325 | 44 | 2.77 | 1.44 |
| CyberFCb2 | 11.4 | 48.3 | 91 | 1.67 | 72 | 43 | 0.146 | 1.3 |
| NacaFCb2 | 10.5 | 44.0 | 25 | 2.3 | 20.5 | 8.8 | 0.219 | 1.2 |

In summary, all variants show excellent yields and specific activities that make them fit for production. It is shown in Example 4 that the truncation of the signal peptide according to the invention can be used for predicting the expression of FCb2 sequences as functional enzymes with high yield and specific activity in the cytosol of the cell. The data show that the functional expression in the cytosol of all shown FCb2 is enabled due to the technical feature of the specific truncation of the signal peptide according to the invention.

### Example 5: Direct Electron Transfer of FCb2 Variants Modified Carbon Paste Electrodes

WaFCb2, KmFCb2, CangFCb2 were immobilized on commercial carbon paste electrodes (type DRP-C110; Metrohm/DropSens) following a twostep protocol adapted from Geiss et al. (2021). First, the electrodes were submersed in a 1% (v/v) solution of ethylene glycol diglycidyl ether (EGDGE; Polysciences) dissolved in 0.1 M NaOH for 1 h at 60° C. After a washing step with water and drying with nitrogen, 1 μL of enzyme solution (10 mg mL⁻¹) was applied to the working electrode and dried for one hour at room temperature in presence of silica gel. Electrochemical readouts of the modified working electrodes were done using a potentiostat (EmStat3, PalmSens) and a screen-printed carbon counter and an Ag|AgCl (0.14 M NaCl) pseudo reference electrode. Current responses to increasing lactate concentrations were measured for sensors vertically immersed in 50 mM potassium phosphate buffer containing 8 g L⁻¹ NaCl and 0.2 g L⁻¹ KCl, pH 7.4, at 37° C. and at an applied potential of +0.2 V versus the pseudo reference electrode. Current densities were calculated by relating the currents to the enzyme coated working electrode area.

### Results

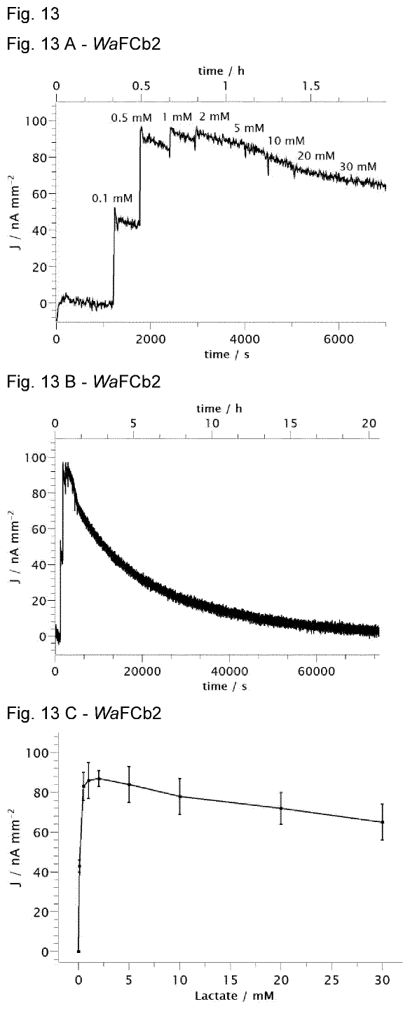

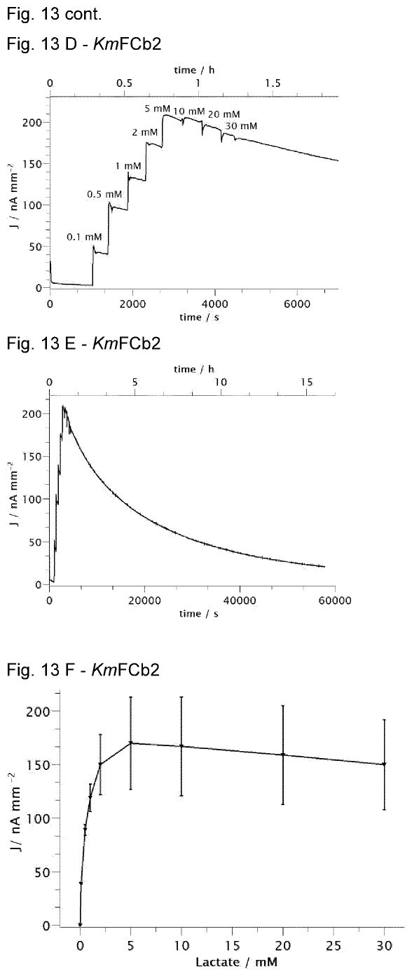

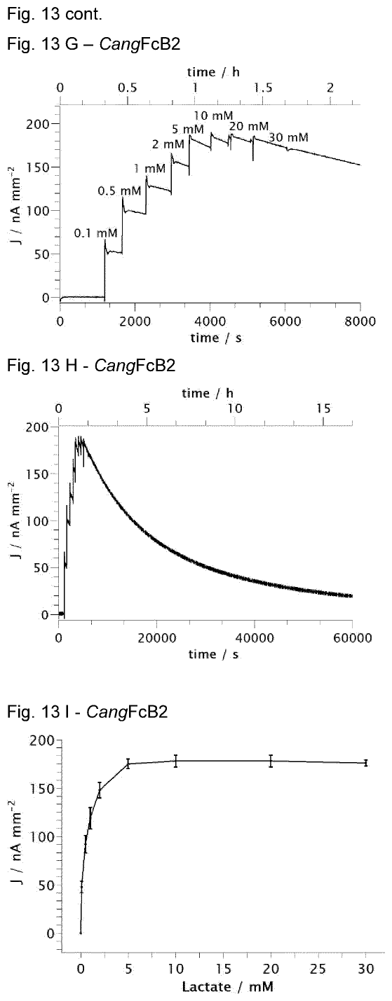

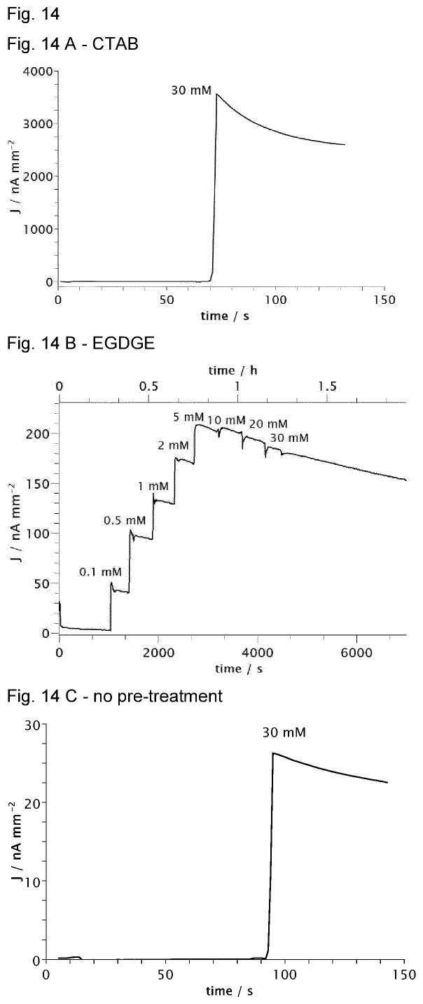

All tested FCb2 variants show increasing sensor signals with increasing lactate concentration, saturate in the low mM range and decrease over time (see FIG. 13). This can be expected since likely inactivation or desorption of the enzyme layer from the electrode surface progresses with time and no counteracting stabilizing measures like crosslinking or membranes were applied. Sensor signals originate from a direct electron transfer between enzyme and electrode as no mediator is present. Differences in sensor currents among the variants might originate from different enzyme activities and varying capabilities of direct electron transfer which is governed by (different) enzyme structures and orientations on the electrode surface.

### Example 6: Direct Electron Transfer to Pre-Modified Carbon Paste Electrodes

To improve the DET the surface of commercial carbon paste electrodes (type DRP-C110; Metrohm/DropSens) were pre-modified in two ways:

1: A cationic detergent cetyltrimethylammonium bromide (CTAB) was applied by dipping the electrode into a 0.2% (w/v) solution for 30 min, then rinsed with water, and dried in nitrogen.

2: Ethylene glycol diglycidyl ether (EGDGE) was applied following a twostep protocol adapted from Geiss et al. (2021). First, the electrodes were submersed in a 1% (v/v) solution of (EGDGE; Polysciences) in 0.1 M NaOH for 1 h at 60° C., then rinsed with water, and dried in nitrogen.

Control: No pre-treatment. Electrodes were used as delivered.

KmFCb2 was immobilized on the pre-treated electrodes by adding 1 μl of a 10 mg mL⁻¹ solution and dried for 1 h at room temperature in presence of silica gel. Electrochemical readouts of the modified working electrodes were done using a potentiostat (EmStat3, PalmSens), a carbon paste counter electrodes and a Ag|AgCl (0.14 M NaCl) pseudo reference electrode. Current responses to increasing lactate concentrations were measured for sensors vertically immersed in 50 mM potassium phosphate buffer containing 8 g L⁻¹ NaCl and 0.2 g L⁻¹ KCl, pH 7.4, at 37° C. and at an applied potential of +0.2 V versus the pseudo reference electrode. Current densities were calculated by relating the currents to the amount of enzyme per working electrode area.

### Results

KmFCb2 immobilized on pre-modified carbon paste electrodes can yield high current densities of over 3000 nA mm⁻² for 30 mM lactate. Currents originate from unmediated electron transfer, as no mediator is present, while the current density is modulated by the type of pre-modification; see FIG. 14.

### Example 7: Direct Electron Transfer on Gold and Carbon Paste Electrodes

The example demonstrates the capability of the produced FCb2 variants to perform direct electron transfer (DET) to conductive materials relevant for biosensors such as gold or carbon paste.

KmFCb2 was immobilized on gold (Zensor R&D co., Ltd) or carbon paste working electrodes (type DRP-C110; Metrohm/DropSens) by adding 1 μl of a 10 mg mL⁻¹ enzyme solution and drying it for 1 h at room temperature in presence of silica gel. Electrochemical readouts of the modified working electrodes were done using a potentiostat (EmStat3, PalmSens), a gold or carbon paste counter electrode and an Ag|AgCl (0.14 M NaCl) pseudo reference electrode. Current responses after adding 30 mM lactate were measured for sensors vertically immersed in 50 mM potassium phosphate buffer containing 8 g L⁻¹ NaCl and 0.2 g L⁻¹ KCl, pH 7.4, at 37° C. and at an applied potential of +0.2 V versus the pseudo reference electrode. Current densities were calculated by relating the currents to the enzyme coated working electrode area.

### Results

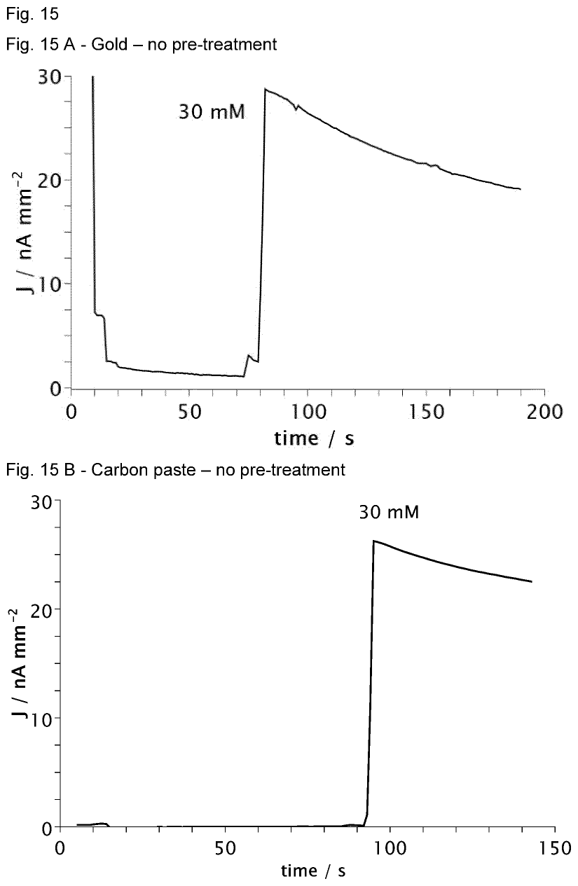

KmFCb2 immobilized on gold or carbon paste electrodes yields clear catalytic currents when adding 30 mM lactate without the need of a promotor layer as e. g. a thiol based self-assembled monolayer; see FIG. 15. Currents originate from direct electron transfer as no mediator is present.

### Example 8: Temperature Stability

The stability of different FCb2 enzymes was determined for the temperature range: 30° C., 34° C., 39° C., 41° C., 47° C., 51° C., 55° C., and 60° C. For incubation of the FCb2 enzymes, a thermocycler was used. The respective enzyme sample was incubated at a concentration of ˜0.4-1 mg/mL in 100 mM PPB, 1 mM EDTA, pH 7.0. Thereby, 70 μL aliquots were incubated at each time point (for triplicate activity measurement) and 70 μL were stored on ice for reference measurement. The samples were incubated for 30 min. After incubation, the samples were put on ice for 10 min. The remaining activities were assayed with CytC in PBS buffer, pH 7.4, at 30° C., 550 nm for 5 min. Thereafter, the T50 was determined. The T50 temperature or T50% temperature reflects the temperature at which an enzyme's residual activity is reduced by 50% after a heat challenge over a defined time. The T50 for KmFCb2 and CangFCb2 is shown in the following Table 3.

TABLE 3

| | T 50° C. |
|---|---|
| KmFCb2 | 40.9° C. |
| CangFCb2 | 40.9° C. |

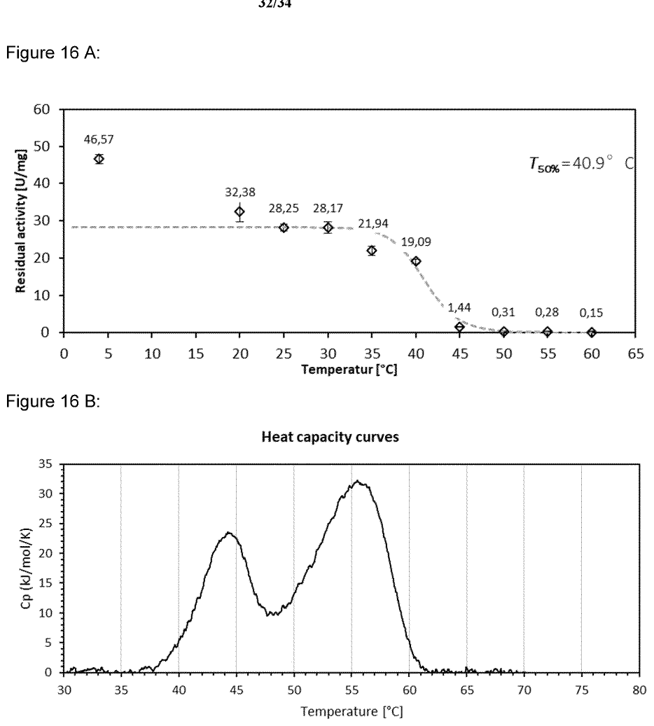

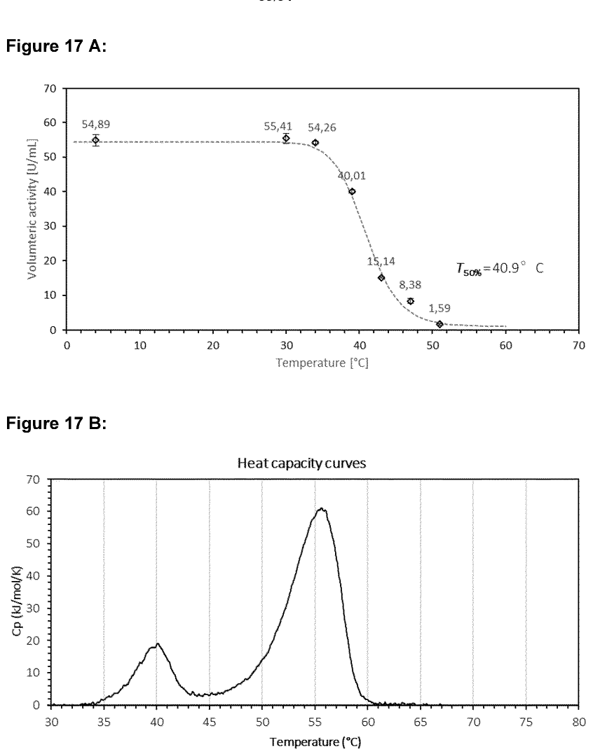

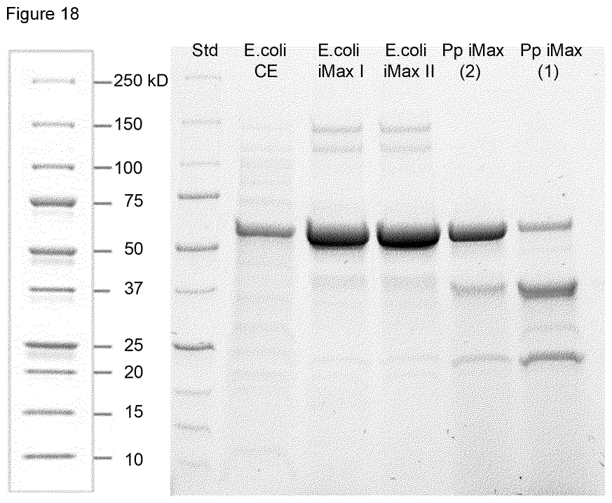

The data showing the remaining activity over the measured temperature range are show in FIG. 16 A for KmFCb2 (A) and FIG. 17 A for CangFCb2 (A). The heat capacity curve of KmFCb2 is shown in FIG. 16 B; the heat capacity curve of CangFCb2 is shown in FIG. 17 B. Heat capacity was determined by differential scanning calorimetry (DSC). Differential Scanning Calorimetry (DSC) measures heat consumption over a temperature gradient which allows to test the temperature dependent structural transitions of a sample and thus stability. Typically, data are presented as a melting temperature (Tm) and are an indication for thermal stability of a protein. A purified enzyme sample was used at 1.0 mg/mL in PBS buffer pH 7.4. The measurement was performed with PEAQ DSC Automated, Malvern Panalytical machine with respect to the same PBS buffer in the reference cell. Two scans of each sample have been done. The first scan gives the heat consumption data. Data from the second scan (rescan) can be used as blank in the data evaluation, if no peaks are determined, indicating fully denatured enzyme from the first scan. A temperature ramp at 20-100° C. with an increase of in 1° C./min steps (60° C./hr) was performed. And the same rate of change in temperature for cooling and the re-scan was used. Data processing was done with PEAQ Microcal software, using the rescan as a background and non-two-state fitting for the determination of melting temperatures.

The melting temperatures (Tm₁ and Tm₂) for KmFCb2 and CangFCb2 are shown in the following Table 4.

TABLE 4

| | Tm₁ | Tm₁ | Tm₂ | Tm₂ Error | TOnset |
|---|---|---|---|---|---|
| Sample | (° C.) | Error (° C.) | (° C.) | (° C.) | (° C.) |
| KmFCb2 | 44.26 | ±0.017 | 55.02 | ±0.012 | 36.69 |
| CangFCb2 | 39.80 | ±0.037 | 54.95 | ±0.020 | 34.67 |

### Example 9: Expression of FCb2 in Escherichia coli

In comparison to the expression of FCb2 in yeast cells, the expression of FCb2 was tested in E. coli BL21 cells and an expression trial of pET21 His8 KmFCb2 is shown in this example. Thereby, pET21 His8 KmFCb2 was transformed in E. coli BL21 expression strain. Cyt C activity of 8 transformed colonies was confirmed with a producer screening in deep well plate format. A fresh streak of the well showing the highest activity (8 U/mL) was prepared to get a single colony for the expression trial. Here, His KmFCb2 was expressed in E. coli BL21 in a scale of 4×250 mL TB_{Amp} medium. Cell pellets were harvested and stored at −20° C. for further purification trials.

For the pre-culture (overnight culture), 2×6 mL TB_{Amp} medium were inoculated with the same colony of E. coli BL21 pET21 His8 KmFCb2. The incubation was performed at 37° C., shaking, and overnight.

The main culture (fermentation) was performed at a scale of 4×250 mL TB_{Amp} medium. 250 mL TB_{Amp} medium were filled in 1 L Erlenmeyer flasks (without baffles, sterile). The medium was inoculated with 2.5 mL overnight culture and incubated at 37° C. and 150 rpm.

After 1 h it was started to check the OD600 until they reach a minimum of 0.5. The cultures were induced with 50 μL IPTG (500 mM) and incubated further at 20° C. and 250 rpm until the next day (Duration of expression circa 20 h).

### OD₆₀₀:

1 h incubation: Flask 1: 0.06; Flask 2: 0.05; Flask 3: 0.06; Flask 4: 0.06
2 h incubation: Flask 1: 0.18; Flask 2: 0.20; Flask 3: 0.19; Flask 4: 0.19
2 h 40 min incubation: Flask 1: 0.51; Flask 2: 0.53; Flask 3: 0.52; Flask 4: 0.54

Cell harvest: the cultures of each flask were transferred into a precooled 500 mL centrifuge beaker and centrifuged at 5000 rpm for 20 min at 4° C. The pellets were resuspended in 50 mM KPP (pH 6.5) and centrifuged again. The pellet was transferred to a tared falcon tube and washed two more times with buffer.
Weight of cell pellets: 1: 2.5 g; 2: 2.4 g; 3: 3.1 g; 4: 2.7 g

The pellets were stored at −30° C.

As a result, 4× pink colored cell pellets of 2.5 g to 3.1 g were harvested.

Concludingly, His8 KmFCb2 was expressed in E. coli BL21. 4× pink colored cell pellets of 2.5 to 3.1 g were harvested and stored at −20° C. for purification.

In the following, the IMAC purification trial of His8 KmFCb2 expressed in E. coli BL21 is described and the obtained specific activities are compared to activities from KmFCb2 expressed in yeast, more specifically in Pichia pastoris.

Cell disruption of E. coli: 2× frozen cell pellet of 2.4 and 3.1 g were thawed on ice and resuspended in Buffer A (filled to 10 mL). Right before cell disruption, 2000 μL PMSF (10 mg/mL) were added to the cell suspension. The cell suspension was homogenized in the French press in 4 consecutive cycles until liquids were freely flowing and not viscos anymore. A low temperature was maintained throughout the process.

After cell disruption, 30 mL lysate were carefully transferred into 50 mL centrifugation beakers. An additional 10 mL Buffer A was used to rinse the tube and transferred to the centrifugation beaker. The volume was brought to 40 mL with buffer A. Centrifugation was carried out at 20 000 rpm (48 000 g) at 4° C. for 30 minutes. After centrifugation, the supernatant was filled to 100 mL with Buffer A and filtered.

IMAC purification was performed using a 5 mL column. Different binding and elution buffers were used:
Buffer A: 50 mM PPB (Potassium), 500 mM NaCl, 50 mM Imidazol, pH 6.5
Buffer B: 50 mM PPB (Potassium), 500 mM NaCl, 750 mM Imidazol, pH 6.5

The purified enzyme was rebuffered to 100 mM PPB, 1 mM EDTA, pH 7.0.

The purification table of E. coli His8 KmFCb2 is given in the following table 5.

TABLE 5

| | | | 412/ | protein | total | cyt c | | | |
|---|---|---|---|---|---|---|---|---|---|
| | | Volume | 280 | conc. | protein | | | total | Yield |
| Step | Sample code | [mL] | RZ | [mg/mL] | [mg] | [U/mL] | [U/mg] | U | [%] |
| Sterile filtered | CE | 89 | 0.033 | 30.48 | 2713 | 2.97 | 0.0973 | 264 | 100% |
| Crude Extract | | | | | | | | | |
| Flow-through | FT_load | 89 | 0.023 | 21.53 | 1916 | 1.11 | 0.0516 | 99 | 37% |
| during load | | | | | | | | | |
| Flow-through | FT_wash | 50 | 0.024 | 5.232 | 261.6 | 0.320 | 0.0612 | 16 | 6% |
| during wash | | | | | | | | | |
| Pool elution | Pool I | 9.5 | 0.24 | 4.869 | 46.25 | 3.46 | 0.710 | 33 | 12% |
| F3-F10 | | | | | | | | | |
| Pool elution | Pool II | 10 | 0.21 | 4.950 | 49.50 | 2.04 | 0.413 | 20 | 8% |
| F11-F18 | | | | | | | | | |
| rebuffered | E. coli | 2.4 | 0.21 | 20.33 | 48.79 | 46.1 | 2.27 | 111 | 42% |
| and conc. | KmFCb2 I | | | | | | | | |
| Pool I | | | | | | | | | |
| rebuffered | E. coli | 2.7 | 0.19 | 16.31 | 44.04 | 34.5 | 2.11 | 93 | 35% |
| and conc. | KmFCb2 II | | | | | | | | |
| Pool II | | | | | | | | | |

The specific activities and RZ values E. coli vs P. pastoris expressed enzyme:
E. coli His8 KmFCb2: 2.2 U/mg; RZ: 0.2
P. pastoris His8 KmFCb2: 180 U/mg; RZ: 1.5

An SDS PAGE analysis was performed for E. coli CE, E. coli KmFCb2 final I, E. coli KmFCb2 final II, P. pastoris His8 KmFCb2, P. pastoris His8 KmFCb2.

The samples for the SDS PAGE were prepared by:
Purified enzyme samples are diluted to ˜1 mg/mL with H2O
E. coli CE is diluted to ˜3 mg/mL with H2O
15 μL sample are mixed with 15 μL Laemmli buffer (2×conc)
25 μL sample, 5.5 μL Protein Std are loaded on the gel
Gel is run at 90 V for 5 min, then at 150 V for 50 min

The result of the SDS PAGE is shown in FIG. 18. The expression of FCb2 in E. coli BL21 gives a thick band at around 58 kDa in E. coli CE. Purified His8 KmFCb2 of E. coli and P. pastoris shows same pattern of bands:
Band at ˜58 kDa indicates FCb2 (57.8 kDa theor. size)
Bands at ˜37 kDa and 25 kDa become more intense the longer the enzyme is stored at 4° C.

However, the purified FCb2 expressed in E. coli showed a distinctly less red colour than the purified FCb2 expressed in Pichia pastoris.

Concludingly, His8 KmFCb2 from E. coli was fully expressed in E. coli BL21 and could be purified via IMAC. Two purified enzyme samples: each around 45 mg; 100 U (40% yield). The specific activity was 2.2 U/mg and the RZ: 0.2. SDS PAGE confirmed the correct size of the enzyme (around 58 kDa). However, although E. coli His8 KmFCb2 had the correct size but showed low RZ values, a low specific activity with Cyt C and the color of the enzyme solution indicates fully expressed enzyme without heme.

Furthermore, the terminal electron transfer abilities in E. coli vs. P. pastoris have been compared using DCIP and CytC as electron acceptor. The following table 6 shows this comparison of CytC and DCIP activities in KmFCb2 from different expressions.

TABLE 6

| | DCIP | CytC | | RZ |
|---|---|---|---|---|
| | activity | activity | CytC activity/ | A412/ |
| | [U/mg] | [U/mg] | DCIP activity | A280 |
| KmFCb2 | 1.283 | 89.72 | 69.9 | 1.43 |
| E. coli KmFCb2 I | 0.948 | 1.09 | 1.1 | 0.21 |
| E. coli KmFCb2 II | 1.015 | 1.14 | 1.1 | 0.19 |

KmFCb2 was produced intracellularly in a truncated format (MHHHHHHHH-QNATKEELN: SEQ ID NO: 189) in P. pastoris with an N-terminal His8-tag as is described. It was isolated from the culture by mechanical cell disruption and purified via IMAC chromatography to apparent homogeneity. E. coli KmFCb2 was identical in the design and expressed intracellularly in E. coli, also relying in a N-terminal His8-tag. It was similarly released from the cell pellet by mechanical cell disruption (French press) and isolated via subsequent IMAC chromatography, where it eluted in two partially overlapping peaks (I and II). A drastically reduced spectral ratio and substantial loss in CytC activity, shift in CytC:DCIP ratio is clear evidence for a lack of the heme-cofactor due to the change in expression host. This is also observable from pictures of the purified enzyme solutions. The remaining minimal activity with CytC could point towards a small portion of holo-enzyme in the sample that incorporated the heme (E. coli b-type-heme synthesis are limited but not entirely absent). Alternatively, it could be a result of slow and unspecific interaction of the FMN DH moiety with the electrode (FMN diffusion). Interestingly, the DCIP activity is comparable between the three preparations and seems to no depend significantly on the expression host. In subsequent electrode measurements, no direct electron currents could be recorded for either of the E. coli produced KmFCb2 samples.

### REFERENCES

Abe, Yoshito, et al. 2000. “Structural Basis of Presequence Recognition by the Mitochondrial Protein Import Receptor Tom20.” Cell 100 (5): 551-60. https://doi.org/10.1016/S0092-8674(00)80691-1.
Beasley, E. M, et al. 1993. The signal that sorts yeast cytochrome b2 to the mitochondrial intermembrane space contains three distinct functional regions. The EMBO journal, 12(6), 2303-2311.
Black, M. T., et al. 1989. “Structural Basis for the Kinetic Differences between Flavocytochromes B2 from the Yeasts Hansenula Anomala and Saccharomyces Cerevisiae.” The Biochemical Journal 263 (3): 973-76. https://doi.org/10.1042/bj2630973.
Campo M. L., et al. 2017. Epub 2016 May 5. Revisiting trends on mitochondrial mega-channels for the import of proteins and nucleic acids. Journal of Bioenergetics and Biomembranes, Vol. 49(1), pp. 75-99
Diêp Lê, K. H., et al. 2009. “Interdomain Contacts in Flavocytochrome B2, a Mutational Analysis.” Biochemistry 48 (45): 10803-9. https://doi.org/10.1021/bi901301r.
Diekert, K., et al. (2001). Apocytochrome c requires the TOM complex for translocation across the mitochondrial outer membrane. The EMBO journal, 20(20), 5626-5635.
Edwards, R., et al. 2021. The mitochondrial intermembrane space: the most constricted mitochondrial sub-compartment with the largest variety of protein import pathways. Open biology, 11(3), 210002. https://doi.org/10.1098/rsob.210002
Gakh, Oleksandr, et al. 2002. “Mitochondrial Processing Peptidases.” Biochimica et Biophysica Acta—Molecular Cell Research. Biochim Biophys Acta. https://doi.org/10.1016/S0167-4889(02)00265-3.
Gaume, B., et al. 1995. “Mutation to Glutamine of Histidine 373, the Catalytic Base of Flavocytochrome B2 (L-Lactate Dehydrogenase).” Biochimie 77 (7-8): 621-30. https://doi.org/10.1016/0300-9084(96)88177-6.
Geiss, A. F., et al. (2021). Engineering the Turnover Stability of Cellobiose Dehydrogenase toward Long-Term Bioelectronic Applications. ACS sustainable chemistry & engineering, 9(20), 7086-7100. https://doi.org/10.1021/acssuschemeng.1c01165
Ghrir, Rachid, et al. 1984. “Primary Structure of Flavocytochrome B2 from Baker's Yeast: Purification by Reverse-phase High-pressure Liquid Chromatography and Sequencing of Fragment a Cyanogen Bromide Peptides.” European Journal of Biochemistry 139 (1): 59-74. https://doi.org/10.1111/j.1432-1033.1984.tb07976.x.
Grandier-Vazeille, X., et al. 2001. “Yeast Mitochondrial Dehydrogenases Are Associated in a Supramolecular Complex.” Biochemistry 40 (33): 9758-69. https://doi.org/10.1021/bi010277r.
Haumont, Pierre-et al. 1987. “Amino-acid Sequence of the Cytochrome-b5-like Heme-binding Domain from Hansenula Anomala Flavocytochrome B2.” European Journal of Biochemistry 169 (3): 539-44. https://doi.org/10.1111/j.1432-1033.1987. tb13642. x.
Hartl F. U. et al. 1987. Successive translocation into and out of the mitochondrial matrix: Targeting of proteins to the intermembrane space by a bipartite signal peptide. CELL, ELSEVIER Vol. 51(6), pp. 1027-1037
Hiraka, Kentaro, et al. 2020. “Rational Engineering of Aerococcus Viridans L-Lactate Oxidase for the Mediator Modification to Achieve Quasi-Direct Electron Transfer Type Lactate Sensor.” Biosensors and Bioelectronics 151 (March). https://doi.org/10.1016/j.bios.2019.111974.
Kaushik, Neha, et al. 2020. “Enhanced Cell Density Cultivation and Rapid Expression-Screening of Recombinant Pichia Pastoris Clones in Microscale.” Scientific Reports 10 (1). https://doi.org/10.1038/s41598-020-63995-5.
Kavita R. et al. 2016. Biosensors based on electrochemical lactate detection: A comprehensive review. Biochemistry and Biophysics Reports, Vol. 5, pp. 35-54
Koll H., et al. 1992. Antifolding activity of hsp60 couples protein import into the mitochondrial matrix with export to the intermembrane space, Cell, Volume 68, Issue 6, Pages 1163-1175
Lederer, Florence, et al. 1985. “Complete Amino Acid Sequence of Flavocytochrome B2 from Baker's Yeast.” European Journal of Biochemistry 152 (2): 419-28. https://doi.org/10.1111/j.1432-1033.1985.tb09213.x.
Petrović, Dejan M., et al. “Methylation of the N-terminal histidine protects a lytic polysaccharide monooxygenase from auto-oxidative inactivation.” Protein Science 27.9 (2018): 1636-1650.
Smutok, Oleh, et al. 2013. “Flavocytochrome b 2-Based Enzymatic Method of L-Lactate Assay in Food Products.” The Scientific World Journal 2013: 1-6. https://doi.org/10.1155/2013/461284.
Vögtle, F. et al. 2012. “Intermembrane Space Proteome of Yeast Mitochondria.” Molecular and Cellular Proteomics 11 (12): 1840-52. https://doi.org/10.1074/mcp.M112.021105.
Xia, Zong Xiang, and F. Scott Mathews. 1990. “Molecular Structure of Flavocytochrome B2 at 24 Å Resolution.” Journal of Molecular Biology 212 (4): 837-63. https://doi.org/10.1016/0022-2836(90)90240-M.

1. A recombinant flavocytochrome b2 (FCb2), comprising a mature FCb2 peptide sequence of a native FCb2, or a functionally active variant of said mature FCb2 peptide sequence, wherein the N-terminus of said mature FCb2 peptide sequence consists of a truncated signal peptide sequence replacing the native signal peptide sequence, and wherein said truncated signal peptide sequence is of the following structure from N- to C-terminus:
i. a methionine;
ii. optionally a tag sequence;
iii. an amino acid sequence of a length of 0, 1, 2, 3, 4, 5, 6, 7, 8, 9 amino acids of the native signal peptide sequence, corresponding to the amino acid sequence up to 9 amino acids directly upstream of the I/L/V-x-N/A/L motif of the native signal peptide sequence of FCb2; and
iv. the I/L/V-x-N/A/L motif, which can be of number 0, 1, or can be partially truncated, wherein if the number is 0 or the I/L/V-x-N/A/L is partially truncated, the length of the amino acid sequence of iii. is 0,
wherein the amino acid sequence of said recombinant FCb2 is different from SEQ ID NO: 145.

2. The recombinant FCb2 of claim 1, wherein
i. the mature FCb2 peptide sequence is derived from Saccharomyces cerevisiae FCb2 and the truncated signal peptide sequence is selected from the group consisting of SEQ ID NOs:37, 38 and 39; or
ii. wherein the mature FCb2 peptide sequence is derived from Kluyveromyces marxianus FCb2 and the truncated signal peptide sequence is selected from the group consisting of SEQ ID NOs:40, 41 and 42; or
iii. wherein the mature FCb2 peptide sequence is derived from Wickerhamomyces anomalus FCb2 and the truncated signal peptide sequence is SEQ ID NO:43 or 44 or it is of the amino acid sequence M S A.

3. The recombinant FCb2 of claim 1 or 2, wherein the tag sequence is selected from the group consisting of affinity tags, solubility enhancement tags, and monitoring tags.

4. The recombinant FCb2 of any one of claims 1 to 3, having an amino acid sequence selected from SEQ ID NOs:4, 5, 6, 8, 9, 10, 12, 13, 14, 28, 29, 30, 31, 32, 33, 34, 35, and 36.

5. An electrode comprising a recombinant flavocytochrome b2 (FCb2) comprising a mature FCb2 peptide sequence of a native FCb2, or a functionally active variant of said mature FCb2 peptide sequence, with an N-terminus consisting of a truncated signal peptide sequence replacing the native signal peptide sequence, wherein said truncated signal peptide sequence is of the following structure from N- to C-terminus:
i. a methionine;
ii. optionally a tag sequence;
iii. an amino acid sequence of a length of 0, 1, 2, 3, 4, 5, 6, 7, 8, 9 amino acids of the native signal peptide sequence, corresponding to the amino acid sequence up to 9 amino acids directly upstream of the I/L/V-x-N/A/L motif of the native signal peptide sequence of FCb2; and
iv. the I/L/V-x-N/A/L motif, which can be of number 0, 1, or can be partially truncated, wherein if the number is 0 or the I/L/V-x-N/A/L is partially truncated, the length of the amino acid sequence of iii. is 0.

6. A method of producing a recombinant flavocytochrome b2 (FCb2), comprising the steps of:
i. providing a yeast cell comprising a promoter functional in yeast operably linked to a recombinant nucleic acid sequence encoding an FCb2, wherein the native signal peptide sequence of said FCb2 has been replaced with a truncated signal peptide sequence consisting of the following structure from N- to C-terminus:
a) a methionine;
b) optionally a tag sequence;
c) an amino acid sequence of a length of 0, 1, 2, 3, 4, 5, 6, 7, 8, 9 amino acids of the native signal peptide sequence, corresponding to the amino acid sequence up to 9 amino acids directly upstream of the I/L/V-x-N/A/L motif of the native signal peptide sequence of FCb2; and
d) the I/L/V-x-N/A/L motif, which can be of number 0, 1, or can be partially truncated, wherein if the number is 0 or the I/L/V-x-N/A/L is partially truncated, the length of the amino acid sequence of c) is 0; and
ii. cultivating said yeast cell in a medium conducive for the expression of said FCb2.

7. The method of claim 6, wherein the recombinant nucleic acid sequence encoding said FCb2 is stably integrated into the yeast cell's genome.

8. The method of claim 6 or 7, wherein the yeast cells are methylotrophic yeast cells, preferably Pichia pastoris cells, or non-methylotrophic yeast cells, preferably Saccharomyces cerevisiae cells.

9. The method of any one of claims 6 to 8, wherein the nucleic acid sequence encoding the FCb2 is derived from S. cerevisiae, W anomalus, K. marxianus, or O. parapolymorpha.

10. The method of claim 9, wherein
i. the mature FCb2 peptide sequence is derived from S. cerevisiae FCb2 and the truncated signal peptide sequence is selected from the group consisting of SEQ ID NOs:37, 38, and 39; or
ii. wherein the mature FCb2 peptide sequence is derived from K. marxianus FCb2 and the truncated signal peptide sequence is selected from the group consisting of SEQ ID NOs:40, 41, and 42; or
iii. wherein the mature FCb2 peptide sequence is derived from W. anomalus FCb2 and the truncated signal peptide sequence is SEQ ID NO:43 or 44 or it is of the amino acid sequence M S A.

11. The method of any one of claims 6 to 10, wherein the recombinant FCb2 comprises an N-terminal tag sequence, preferably selected from the group consisting of affinity tags, solubility enhancement tags and monitoring tags, and most preferably a His tag.

12. The method of any one of claims 6 to 11, wherein the recombinant FCb2 has a sequence selected from the group consisting of SEQ ID NOs:4, 5, 6, 8, 9, 10, 12, 13, 14, 33, 34, 35, and 36.

13. The method of any one of claims 6 to 12, further comprising the step of isolating the FCb2 from the yeast cell, preferably by disrupting the yeast cells to release the intracellular FCb2 into the medium followed by purifying the FCb2 from the medium.

14. A nucleic acid molecule encoding the recombinant FCb2 of any one of claims 1 to 4.

15. A yeast host cell comprising a nucleic acid molecule encoding a mature FCb2 peptide sequence of a native FCb2, or a functionally active variant of said mature FCb2 peptide sequence, with an N-terminus consisting of a truncated signal peptide sequence replacing the native signal peptide sequence, wherein said truncated signal peptide sequence is of the following structure from N- to C-terminus:
i. a methionine;
ii. optionally a tag sequence;
iii. an amino acid sequence of a length of 0, 1, 2, 3, 4, 5, 6, 7, 8, 9 amino acids of the native signal peptide sequence, corresponding to the amino acid sequence up to 9 amino acids directly upstream of the I/L/V-x-N/A/L motif of the native signal peptide sequence of FCb2; and
iv. the I/L/V-x-N/A/L motif, which can be of number 0, 1, or can be partially truncated, wherein if the number is 0 or the I/L/V-x-N/A/L is partially truncated, the length of the amino acid sequence of iii. is 0.


SEQUENCE LISTING (189 sequences)

SEQ ID NO: 1 - protein, 511 aa, Saccharomyces cerevisiae
EPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 2 - protein, 500 aa, Wickerhamomyces anomalus
DVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 3 - protein, 591 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 4 - protein, 521 aa, Artificial Sequence
  Polypeptide
MNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 5 - protein, 516 aa, Artificial Sequence
  Polypeptide
MQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 6 - protein, 512 aa, Artificial Sequence
  Polypeptide
MEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 7 - protein, 592 aa, Kluyveromyces marxianus
MRSAARVINKSCSGSALSRRCLRKSSLSMSMRYLSTSNIGVRKGFNGQGKSSNKTMLFLAAGASAVAGIGLLSQFSDSLQNATKEELNKPKVSPLEVAKHSSPDDCWVVIDGFVYNLTEFISAHPGGPAIIENNAGKDVTAIFGPIHAPDVIEKYIAPENRIGPLDGKMPDDLICAPLTPGETPEDVARKEELRQNMPDLDSLVNIYDFEFLASQILTKQAWSYYSSAADDEVTHRENHAAYHRIFFKPRILVNVKEVDTSTTMLGEKVGVPFYVSATALCKLGNPKEGEKDIARGCGESDVKPIQMISTLASCSLQEIVEAAPSKDQIQWFQLYVNSDRKITEELIKNVEKLGLKAIFVTVDAPSLGNREKDAKVKFTNKDSSAKAMEKSNVKESKGASRALSTFIDPALCWDDIVTLKSKTKLPIVIKGVQCVEDVLKAAEIGAAGVVLSNHGGRQLDFSRAPIEVLAETMPILKEKKLDDKIEIFIDGGVRRGTDILKALCLGAKGVGLGRPFLYANSCYGKEGVKKAIELLKDELEMSMRLLGVTSIDQLSEKYLDLSTLHGRTVSVPRDNLYNGVYVPHEPTDFKEN

SEQ ID NO: 8 - protein, 522 aa, Artificial Sequence
  Polypeptide
MLSQFSDSLQNATKEELNKPKVSPLEVAKHSSPDDCWVVIDGFVYNLTEFISAHPGGPAIIENNAGKDVTAIFGPIHAPDVIEKYIAPENRIGPLDGKMPDDLICAPLTPGETPEDVARKEELRQNMPDLDSLVNIYDFEFLASQILTKQAWSYYSSAADDEVTHRENHAAYHRIFFKPRILVNVKEVDTSTTMLGEKVGVPFYVSATALCKLGNPKEGEKDIARGCGESDVKPIQMISTLASCSLQEIVEAAPSKDQIQWFQLYVNSDRKITEELIKNVEKLGLKAIFVTVDAPSLGNREKDAKVKFTNKDSSAKAMEKSNVKESKGASRALSTFIDPALCWDDIVTLKSKTKLPIVIKGVQCVEDVLKAAEIGAAGVVLSNHGGRQLDFSRAPIEVLAETMPILKEKKLDDKIEIFIDGGVRRGTDILKALCLGAKGVGLGRPFLYANSCYGKEGVKKAIELLKDELEMSMRLLGVTSIDQLSEKYLDLSTLHGRTVSVPRDNLYNGVYVPHEPTDFKEN

SEQ ID NO: 9 - protein, 517 aa, Artificial Sequence
  Polypeptide
MDSLQNATKEELNKPKVSPLEVAKHSSPDDCWVVIDGFVYNLTEFISAHPGGPAIIENNAGKDVTAIFGPIHAPDVIEKYIAPENRIGPLDGKMPDDLICAPLTPGETPEDVARKEELRQNMPDLDSLVNIYDFEFLASQILTKQAWSYYSSAADDEVTHRENHAAYHRIFFKPRILVNVKEVDTSTTMLGEKVGVPFYVSATALCKLGNPKEGEKDIARGCGESDVKPIQMISTLASCSLQEIVEAAPSKDQIQWFQLYVNSDRKITEELIKNVEKLGLKAIFVTVDAPSLGNREKDAKVKFTNKDSSAKAMEKSNVKESKGASRALSTFIDPALCWDDIVTLKSKTKLPIVIKGVQCVEDVLKAAEIGAAGVVLSNHGGRQLDFSRAPIEVLAETMPILKEKKLDDKIEIFIDGGVRRGTDILKALCLGAKGVGLGRPFLYANSCYGKEGVKKAIELLKDELEMSMRLLGVTSIDQLSEKYLDLSTLHGRTVSVPRDNLYNGVYVPHEPTDFKEN

SEQ ID NO: 10 - protein, 512 aa, Artificial Sequence
  Polypeptide
MATKEELNKPKVSPLEVAKHSSPDDCWVVIDGFVYNLTEFISAHPGGPAIIENNAGKDVTAIFGPIHAPDVIEKYIAPENRIGPLDGKMPDDLICAPLTPGETPEDVARKEELRQNMPDLDSLVNIYDFEFLASQILTKQAWSYYSSAADDEVTHRENHAAYHRIFFKPRILVNVKEVDTSTTMLGEKVGVPFYVSATALCKLGNPKEGEKDIARGCGESDVKPIQMISTLASCSLQEIVEAAPSKDQIQWFQLYVNSDRKITEELIKNVEKLGLKAIFVTVDAPSLGNREKDAKVKFTNKDSSAKAMEKSNVKESKGASRALSTFIDPALCWDDIVTLKSKTKLPIVIKGVQCVEDVLKAAEIGAAGVVLSNHGGRQLDFSRAPIEVLAETMPILKEKKLDDKIEIFIDGGVRRGTDILKALCLGAKGVGLGRPFLYANSCYGKEGVKKAIELLKDELEMSMRLLGVTSIDQLSEKYLDLSTLHGRTVSVPRDNLYNGVYVPHEPTDFKEN

SEQ ID NO: 11 - protein, 573 aa, Wickerhamomyces anomalus
MFKSQLRTATARSSFRSLASKLNPQRFNSSKTPLLNATRGSNRSKNSLIALAISLSAVSSSYYLYQKDKFISADVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 12 - protein, 513 aa, Artificial Sequence
  Polypeptide
MYYLYQKDKFISADVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 13 - protein, 508 aa, Artificial Sequence
  Polypeptide
MKDKFISADVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 14 - protein, 503 aa, Artificial Sequence
  Polypeptide
MSADVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 15 - protein, 80 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDN

SEQ ID NO: 16 - protein, 593 aa, Candida glabrata
MQRYIFKTPHSRVLRSLPRHAINSTTKRGIVSSRSSLNNQFFNSERQRKSYKWFWSIGLTVGLTAAIADNLHNKKDIYNDAKFDSSKPKISPSEVIKHNTPEDCWVVIDGYVYDLTNFIALHPGGPDIIKTNAGKDVTAIFDPIHPPDAIEKYIKPEQHVGPLDGKLDAEYICPPYAPGETPDDIARKAALRARLPPLSSIMNLYDFEYLASQILSKQAWAYYSSASDDEVSYRENHNAYHRIFFNPKVLVDVSKVDTSTEMLGHKVDVPFYVTATALCKLGNPKEGEKDIARGCGQGPNKTPQMISTLASCSVDEIVNAAPSKDQVIWYQLYVNSDRKITENLIKHVEDLGVKAIFVTVDAPSLGSREKDKKVKFNNTMSGPKSMKKSDVGESEGAAQTLSKFIDPSLSWQDIKILRKKTKLPIVIKGVQRVQDVVKAAEIGCNGVVLSNHGGRQLDFARAPIEVLAETMPVLKEKKLDKNFEVFVDGGVRRGTDVIKALCLGASGVGLGRPFLYANSCYGKDGVQKAIDLLKTEIEMNMRLLGVTSIKDMNPELLDLSSLHGRTVNVPKDSLYVNVYNKPELAEFLDDASD

SEQ ID NO: 17 - protein, 599 aa, Artificial Sequence
  Polypeptide
MHHHHHHHHLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPRILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 18 - protein, 600 aa, Artificial Sequence
  Polypeptide
MHHHHHHHHRSAARVINKSCSGSALSRRCLRKSSLSMSMRYLSTSNIGVRKGFNGQGKSSNKTMLFLAAGASAVAGIGLLSQFSDSLQNATKEELNKPKVSPLEVAKHSSPDDCWVVIDGFVYNLTEFISAHPGGPAIIENNAGKDVTAIFGPIHAPDVIEKYIAPENRIGPLDGKMPDDLICAPLTPGETPEDVARKEELRQNMPDLDSLVNIYDFEFLASQILTKQAWSYYSSAADDEVTHRENHAAYHRIFFKPRILVNVKEVDTSTTMLGEKVGVPFYVSATALCKLGNPKEGEKDIARGCGESDVKPIQMISTLASCSLQEIVEAAPSKDQIQWFQLYVNSDRKITEELIKNVEKLGLKAIFVTVDAPSLGNREKDAKVKFTNKDSSAKAMEKSNVKESKGASRALSTFIDPALCWDDIVTLKSKTKLPIVIKGVQCVEDVLKAAEIGAAGVVLSNHGGRQLDFSRAPIEVLAETMPILKEKKLDDKIEIFIDGGVRRGTDILKALCLGAKGVGLGRPFLYANSCYGKEGVKKAIELLKDELEMSMRLLGVTSIDQLSEKYLDLSTLHGRTVSVPRDNLYNGVYVPHEPTDFKEN

SEQ ID NO: 19 - protein, 581 aa, Artificial Sequence
  Polypeptide
MHHHHHHHHFKSQLRTATARSSFRSLASKLNPQRFNSSKTPLLNATRGSNRSKNSLIALAISLSAVSSSYYLYQKDKFISADVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 20 - protein, 601 aa, Artificial Sequence
  Polypeptide
MHHHHHHHHQRYIFKTPHSRVLRSLPRHAINSTTKRGIVSTRSSLNNQFFNSERQRKSYKWFWSIGLTVGLTAAIADNLHNKKDIYNDAKFDSSKPKISPSEVIKHNTPEDCWVVIDGYVYDLTNFIALHPGGPDIIKTNAGKDVTAIFDPIHPPDAIEKYIKPEQHVGPLDGKLDAEYICPPYAPGETPDDIARKAALRARLPPLSSIMNLYDFEYLASQILSKQAWAYYSSASDDEVSYRENHNAYHRIFFNPKVLVDVSKVDTSTEMLGHKVDVPFYVTATALCKLGNPKEGEKDIARGCGQGPNKTPQMISTLASCSVDEIVNAAPSKDQVIWYQLYVNSDRKITENLIKHVEDLGVKAIFVTVDAPSLGSREKDKKVKFNNTMSGPKSMKKSDVGESEGAAQTLSKFIDPSLSWQDIKILRKKTKLPIVIKGVQRVQDVVKAAEIGCNGVVLSNHGGRQLDFARAPIEVLAETMPVLKEKKLDKNFEVFVDGGVRRGTDVIKALCLGASGVGLGRPFLYANSCYGKDGVQKAIDLLKTEIEMNMRLLGVTSIKDMNPELLDLSSLHGRTVNVPKDSLYVNVYNKPELAEFLDDASD

SEQ ID NO: 21 - protein, 568 aa, Artificial Sequence
  Polypeptide
MHHHHHHHHGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPRILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 22 - protein, 568 aa, Artificial Sequence
  Polypeptide
MHHHHHHHHLSTSNIGVRKGFNGQGKSSNKTMLFLAAGASAVAGIGLLSQFSDSLQNATKEELNKPKVSPLEVAKHSSPDDCWVVIDGFVYNLTEFISAHPGGPAIIENNAGKDVTAIFGPIHAPDVIEKYIAPENRIGPLDGKMPDDLICAPLTPGETPEDVARKEELRQNMPDLDSLVNIYDFEFLASQILTKQAWSYYSSAADDEVTHRENHAAYHRIFFKPRILVNVKEVDTSTTMLGEKVGVPFYVSATALCKLGNPKEGEKDIARGCGESDVKPIQMISTLASCSLQEIVEAAPSKDQIQWFQLYVNSDRKITEELIKNVEKLGLKAIFVTVDAPSLGNREKDAKVKFTNKDSSAKAMEKSNVKESKGASRALSTFIDPALCWDDIVTLKSKTKLPIVIKGVQCVEDVLKAAEIGAAGVVLSNHGGRQLDFSRAPIEVLAETMPILKEKKLDDKIEIFIDGGVRRGTDILKALCLGAKGVGLGRPFLYANSCYGKEGVKKAIELLKDELEMSMRLLGVTSIDQLSEKYLDLSTLHGRTVSVPRDNLYNGVYVPHEPTDFKEN

SEQ ID NO: 23 - protein, 555 aa, Artificial Sequence
  Polypeptide
MHHHHHHHHNSSKTPLLNATRGSNRSKNSLIALAISLSAVSSSYYLYQKDKFISADVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 24 - protein, 520 aa, Artificial Sequence
  Polypeptide
MHHHHHHHHEPKLDMNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPRILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 25 - protein, 520 aa, Artificial Sequence
  Polypeptide
MHHHHHHHHATKEELNKPKVSPLEVAKHSSPDDCWVVIDGFVYNLTEFISAHPGGPAIIENNAGKDVTAIFGPIHAPDVIEKYIAPENRIGPLDGKMPDDLICAPLTPGETPEDVARKEELRQNMPDLDSLVNIYDFEFLASQILTKQAWSYYSSAADDEVTHRENHAAYHRIFFKPRILVNVKEVDTSTTMLGEKVGVPFYVSATALCKLGNPKEGEKDIARGCGESDVKPIQMISTLASCSLQEIVEAAPSKDQIQWFQLYVNSDRKITEELIKNVEKLGLKAIFVTVDAPSLGNREKDAKVKFTNKDSSAKAMEKSNVKESKGASRALSTFIDPALCWDDIVTLKSKTKLPIVIKGVQCVEDVLKAAEIGAAGVVLSNHGGRQLDFSRAPIEVLAETMPILKEKKLDDKIEIFIDGGVRRGTDILKALCLGAKGVGLGRPFLYANSCYGKEGVKKAIELLKDELEMSMRLLGVTSIDQLSEKYLDLSTLHGRTVSVPRDNLYNGVYVPHEPTDFKEN

SEQ ID NO: 26 - protein, 509 aa, Artificial Sequence
  Polypeptide
MHHHHHHHHDVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 27 - protein, 523 aa, Artificial Sequence
  Polypeptide
MHHHHHHHHDAKFDSSKPKISPSEVIKHNTPEDCWVVIDGYVYDLTNFIALHPGGPDIIKTNAGKDVTAIFDPIHPPDAIEKYIKPEQHVGPLDGKLDAEYICPPYAPGETPDDIARKAALRARLPPLSSIMNLYDFEYLASQILSKQAWAYYSSASDDEVSYRENHNAYHRIFFNPKVLVDVSKVDTSTEMLGHKVDVPFYVTATALCKLGNPKEGEKDIARGCGQGPNKTPQMISTLASCSVDEIVNAAPSKDQVIWYQLYVNSDRKITENLIKHVEDLGVKAIFVTVDAPSLGSREKDKKVKFNNTMSGPKSMKKSDVGESEGAAQTLSKFIDPSLSWQDIKILRKKTKLPIVIKGVQRVQDVVKAAEIGCNGVVLSNHGGRQLDFARAPIEVLAETMPVLKEKKLDKNFEVFVDGGVRRGTDVIKALCLGASGVGLGRPFLYANSCYGKDGVQKAIDLLKTEIEMNMRLLGVTSIKDMNPELLDLSSLHGRTVNVPKDSLYVNVYNKPELAEFLDDASD

SEQ ID NO: 28 - protein, 510 aa, Artificial Sequence
  Polypeptide
MKHHHHHHHHDVPHWKDIELTPEIVSQHNKKDDLWVVLNGQVYDLTDFLPNHPGGQKIIIRYAGKDATKIFVPIHPPDTIEKFIPPEKHLGPLVGEFEQEEEELSDEEIDRLERIERKPPLSQMINLHDFETIARQILPPPALAYYCSAADDEVTLRENHNAYHRIFFNPKILIDVKDVDISTEFFGEKTSAPFYISATALAKLGHPEGEVAIAKGAGREDVVQMISTLASCSFDEIADARIPGQQQWYQLYVNADRSITEKAVRHAEERGMKGLFITVDAPSLGRREKDMKMKFEADSDVQGDDEDIDRSQGASRALSSFIDPSLSWKDIAFIKSITKMPIVIKGVQRKEDVLLAAEHGLQGVVLSNHGGRQLDYTRAPVEVLAEVMPILKERGLDQKIDIFVDGGVRRGTDVLKALCLGAKGVGLGRPFLYAMSSYGDKGVTKAIQLLKDEIEMNMRLLGVNKIEELTPELLDTRSIHNRAVPVAKDYLYEQNYQRMSGAEFRPGIED

SEQ ID NO: 29 - protein, 523 aa, Artificial Sequence
  Polypeptide
MKHHHHHHHHQNATKEELNKPKVSPLEVAKHSSPDDCWVVIDGFVYNLTEFISAHPGGPAIIENNAGKDVTAIFGPIHAPDVIEKYIAPENRIGPLDGKMPDDLICAPLTPGETPEDVARKEELRQNMPDLDSLVNIYDFEFLASQILTKQAWSYYSSAADDEVTHRENHAAYHRIFFKPRILVNVKEVDTSTTMLGEKVGVPFYVSATALCKLGNPKEGEKDIARGCGESDVKPIQMISTLASCSLQEIVEAAPSKDQIQWFQLYVNSDRKITEELIKNVEKLGLKAIFVTVDAPSLGNREKDAKVKFTNKDSSAKAMEKSNVKESKGASRALSTFIDPALCWDDIVTLKSKTKLPIVIKGVQCVEDVLKAAEIGAAGVVLSNHGGRQLDFSRAPIEVLAETMPILKEKKLDDKIEIFIDGGVRRGTDILKALCLGAKGVGLGRPFLYANSCYGKEGVKKAIELLKDELEMSMRLLGVTSIDQLSEKYLDLSTLHGRTVSVPRDNLYNGVYVPHEPTDFKEN

SEQ ID NO: 30 - protein, 526 aa, Artificial Sequence
  Polypeptide
MKHHHHHHHHYNDAKFDSSKPKISPSEVIKHNTPEDCWVVIDGYVYDLTNFIALHPGGPDIIKTNAGKDVTAIFDPIHPPDAIEKYIKPEQHVGPLDGKLDAEYICPPYAPGETPDDIARKAALRARLPPLSSIMNLYDFEYLASQILSKQAWAYYSSASDDEVSYRENHNAYHRIFFNPKVLVDVSKVDTSTEMLGHKVDVPFYVTATALCKLGNPKEGEKDIARGCGQGPNKTPQMISTLASCSVDEIVNAAPSKDQVIWYQLYVNSDRKITENLIKHVEDLGVKAIFVTVDAPSLGSREKDKKVKFNNTMSGPKSMKKSDVGESEGAAQTLSKFIDPSLSWQDIKILRKKTKLPIVIKGVQRVQDVVKAAEIGCNGVVLSNHGGRQLDFARAPIEVLAETMPVLKEKKLDKNFEVFVDGGVRRGTDVIKALCLGASGVGLGRPFLYANSCYGKDGVQKAIDLLKTEIEMNMRLLGVTSIKDMNPELLDLSSLHGRTVNVPKDSLYVNVYNKPELAEFLDDASD

SEQ ID NO: 31 - protein, 512 aa, Artificial Sequence
  Polypeptide
MKHHHHHHHHIRLDAPTQGPLYTPDDVSAHNTKDDLWVVLNGKVYDLTEFLPNHPGGQRIILKYAGKDASKIFNPIHPPDAIDKYLPPEKCIGTLDGEFPEEEEVLSDEELLRLDRVDRKPPLSKIMNLHDFEAVARQILPPGALAYYSSAADDEITLRENHNAYHRIFFNPKVLVDVSKVDITTEFLGNETSAPFYITATALAKLGHPDGEVSIARGAGKEDITQMISTLASCSFDQIADARVKDTQAQWYQLYVSEDRELSKKQLKHCEERGMKGIFITVDAPSLGRREKDMKLKFEGNADVQSDDDIDRSQGAAVALSSFIDSSLTWKDITEIRKWTKLPIVIKGVQRKEDVLLALEHGCDGVVLSNHGGRQLDFSRAPVEVLAEVMPILREKGVDKKFEVYVDGGVRRGTDVLKALCLGAKGVGLGRPFLYANSAYGEKGVRRAIQLLKSELIMNMRLLGVTKIEDLTPELLDTTTIHQRTVSVPADYLYKENYLHMQGVEFNPELEE

SEQ ID NO: 32 - protein, 535 aa, Artificial Sequence
  Polypeptide
MKHHHHHHHHQNESKLAPKRVIDPSEVARHNTPADCWIVINGVVYDLTSFIPVHPGGADIIKSNAGKDVTAIFEPIHAPGVIEKYLPPKCRIGTLKKPMPAELTCDPYTPGESKDDIIKKLELRNNLPHLDSIINIYDFEKLASKILSNQAWAYYSSGADDEISYRDNHSAYRRIFFKPRILRDVSSVDVKTTMLGSKVDVPFYVSATALCKLGNPKEGEKDIARGCGQGDTKVPQMISTLASCSVDEIVDAAPSKDQIAWYQVYVNSDRNITRDMIKHVEKLGIKALFITVDAPSLGRREKDMKIKFSGSDQGAKVMKEPLKKVEKKDDGEMSKGASTTLSKFIDPSLTWDDVVKMRKWTKLPIVIKGVQSVEDVVKAAELGVDGVVLSNHGGRQLDYSRPPIEVLAETVPVLKEKHLDGKLELFVDGGVRRGTDVIKALCLGAKGVGLGRPFLYSNSCYGKDGVEKTIELLKTEIEMSMRLLGVTSIDQLTPELLDTTNLKSRTVNVSKDVLFDTVYRNPTLAAFQNGYDDDE

SEQ ID NO: 33 - protein, 520 aa, Artificial Sequence; identical to SEQ ID NO: 24
  Polypeptide

SEQ ID NO: 34 - protein, 520 aa, Artificial Sequence; identical to SEQ ID NO: 25
  Polypeptide

SEQ ID NO: 35 - protein, 509 aa, Artificial Sequence; identical to SEQ ID NO: 26
  Polypeptide

SEQ ID NO: 36 - protein, 523 aa, Artificial Sequence; identical to SEQ ID NO: 27
  Polypeptide

SEQ ID NO: 37 - protein, 15 aa, Artificial Sequence
  Polypeptide
MNWHNGQIDNEPKLD

SEQ ID NO: 38 - protein, 10 aa, Artificial Sequence
  Polypeptide
MQIDNEPKLD

SEQ ID NO: 39 - protein, 6 aa, Artificial Sequence
  Polypeptide
MEPKLD

SEQ ID NO: 40 - protein, 16 aa, Artificial Sequence
  Polypeptide
MLSQFSDSLQNATKEE

SEQ ID NO: 41 - protein, 11 aa, Artificial Sequence
  Polypeptide
MDSLQNATKEE

SEQ ID NO: 42 - protein, 6 aa, Artificial Sequence
  Polypeptide
MATKEE

SEQ ID NO: 43 - protein, 13 aa, Artificial Sequence
  Polypeptide
MYYLYQKDKFISA

SEQ ID NO: 44 - protein, 8 aa, Artificial Sequence
  Polypeptide
MKDKFISA

SEQ ID NO: 45 - protein, 5 aa, Saccharomyces cerevisiae
EPKLD

SEQ ID NO: 46 - protein, 85 aa, Artificial Sequence
  Polypeptide
MLKYKPLLKISKNCEAAILRASKTRLNTIRAYGSTVPKSKSFEQDSRKRTQSWTALRVGAILAATSSVAYLNWHNGQIDNEPKLD

SEQ ID NO: 47 - protein, 65 aa, Artificial Sequence
  Polypeptide
MSKTRLNTIRAYGSTVPKSKSFEQDSRKRTQSWTALRVGAILAATSSVAYLNWHNGQIDNEPKLD

SEQ ID NO: 48 - protein, 54 aa, Artificial Sequence
  Polypeptide
MGSTVPKSKSFEQDSRKRTQSWTALRVGAILAATSSVAYLNWHNGQIDNEPKLD

SEQ ID NO: 49 - protein, 45 aa, Artificial Sequence
  Polypeptide
MFEQDSRKRTQSWTALRVGAILAATSSVAYLNWHNGQIDNEPKLD

SEQ ID NO: 50 - protein, 35 aa, Artificial Sequence
  Polypeptide
MSWTALRVGAILAATSSVAYLNWHNGQIDNEPKLD

SEQ ID NO: 51 - protein, 25 aa, Artificial Sequence
  Polypeptide
MLAATSSVAYLNWHNGQIDNEPKLD

SEQ ID NO: 52 - protein, 86 aa, Artificial Sequence
  Polypeptide
MRSAARVINKSCSGSALSRRCLRKSSLSMSMRYLSTSNIGVRKGFNGQGKSSNKTMLFLAAGASAVAGIGLLSQFSDSLQNATKEE

SEQ ID NO: 53 - protein, 66 aa, Artificial Sequence
  Polypeptide
MLRKSSLSMSMRYLSTSNIGVRKGFNGQGKSSNKTMLFLAAGASAVAGIGLLSQFSDSLQNATKEE

SEQ ID NO: 54 - protein, 54 aa, Artificial Sequence
  Polypeptide
MLSTSNIGVRKGFNGQGKSSNKTMLFLAAGASAVAGIGLLSQFSDSLQNATKEE

SEQ ID NO: 55 - protein, 46 aa, Artificial Sequence
  Polypeptide
MRKGFNGQGKSSNKTMLFLAAGASAVAGIGLLSQFSDSLQNATKEE

SEQ ID NO: 56 - protein, 36 aa, Artificial Sequence
  Polypeptide
MSNKTMLFLAAGASAVAGIGLLSQFSDSLQNATKEE

SEQ ID NO: 57 - protein, 26 aa, Artificial Sequence
  Polypeptide
MGASAVAGIGLLSQFSDSLQNATKEE

SEQ ID NO: 58 - protein, 73 aa, Artificial Sequence
  Polypeptide
MFKSQLRTATARSSFRSLASKLNPQRFNSSKTPLLNATRGSNRSKNSLIALAISLSAVSSSYYLYQKDKFISA

SEQ ID NO: 59 - protein, 53 aa, Artificial Sequence
  Polypeptide
MLNPQRFNSSKTPLLNATRGSNRSKNSLIALAISLSAVSSSYYLYQKDKFISA

SEQ ID NO: 60 - protein, 47 aa, Artificial Sequence
  Polypeptide
MNSSKTPLLNATRGSNRSKNSLIALAISLSAVSSSYYLYQKDKFISA

SEQ ID NO: 61 - protein, 38 aa, Artificial Sequence
  Polypeptide
MATRGSNRSKNSLIALAISLSAVSSSYYLYQKDKFISA

SEQ ID NO: 62 - protein, 28 aa, Artificial Sequence
  Polypeptide
MSLIALAISLSAVSSSYYLYQKDKFISA

SEQ ID NO: 63 - protein, 18 aa, Artificial Sequence
  Polypeptide
MAVSSSYYLYQKDKFISA

SEQ ID NO: 64 - protein, 105 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDC

SEQ ID NO: 65 - protein, 105 aa, Kluyveromyces marxianus
MRSAARVINKSCSGSALSRRCLRKSSLSMSMRYLSTSNIGVRKGFNGQGKSSNKTMLFLAAGASAVAGIGLLSQFSDSLQNATKEELNKPKVSPLEVAKHSSPDD

SEQ ID NO: 66 - protein, 105 aa, Wickerhamomyces anomalus
MFKSQLRTATARSSFRSLASKLNPQRFNSSKTPLLNATRGSNRSKNSLIALAISLSAVSSSYYLYQKDKFISADVPHWKDIELTPEIVSQHNKKDDLWVVLNGQV

SEQ ID NO: 67 - protein, 105 aa, Candida glabrata
MQRYIFKTPHSRVLRSLPRHAINSTTKRGIVSSRSSLNNQFFNSERQRKSYKWFWSIGLTVGLTAAIADNLHNKKDIYNDAKFDSSKPKISPSEVIKHNTPEDCW

SEQ ID NO: 68 - protein, 60 aa, Lachancea thermotolerans CBS 6340
KRLSSHVALFTALAGLTYILGPGREISLIKNDAAKHSKRRHVSATEVIRHNKEDDCWVVI

SEQ ID NO: 69 - protein, 78 aa, Lachancea thermotolerans CBS 6340
MMRLQELRNQRYRDLNLRIRITQSEISTSRTVVTTALPVLMFRLISQKTHVSLSRLFLRGQWRAFSKGAGFRGTQRVK

SEQ ID NO: 70 - protein, 65 aa, Saccharomycodes ludwigii
NNNSRFSLFAAVAATVITVGTTAIITSNKSDASFLSNTSSVLNKPPIDPKEVVKHNKPDDCWIVI

SEQ ID NO: 71 - protein, 60 aa, Saccharomycodes ludwigii
MYKRLISKKVYLNTFNSKANFTGKYLSAPKCSFNCIRGYSKFNGNYNVHSAKKYDNNNNN

SEQ ID NO: 72 - protein, 63 aa, Naumovozyma castellii CBS 4309
KRKYEQTFLWGIPFATVLTLLINQYHTTNIQNDVKLELSSTKSPISTDEVTKHNSENDCWIVI

SEQ ID NO: 73 - protein, 51 aa, Naumovozyma castellii CBS 4309
MLLFIKTSKLKLPKRLLSNFRKTCISPSRNYSRKQTQSFRTVEEYYKAFYK

SEQ ID NO: 74 - protein, 64 aa, Zygosaccharomyces bailii CLIB 213
RRTAAVATAVVAAAAAAAAAGSAAGMSLWGSPIANGTTKDMSKPAISADDVAKHNTDKSIWVVI

SEQ ID NO: 75 - protein, 49 aa, Zygosaccharomyces bailii CLIB 213
MSAMCGRLKPLAQASLRASKRAFNTKSPLRGVQLRPSRCAQRQAHVPFR

SEQ ID NO: 76 - protein, 64 aa, Zygosaccharomyces parabailii
RRTAAVATAVVAAAAAAAAAGSAAGMSLWGSPIANGTTKDMSKPAISADEVAKHNTDKSIWVVI

SEQ ID NO: 77 - protein, 49 aa, Zygosaccharomyces parabailii; identical to SEQ ID NO: 75

SEQ ID NO: 78 - protein, 56 aa, Lachancea mirantina
RILAGAIALGTTAFSINYWADASKIKNNELRDSAKAPVSPFEVVKHDKENDCWVVI

SEQ ID NO: 79 - protein, 43 aa, Lachancea mirantina
MLFRSQSRLMRLSTRLSLKFERQSVRNFGSSAYARSIGGQRRM

SEQ ID NO: 80 - protein, 59 aa, Tetrapisispora phaffii CBS 4417
KLPGNYFYYGILGLFLISASNFACDRYYRNDSMLLKNSKVITPEEVARHNKRDDCWVVI

SEQ ID NO: 81 - protein, 33 aa, Tetrapisispora phaffii CBS 4417
MYNKRFVLNYARFRTFPHRSYSRQYTVNNTKVK

SEQ ID NO: 82 - protein, 62 aa, Saccharomyces eubayanus
KRTQSWTTLSVGTILAAAGSMAYLNWSNDQIGNDPKLDMNKQKISPAEVAKHNSADDCWVVI

SEQ ID NO: 83 - protein, 48 aa, Saccharomyces eubayanus
MLKIKPLLRISRVPEAATLRTSRTRLNTARSYSSAVGKSKSFQQHSGK

SEQ ID NO: 84 - protein, 62 aa, Saccharomyces cerevisiae x Saccharomyces kudriavzevii VIN7
KRTQSWTALSIGAILATAGSMVCLNWYNGQIDNEPKLDMNKQKISPAEVAKHNKADDCWVVI

SEQ ID NO: 85 - protein, 48 aa, Saccharomyces cerevisiae x Saccharomyces kudriavzevii VIN7
MFKYKPLLRMSRSSEAAMLRTSKTRLNTVRSYRSAVTKSKSFEQSSRK

SEQ ID NO: 86 - protein, 62 aa, Saccharomyces cerevisiae
KRTQSWTALRVGAILAATSSVAYLNWHNGQIDNEPKLDMNKQKISPAEVAKHNKPDDCWVVI

SEQ ID NO: 87 - protein, 48 aa, Saccharomyces cerevisiae
MLKYKPLLKISKNCEAAILRASKTRLNTIRAYGSTVPKSKSFEQDSRK

SEQ ID NO: 88 - protein, 62 aa, Saccharomyces paradoxus
KRTQSWTALSVGTILAAASSMAYLNWCNGQIDNEPKLDMNKQKISPAEVAKHNKPDDCWVVI

SEQ ID NO: 89 - protein, 48 aa, Saccharomyces paradoxus
MLKYKPLLKISRNCGAAILRSSKIRLNTIRAYGSVVSESKSFEQGSRK

SEQ ID NO: 90 - protein, 67 aa, Vanderwaltozyma polyspora DSM 70294
GKNYKTTTIVASSALSTTLLAYLYTNNNINNISNDVSIEQLSKKSLKIITPQEVIKHNSKDDCWVVI

SEQ ID NO: 91 - protein, 43 aa, Vanderwaltozyma polyspora DSM 70294
MIRNNITALKVVRQPLKFKNLSRCISTSQISLKNKGYKNYQYG

SEQ ID NO: 92 - protein, 62 aa, Candida glabrata
QRKSYKWFWSIGLTVGLTAAIADNLHNKKDIYNDAKFDSSKPKISPSEVIKHNTPEDCWVVI

SEQ ID NO: 93 - protein, 47 aa, Candida glabrata
MQRYIFKTPHSRVLRSLPRHAINSTTKRGIVSSRSSLNNQFFNSERQ

SEQ ID NO: 94 - protein, 59 aa, Kluyveromyces lactis
GKKVTTGGAFLVAGSVGIALISHFNDSVENGAKVDMTKPKVSPTEVAKHSSPKDCWVVI

SEQ ID NO: 95 - protein, 50 aa, Kluyveromyces lactis
MRSAVRVFGKNRVSGLRFASTSSKSPIHKSSATPRFGRSAKSDYSEFSFG

SEQ ID NO: 96 - protein, 60 aa, Lachancea dasiensis CBS 10888
TRPSGNIALLTSFSVITYALCSNREKSFIKNDLVRDLKRSSICASEVLLHNKEDDCWVVI

SEQ ID NO: 97 - protein, 36 aa, Lachancea dasiensis CBS 10888
MFRQSVLHPAFKRAFDRGRWRALSHSAKQVGARHVT

SEQ ID NO: 98 - protein, 59 aa, Wickerhamomyces ciferrii
YSQNSLIALGISITAIASIYSYNQSQNYLSADVPKWSDIELTPEIVSQHNKKDDCWVVI

SEQ ID NO: 99 - protein, 49 aa, Wickerhamomyces ciferrii
MLRPQLIKSILGSSRLSSKGLSHSSLRLFAGSNKRLLSTLKKTTKSKTY

SEQ ID NO: 100 - protein, 61 aa, Kluyveromyces dobzhanskii CBS 2104
SSRKSTTYGGAFLVASAVAIAFSSQITQSLENGVKVDMGKPSVSPAEVVKHSSPDDCWVVI

SEQ ID NO: 101 - protein, 51 aa, Kluyveromyces dobzhanskii CBS 2104
MRSAVRVFRKNRFPTGLRLSSTASSARQGIALSVGKYNKYNKNGTNGSWKS

SEQ ID NO: 102 - protein, 60 aa, Kluyveromyces marxianus DMKU3-1042
SSNKTMLFLAAGASAVAGIGLLSQFSDSLQNATKEELNKPKVSPLEVAKHSSPDDCWVVI

SEQ ID NO: 103 - protein, 51 aa, Kluyveromyces marxianus DMKU3-1042
MRSAARVINKSCSGSALSRRCLRKSSLSMSMRYLSTSNIGVRKGFNGQGKS

SEQ ID NO: 104 - protein, 65 aa, Kazachstania naganishii CBS 8797
STRRNGTRLVTGAAAVATVLLVSQLYQIENESAQQQPLLPGAPTKSISPSEVAKHNTPDDCWVVI

SEQ ID NO: 105 - protein, 46 aa, Kazachstania naganishii CBS 8797
MLSSRIPGVASKRLLQLARQQQGNAVVGAAVRSRLRASSYSTQSRS

SEQ ID NO: 106 - protein, 59 aa, Zygosaccharomyces mellis
SKNRRMAIALLIAAITAASTVEPFGGHLDNGTKVDMSKPAINPEEVAKHNSPGDCWVVI

SEQ ID NO: 107 - protein, 52 aa, Zygosaccharomyces mellis
MHLLDMLFRWKPVWQLSKVTVRSLRADAGFNGKRSLSGKTRDNKVWFHFPMS

SEQ ID NO: 108 - protein, 64 aa, Kazachstania saulgeensis
KNLRNSLLLITSASALSYYAWNQLNNQSSSDYISNDNGQPSKKKPISPDEVAKHNKPDDCWVVI

SEQ ID NO: 109 - protein, 44 aa, Kazachstania saulgeensis
MQALGRRSIAFRACKQFKFTKISKRALSTSNFTKSRNYYNNNSK

SEQ ID NO: 110 - protein, 58 aa, Naumovozyma castellii CBS 4309
NKAQLMAIAGAGLIATTTLLYSNFNSEIQNESKLAPKRVIDPSEVARHNTPADCWIVI

SEQ ID NO: 111 - protein, 50 aa, Naumovozyma castellii CBS 4309
MLGVSRQCEVTGRKLFSGKVLSKNFKYLSNRKYSLATTKNGAKYESKKFN

SEQ ID NO: 112 - protein, 58 aa, Candida boidinii
NKFNTFLSIITILGIGLTISSSSIISNDSSSSKKNAKKLISVEEFVKHNKPDDCWVVI

SEQ ID NO: 113 - protein, 53 aa, Candida boidinii
MLKQSQKILIKSFNYSNKRIINNSKIGFKNQLFRNYTTNSIKNSINNNNNNAN

SEQ ID NO: 114 - protein, 62 aa, Lachancea fermentati
AKDQKFVYPLVVGALGLGAVAASALHWGSVIDNGPVKDPTKKPIKPEEVSRHCTPEDCWVVI

SEQ ID NO: 115 - protein, 42 aa, Lachancea fermentati
MLGGIRSKFVRNLVTNGARASHRPQGRLLSGSKRYLATHRVA

SEQ ID NO: 116 - protein, 59 aa, Zygosaccharomyces rouxii
SRNRNWATALLVTAVAAASTVKLSAGPLDNGPKLDMSKPAISPDEVAKHNSPEDCWVVI

SEQ ID NO: 117 - protein, 45 aa, Zygosaccharomyces rouxii
MLFRSKPIWQLSRTTARSLRAGINSKRSLAGNARSNNVLSRFSVS

SEQ ID NO: 118 - protein, 56 aa, Wickerhamomyces anomalus
NSLIALAISLSAVSSSYYLYQKDKFISADVPHWKDIELTPEIVSQHNKKDDLWVVL

SEQ ID NO: 119 - protein, 45 aa, Wickerhamomyces anomalus
MFKSQLRTATARSSFRSLASKLNPQRFNSSKTPLLNATRGSNRSK

SEQ ID NO: 120 - protein, 62 aa, Cyberlindnera fabianii
KSAHNNKLLPALGFLGASASIAALLLNNTEPFIRLDAPTQGPLYTPDDVSAHNTKDDLWVVL

SEQ ID NO: 121 - protein, 40 aa, Cyberlindnera fabianii
MFHSTSLRVSRAIRASKVIHSSPLRGPRSKVRQLTTASIK

SEQ ID NO: 122 - protein, 62 aa, Kazachstania africana CBS 2517
PRFQTRNLLFYTTLTAASLSSFYLYSDIDGPCTISNEAITTRLILPEEVAKHKTPDDCWVVI

SEQ ID NO: 123 - protein, 40 aa, Kazachstania africana CBS 2517
MFNSRYVFQLSRLTLRKRPFDGISVRRMSTRHFVSNSKIP

SEQ ID NO: 124 - protein, 29 aa, 1FCB/1-511
EPKLDMNKQKISPAEVAKHNKPDDCWVVI

SEQ ID NO: 125 - protein, 60 aa, Cyberlindnera jadinii
MSIRNATIIGFGAGAITSAYYLSQLQQQSPILLESPSVIGELTPEVVSKHNKRDDLWIVL

SEQ ID NO: 126 - protein, 31 aa, Cyberlindnera jadinii
MFRSAAQRLRSVGKNGRLTRLVHTTRAQAKM

SEQ ID NO: 127 - protein, 60 aa, Lachancea quebecensis
RSPFSYTAIFTVFATLTYTMGQYRDATFIKNDAAFDLKRQGVSASEVIRHNRKDDCWVVI

SEQ ID NO: 128 - protein, 57 aa, Kuraishia capsulata CBS 1993
ATSALAIGLSSAIVLSVTSSIISNDIKYEDKLFPEPKLIPVDEFLKHNKPDDCWVVL

SEQ ID NO: 129 - protein, 36 aa, Kuraishia capsulata CBS 1993
MFRSNFIRNSRSAFKQFSKRSYSWRSVSTSPRLAKH

SEQ ID NO: 130 - protein, 59 aa, Torulaspora delbrueckii
PKNRHFITGAVALASLSTLLWISDRPTIDNGTVKDSTKKSIDPKEVEKHNAPDDCWVVI

SEQ ID NO: 131 - protein, 38 aa, Torulaspora delbrueckii
MFARSTRTVAQVVRKAPSRSLLARRGISGLHIQRNGLP

SEQ ID NO: 132 - protein, 60 aa, Komagataella pastoris
QSSRKILIKGGLVIGIAVAATTATIWSQSLASPISNDDERKVTPEELAKHNSGTDCWVAI

SEQ ID NO: 133 - protein, 46 aa, Komagataella pastoris
MSGRLMLQGYRSSIKATVFAARNLRPLQASRKLSSILPKVGSEKSQ

SEQ ID NO: 134 - protein, 60 aa, Lachancea nothofagi CBS 11611
VGRLGFVAGIAIFSAGLYTFEKRKFASVIKNDVRQDSKKPGILPSEVARHDKDNDCWVVI

SEQ ID NO: 135 - protein, 39 aa, Lachancea nothofagi CBS 11611
MFRIRLQRGCLLRTNQTIVRRQLRPFTTNIRTVNAQRVV

SEQ ID NO: 136 - protein, 68 aa, Naumovozyma dairenensis CBS 421
RSYHTYLYMSLTGISFYSLAIIILNNSIHDRHIDNDSKMELDPVKSKTPITPNELMKHNTPEDCWVVI

SEQ ID NO: 137 - protein, 56 aa, Naumovozyma dairenensis CBS 421
MRRRLITSLVKKGNFQSKRSLHLTNNNLYISSSILKKHPNTGNIPGIQSIENAYKR

SEQ ID NO: 138 - protein, 151 aa, Wickerhamomyces anomalus
MFKSQLRTATARSSFRSLASKLNPQRFNSSKTPLLNATRGSNRSKNSLIALAISLSAVSSSYYLYQKDKFISADVPHWKDIELTPEIVSQHNKKDDLWVVLNGQVYDLTDFLPNHPGGQKIIIRYAGKDATKIFVPIHPPDTIEKFIPPEK

SEQ ID NO: 139 - protein, 151 aa, Cyberlindnera_fabianii
MFHSTSLRVSRAIRASKVIHSSPLRGPRSKVRQLTTASIKSAHNNKLLPALGFLGASASIAALLLNNTEPFIRLDAPTQGPLYTPDDVSAHNTKDDLWVVLNGKVYDLTEFLPNHPGGQRIILKYAGKDASKIFNPIHPPDAIDKYLPPEK

SEQ ID NO: 140 - protein, 157 aa, Naumovozyma_castellii
MLGVSRQCEVTGRKLFSGKVLSKNFKYLSNRKYSLATTKNGAKYESKKFNKAQLMAIAGAGLIATTTLLYSNFNSEIQNESKLAPKRVIDPSEVARHNTPADCWIVINGVVYDLTSFIPVHPGGADIIKSNAGKDVTAIFEPIHAPGVIEKYLPPKC

SEQ ID NO: 141 - protein, 174 aa, Saccharomycodes_ludwigii
MYKRLISKKVYLNTFNSKANFTGKYLSAPKCSFNCIRGYSKFNGNYNVHSAKKYDNNNNNNNSRFSLFAAVAATVITVGTTAIITSNKSDASFLSNTSSVLNKPPIDPKEVVKHNKPDDCWIVINNVVYDLTDFIAKHPGGPDIIQSNAGKDVSAIFNPLHASDVIDKYIKPEC

SEQ ID NO: 142 - protein, 158 aa, Candida glabrata
MQRYIFKTPHSRVLRSLPRHAINSTTKRGIVSSRSSLNNQFFNSERQRKSYKWFWSIGLTVGLTAAIADNLHNKKDIYNDAKFDSSKPKISPSEVIKHNTPEDCWVVIDGYVYDLTNFIALHPGGPDIIKTNAGKDVTAIFDPIHPPDAIEKYIKPEQ

SEQ ID NO: 143 - protein, 159 aa, Saccharomyces cerevisiae
MLKYKPLLKISKNCEAAILRASKTRLNTIRAYGSTVPKSKSFEQDSRKRTQSWTALRVGAILAATSSVAYLNWHNGQIDNEPKLDMNKQKISPAEVAKHNKPDDCWVVINGYVYDLTRFLPNHPGGQDVIKFNAGKDVTAIFEPLHAPNVIDKYIAPEK

SEQ ID NO: 144 - protein, 160 aa, Kluyveromyces marxianus
MRSAARVINKSCSGSALSRRCLRKSSLSMSMRYLSTSNIGVRKGFNGQGKSSNKTMLFLAAGASAVAGIGLLSQFSDSLQNATKEELNKPKVSPLEVAKHSSPDDCWVVIDGFVYNLTEFISAHPGGPAIIENNAGKDVTAIFGPIHAPDVIEKYIAPEN

SEQ ID NO: 145 - protein, 506 aa, Artificial Sequence
  LDH
MNKQKISPAEVAKHNKPDDCWVVINGYVYDLTRFLPNHPGGQDVIKFNAGKDVTAIFEPLHAPNVIDKYIAPEKKLGPLQGSMPPELVCPPYAPGETKEDIARKEQLKSLLPPLDNIINLYDFEYLASQTLTKQAWAYYSSGANDEVTHRENHNAYHRIFFKPKILVDVRKVDISTDMLGSHVDVPFYVSATALCKLGNPLEGEKDVARGCGQGVTKVPQMISTLASCSPEEIIEAAPSDKQIQWYQLYVNSDRKITDDLVKNVEKLGVKALFVTVDAPSLGQREKDMKLKFSNTKAGPKAMKKTNVEESQGASRALSKFIDPSLTWKDIEELKKKTKLPIVIKGVQRTEDVIKAAEIGVSGVVLSNHGGRQLDFSRAPIEVLAETMPILEQRNLKDKLEVFVDGGVRRGTDVLKALCLGAKGVGLGRPFLYANSCYGRNGVEKAIEILRDEIEMSMRLLGVTSIAELKPDLLDLSTLKARTVGVPNDVLYNEVYEGPTLTEFEDA

SEQ ID NO: 146 - protein, 502 aa, Artificial Sequence
  LDH
MATGSDSPRSISVDEFVKHNSQNDCWIAINGKVYDFTDFIPNHPGGVPPLVNHAGYDGTKLYEKLHPKGTIEKFLPKDKFLGVLDGEAPKLEADYLVDDDEQERLDYLNNLPPLSSIQNVYDFEYLAKKILPKDAWAYYSCGADDEITMRENHYAYQRVYFRPRICVDVKEVDTSYEMLGTKTSVPFYVSATALAKLGHPDGECSIARGAGKEGVVQMISTLSSMSLDEIAAARIPGATQWFQLYINEDRNVAKGLVKHAEDLGMKAIFITVDAPSLGNREKDKRLKFVNDTDVDLGDSADRNSGASKALSSFIDASVSWNDVKAVKSWTKLPVLVKGVQTVEDVIEAYDAGCQGVVLSNHGGRQLDTAPPPIELLAETVPTLKRLGKLRPDFEILIDGGVKRGTDILKAVAIGGQDVRVSVGMGRPFLYANSCYGEAGVRKLIQNLKDELEMDMRLLGVTKMDQLSSKHVDTKRLIGRDAINYLYDNVYSPIETVKFNNED

SEQ ID NO: 147 - protein, 505 aa, Artificial Sequence
  LDH
MVTNDVTPKSKNIISVEDFVKHNSKDDCWIAINGKVFDVTEFIPDHPGGEIPLISHAGYDGTKVYEKIHPKGTIEKFLPEDKFLGILDGDAPKLEADYLIDDDEEERLKYIENIPPISNIQNSYDFEYIAKHILPKDAWAYYSCGSDDEISMRENHYAYQRVYFRPRICVNVKEVDTSTTLLGTPTSVPFYVSATALAKLGHPDGECSIARGAGKEGVIQMISTLSSNSLEEIAQARIPGATQWFQLYINENRETAKRMVKEAEDLGMKAIFITVDAPSLGNREKDKREKFVDDTDVDLGDGVDRNSGASKALSAFIDCSVDWNDVKEVKSWTKLPVLIKGVQTVEDVIEAYDAGCQGVVLSNHGGRQLDTAPPPIELLAETVPVMKKLNKLRPDFEILLDGGVKRGTDILKAVAIGGKDIRVGVGLGRPFLYANSCYGEDGVRKLIQILKDELEMDMRLLGVTKISDLTPKFVDTRRLIGREAVNYLYDDVYNHIPTVKFRNDD

SEQ ID NO: 148 - protein, 503 aa, Artificial Sequence
  LDH
MQLSSESPALPKRIVPVAEFVKHNRPDDCWVAIRGQVYDMTEFLPHHPGGQSPIIRYSGHDATELFEQLHPKGTIEKHLPKEKHLGQLDGPAPTLEVAEDEFEEERLENVANMPNVNEVMNLHDFEYIAKKILPKGAWAYYSSGADDEVSMRENHYAYQRIYFRPRVLVDVSKVDTSTTLLGTPTSVPFYVSATALAKLGHPDGECSIARGAGKEGVIQMISTLASNSLEEIAAARVPGATQWFQLYVNEDRNVAFEMVKKAEKLGIKAIFVTVDAPSLGNREKDARVKFEGESDVQKSNEVVRSQGASRALSSFIDTRLTWDDVKKIKQSTKLPVLIKGVQRLEDVVQAVDDGFDGVVLSNHGGRQLDTAPPPVELLAEVVPELKRRNKLRPDFEIFIDGGVRRGTDILKALALGGQNVRVGVGLGRPFLYANSSYGENGVRKAIQLLKDELEMDMRLLGVRNLRELDETFVDTRRLIGRDAPDELYNQLYSPLKTVKFRNE

SEQ ID NO: 149 - protein, 499 aa, Artificial Sequence
  LDH
MAPLTGVEIAKHNKPDDCWVIVHGRAYDVTEFLPEHPGGTKIILKYAGKDATEEFDPIHPPDTLEKYLPKSKHLGPVDMSTVVQEKAEESPEEKERMKRIQEMPALEQCYNLLDFEAVARRVMKKTAWGYYSSAADDEITLRENHSAFHRIWFRPRILVDVEKVDFSTTMLGTPCSVPFYITATALGKLGHVEGEVVLTRAAHKHNVIQMIPTLASCAFDEIVDAAGPGQVQWLQLYVNKDRAITQRIVQHAEKRGCKGLFITVDAPQLGRREKDMRMKFTEQGSNVQSGQATDTSQGAARAISSFIDPALSWADIPWFRSITKMPIVLKGVQRVEDVVRAAEAGVQGVVLSNHGGRQLEFARSGIEILAETMPVLRKLGLDNKIEVYIDGGIRRATDILKALCLGAKGVGIGRPFLYAMSAYGQEGVERAMQLLKDEMEMGMRLIGAQTIADLNPSMVDARSLFNHSSPPLDSLSHAVYEPLVNPSQRLSVTGEKAKL

SEQ ID NO: 150 - protein, 503 aa, Artificial Sequence
  LDH
MQLSSESPALPKRIVPVDEFVKHNRPDDCWVAIRGQVYDMTEFLPQHPGGQSPIIRYSGHDATELFEQLHPKGTIEKNLPKDKHLGQLDGPAPTLEVAEDEFEEERLENVANMPNVNEVMNLHDFEYIAKKILPKGAWAYYSSGADDEVSMRENHYAYQRIYFRPRVLVDVSKVDTSTTLLGTPTSVPFYVSATALAKLGHPDGECSIARGAGKEGVIQMISTLASNSLEEIAAARVPGATQWFQLYVNEDRNVAFEMVKKAERLGIKAIFVTVDAPSLGNREKDARVKFEGESDVQKSNEVVRSQGASRALSSFIDTRLTWDDVIKIKQSTKLPVLIKGVQRLEDVVRAVDDGFDGVVLSNHGGRQLDTAPPPVELLAEVVPELRRRNKLRPDFEIFIDGGVRRGTDILKALALGGQNVRVGVGLGRPFLYANSAYGENGVRKAIQLLKDELEMDMRLLGVRNLRELDETFVDTRRLIGRDAPDELYNQLYSPLKTVKFRNE

SEQ ID NO: 151 - protein, 506 aa, Artificial Sequence
  LDH
MLLNEVEISTNNSISIEEFKKHNKKDDCWVAINGKVYDVTEFIPNHPGGQSPLIKHGGYDATKVYENLHPKGTIEKFLPKDKFKGILIDEAPKLPSDYAVDDFEAQRLDNIENKPPLSSIQNTYDFEKIAKNILPREAWAYYSCGSDDEISMRENHYAYQRVYFKPRICINVKEVDTSTNILGSKSSVPFYVTATALAKLGHPDGECSIAKGAGKEGVIQMISTLSSMSLKEIADSRIPGATQWFQLYINEDRNITKNLVKEAENLGMKAIFVTVDAPSLGNREKDKRLKFVDDTDVDLGGDVDRTSGASKALSSFIDASLDWSDIRLIKQWTKLPVLIKGVQTVDDVIEAFDIGCQGVVLSNHGGRQLDTAPPPIELLAETVPIMKKLGKLRPDFEILVDGGVKRGTDILKAVAIGGNDIKVSVGLGRPFLYANSAYGEAGVRKLIQILKDELEMDMRLLGVTKIDQLTSKYVDTRRLIGREASNYLYDNVYTPIDTVKFKNEDD

SEQ ID NO: 152 - protein, 502 aa, Artificial Sequence
  LDH
MAQLTGVEVAKHNKSDDCWVIVHGRAYDVTEFLPEHPGGTKIILKYAGKDATEEFDPIHPPDTLEKYLPKSKHLGPVDMSTVVKEKHEESPEEQERMKRIQEMPLLEQCYNLLDFEAVARRVMKKTAWGYYSSAADDEITLRENHSAFHRIWFRPRILIDVEKVDFSTTMLGTPCSIPFYVTATALGKLGHVEGEVVLTRSAHKHNVVQMIPTLASCSFDDIVDAAAPDQVQWLQLYVNKDRAITQRIVQHAEKRGCKGLFITVDAPQLGRREKDMRMKFTDEGSNVQNGQATDNSQGAARAISSFIDPSLSWADIPWFRSITKMPIVLKGVQRVEDVVKAAEAGVQGVVLSNHGGRQLEFARSAIEVLAETMPVLRELGLENKIEIYVDGGVRRATDILKALCLGAKGVGIGRPFLYAMSAYGQDGVDRAMQLLKDEMEMGMRLIGARTIAELNPGMVDARSLFNHASPPSDSLAHAVYDPLVNPSQRLAVTGEKPEKAKL

SEQ ID NO: 153 - protein, 499 aa, Artificial Sequence
  LDH
MAPLTGVEVAKHNKPNDCWVIVHGRAYDVTEFLPEHPGGTKIILKYAGKDATEEFDPIHPPDTLEKYLPKSKHLGPVDMTTVVKEKVEESPEERERLERIKNMPLLEQCYNLLDFEAVARRVMKKTAWGYYSSAADDEITLRENHSAFHRIWFRPRILVDVEKVDFSTTMLGTPVSIPFYVTATALGKLGHVEGEVVLTRAAAKHNVIQMIPTLASCAFDEIVDAASGGQVQWLQLYVNKDRAITQRIVQHAEKRGCKGLFITVDAPQLGRREKDMRMKFTDQGSNVQSGQATDTSQGAARAISSFIDPALSWEDIPWFRSITKMPIVLKGVQRVEDVIKAAEAGVQGVVLSNHGGRQLEFARSGIEILAETMPVLRKLGLENKIEVYIDGGIRRATDILKALCLGARGVGIGRPFLYAMSTYGLDGVDRAMQLLKDEMEMGMRLIGARNIAELNASMVDARSLFNHAAPPIDTLSSAVYDPLLNPPQRSPVKGDKAKL

SEQ ID NO: 154 - protein, 506 aa, Artificial Sequence; identical to SEQ ID NO: 151
  LDH

SEQ ID NO: 155 - protein, 502 aa, Artificial Sequence; identical to SEQ ID NO: 152
  LDH

SEQ ID NO: 156 - protein, 499 aa, Artificial Sequence; identical to SEQ ID NO: 153
  LDH

SEQ ID NO: 157 - protein, 5 aa, Artificial Sequence
  N-Terminus
DAAKH

SEQ ID NO: 158 - protein, 5 aa, Artificial Sequence
  N-Terminus
TSSVL

SEQ ID NO: 159 - protein, 5 aa, Artificial Sequence
  N-Terminus
DVKLE

SEQ ID NO: 160 - protein, 5 aa, Artificial Sequence
  N-Terminus
GTTKD

SEQ ID NO: 161 - protein, 5 aa, Artificial Sequence
  N-Terminus
NELRD

SEQ ID NO: 162 - protein, 5 aa, Artificial Sequence
  N-Terminus
DSMLL

SEQ ID NO: 163 - protein, 5 aa, Artificial Sequence
  N-Terminus
DPKLD

SEQ ID NO: 164 - protein, 5 aa, Artificial Sequence
  EPKLD
EPKLD

SEQ ID NO: 165 - protein, 5 aa, Artificial Sequence
  N-Terminus
DVSIE

SEQ ID NO: 166 - protein, 5 aa, Artificial Sequence
  N-Terminus
DAKFD

SEQ ID NO: 167 - protein, 5 aa, Artificial Sequence
  N-Terminus
GAKVD

SEQ ID NO: 168 - protein, 5 aa, Artificial Sequence
  N-Terminus
DLVRD

SEQ ID NO: 169 - protein, 5 aa, Artificial Sequence
  N-Terminus
DVPKW

SEQ ID NO: 170 - protein, 5 aa, Artificial Sequence
  N-Terminus
GVKVD

SEQ ID NO: 171 - protein, 5 aa, Artificial Sequence
  N-Terminus
ATKEE

SEQ ID NO: 172 - protein, 5 aa, Artificial Sequence
  N-Terminus
ESAQQ

SEQ ID NO: 173 - protein, 5 aa, Artificial Sequence
  N-Terminus
GTKVD

SEQ ID NO: 174 - protein, 5 aa, Artificial Sequence
  N-Terminus
DNGQP

SEQ ID NO: 175 - protein, 5 aa, Artificial Sequence
  N-Terminus
ESKLA

SEQ ID NO: 176 - protein, 5 aa, Artificial Sequence
  N-Terminus
DSSSS

SEQ ID NO: 177 - protein, 5 aa, Artificial Sequence
  N-Terminus
GPVKD

SEQ ID NO: 178 - protein, 5 aa, Artificial Sequence
  N-Terminus
GPKLD

SEQ ID NO: 179 - protein, 5 aa, Artificial Sequence
  N-Terminus
DVPHW

SEQ ID NO: 180 - protein, 5 aa, Artificial Sequence
  N-Terminus
DAPTQ

SEQ ID NO: 181 - protein, 5 aa, Artificial Sequence
  N-Terminus
EAITT

SEQ ID NO: 182 - protein, 5 aa, Artificial Sequence
  N-Terminus
ESPSV

SEQ ID NO: 183 - protein, 5 aa, Artificial Sequence
  N-Terminus
DAAFD

SEQ ID NO: 184 - protein, 5 aa, Artificial Sequence
  N-Terminus
DIKYE

SEQ ID NO: 185 - protein, 5 aa, Artificial Sequence
  N-Terminus
GTVKD

SEQ ID NO: 186 - protein, 5 aa, Artificial Sequence
  N-Terminus
DDERK

SEQ ID NO: 187 - protein, 5 aa, Artificial Sequence
  N-Terminus
DVRQD

SEQ ID NO: 188 - protein, 5 aa, Artificial Sequence
  N-Terminus
DSKME

SEQ ID NO: 189 - protein, 18 aa, Artificial Sequence
  N-Terminus
MHHHHHHHHQNATKEELN